(19) United States
(12) Patent Application Publication
(10) Pub. No.: US 2025/0382589 A1
(43) Pub. Date: Dec. 18, 2025

(54) SYNTHETIC OPERONS FOR THE PRODUCTION OF 2-MERCAPTOETHANE SULFONATE (COENZYME M) AND METHODS OF USING THE SAME

(71) Applicant: Board of Regents of the University of Nebraska, Lincoln, NE (US)

(72) Inventors: Nicole Roswitha Buan Murphy, Lincoln, NE (US); Connor John Hines, Lincoln, NE (US); Rebecca Roston, Lincoln, NE (US); Dylan Dewayne Erickson, Lincoln, NE (US)

(21) Appl. No.: 19/236,605

(22) Filed: Jun. 12, 2025

Related U.S. Application Data
(60) Provisional application No. 63/659,271, filed on Jun. 12, 2024.
(60) Provisional application No. 63/714,225, filed on Oct. 31, 2024.

(51) Int. Cl. C12N 9/00; C12N 9/04; C12N 9/10; C12N 9/16; C12N 9/88; C12N 15/82; C12P 11/00

(52) U.S. Cl. CPC C12N 9/00; C12N 9/0006; C12N 9/1025; C12N 9/1085; C12N 9/1096; C12N 9/16; C12N 9/88; C12N 15/8261; C12P 11/00; C12Y 101/01095; C12Y 101/01338; C12Y 203/03015; C12Y 205/01076; C12Y 206/01001; C12Y 206/01052; C12Y 206/01077; C12Y 301/03071; C12Y 401/01079; C12Y 404/01019

(57) ABSTRACT

Disclosed herein are polynucleotides comprising sequences encoding coenzyme M synthase (ComF) linked to a heterologous regulatory element and methods of using the same. The polynucleotides may comprise synthetic operons comprising additional sequences encoding enzymes, e.g., a taurine-pyruvate aminotransferase, a sulfoacetaldehyde acetyl transferase, or a sulfopyruvate decarboxylase. Also disclosed herein are recombinant prokaryotic cells, e.g., recombinant bacterial, e.g., E. coli, or archaeal cells, e.g., Methanosarcina acetivorans with improved tolerance to oxidative stress.

### CROSS-REFERENCE TO RELATED APPLICATIONS

The present application claims priority to U.S. Provisional Patent Application No. 63/659,271 that was filed Jun. 12, 2024, and U.S. Provisional Patent Application No. 63/714,225, filed Oct. 31, 2024, the entire contents of each which are hereby incorporated by reference.

### STATEMENT REGARDING FEDERALLY SPONSORED RESEARCH

This invention was made with government support under IOS-1449525 and IOS-1938948 awarded by the National Science Foundation and NEB-30-133, NEB-30-138 awarded by the United States Department of Agriculture, and RR-17675 awarded by the National Center for Research Resources. The government has certain rights in the invention.

### REFERENCE TO A SEQUENCE LISTING

A Sequence Listing accompanies this application and is submitted as an xml file of the sequence listing named “165275_00038.xml” which is 211,848 bytes in size and was created on Jun. 12, 2025. The sequence listing is electronically submitted via Patent Center and is incorporated by reference herein in its entirety.

### BACKGROUND OF THE INVENTION

Coenzyme M, also known as 2-mercaptoethanesulfonate (MESNA), is an antioxidant that can be chemically synthesized, but is sensitive to oxidation in the environment, which limits the use of chemically synthesized CoM. Biosynthesis of antioxidants, e.g., CoM, has the advantage of deploying the antioxidants in vivo at the site of oxidative damage in recombinant organisms. In addition, biosynthesis of CoM could increase the efficiency of CoM production, compared to standard chemical synthesis. However, complete biosynthesis of CoM has not been demonstrated and there is a need in the art for compositions and methods for the biosynthesis of CoM.

### BRIEF SUMMARY OF THE INVENTION

A polynucleotide comprising a sequence encoding a coenzyme M synthase (ComF) operably linked to at least one heterologous regulatory element.

In an aspect of the current disclosure, cells comprising a polynucleotide comprising a sequence encoding a coenzyme M synthase (ComF) operably linked to at least one heterologous regulatory element are provided.

In an aspect of the current disclosure, methods are provided. In some embodiments, the methods comprise introducing a polynucleotide disclosed herein into a cell.

In an aspect of the current disclosure, methods of increasing aerial tissue growth in a plant are provided. In some embodiments, the methods comprise expressing a polynucleotide of this disclosure in cells of the plant and growing the plant.

In an aspect of the current disclosure, methods of increasing dry weight of a plant are provided. In some embodiments, the methods comprise expressing a polynucleotide of this disclosure in cells of the plant and growing the plant.

In an aspect of the current disclosure, methods of producing coenzyme M (CoM) are provided. In some embodiments, the methods comprise culturing cells comprising a polynucleotide of this disclosure and harvesting the CoM from the cultured cells.

In an aspect of the current disclosure, recombinant archaeal cells are provided. In some embodiments the recombinant archaeal cells comprise a polynucleotide encoding a cysteate synthase or a sequence encoding a sulfopyruvate decarboxylase, wherein the sequence encoding a cysteate synthase or the sulfopyruvate decarboxylase is operably linked to a heterologous regulatory element.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A, 1B, 1C, and 1D show the Roles of coenzyme M and Hdr in methanogenesis. Panel a, structure of Coenzyme M, 2-mercatoethanesulfonate. Panel b, Coenzyme M is essential in methanogenesis, where it serves as a C1-carrier and half of the terminal electron acceptor CoM-S—S-CoB heterodisulfide. Coenzyme M is in bold orange text. Methylotrophic methanogenesis pathway is in cyan. Acetoclastic methanogenesis is in magenta. Reactions common to all methanogenesis pathways are in black arrows. Heterodisulfide reductase, Hdr (shaded oval) reduces the terminal electron acceptor CoM-S—S-CoB using two electrons to regenerate CoM-SH and CoB-SH thiols. Panel c, Effect of AhdrABC deletion on methyltrophic growth. Green indicates upregulated genes and increased metabolic flux; red indicates decreased mRNA transcripts and metabolic flux; gray arrows indicate unchanged mRNA transcript abundance and metabolic flux. Panel d, energy conservation reactions in M. acetivorans. CH₃CO-CoA, acetyl coenzyme A; CH₃—H₄MPT, methyl tetrahydromethanopterin; CH₃—S-CoM, methyl coenzyme M; CH₃—X, methylotrophic substrates such as methanol, methyl sulfides, methylamines, methoxy compounds; CoB-SH, coenzyme B thiol; CoM-SH, coenzyme M thiol; e−, electrons; Fd, ferredoxin; Fd(red), reduced ferredoxin; F₄₂₀, deazaflavin cofactor F₄₂₀; F₄₂₀(red), reduced F₄₂₀; MPh, methanophenazine; MPh(red), reduced methanophenazine; ΔμH⁺, reaction coupled to a transmembrane proton gradient; Δ82 Na⁺, reaction coupled to a transmembrane sodium gradient.

FIG. 2 shows CoM biosynthesis pathways in methanogens. In Methanosarcinales and Class II methanogens (gray box), CoM synthesis begins with O-phospho-L-serine. Cysteate synthase (MA3297) catalyzes a β-elimination of phosphate from O-phospho-L-serine followed by a β-addition of sulfite to produce L-cysteate. Aspartate aminotransferase (MA1816) catalyzes a transamination reaction between L-cysteate and α-ketoglutarate to form sulfopyruvate and L-glutamate. Sulfopyruvate decarboxylase (ComDE, MA3298) enzymatically decarboxylates sulfopyruvate to form sulfoacetaldehyde. Enzymatic conversion from sulfoacetaldehyde to CoM is currently undocumented although it is believed that a reductive reaction between sulfide and sulfoacetaldehyde could form CoM autocatalytically. Class I methanogens instead synthesize CoM from phosphoenol pyruvate using ComABC enzymes which are lacking in Class II methanogens.

FIGS. 3A, 3B, 3C, 3D, 3E, and 3F show plasmids and strain validation. Panel a, the putative com genetic locus in M. acetivorans (red). Oligonucleotide primers used for cloning and strain validation are shown by gray arrows. Panel b, plasmid map for pNB710 which results in constitutive or tetracycline-inducible overexpression of comDE (MA3298). Panel c, plasmid map for pNB714 which results in high constitutive overexpression of comDE. Panel d, plasmid map for pNB711, which increases the com locus copy number when introduced into host strains. Panel e, validation of DhdrABC deletion by PCR screen. Panel f, validation of plasmid pNB711 integration by PCR screen. All primers are listed in Table 1. +, positive control; −, negative control; kb, kilobase; M, DNA marker.

FIGS. 4A, 4B, 4C, 4D, 4E, and 4F show the effect of com overexpression on growth of M. acetivorans on methanol and methanol+acetate. Panels a-c show methylotrophic growth curves on 125 mM methanol. Panels d-f show mixotrophic growth curves on 125 mM methanol plus 40 mM acetate. Panels a and d, comparison of parent versus AhdrABC strains overexpressing PₜₑₜcomDE⁺. Panels b and e, parent versus AhdrABC strains overexpressing P_{mcr}comDE⁺. Panels c and f, parent versus AhdrABC strains overexpressing com⁺. Parent and AhdrABC curves are the same in each panel for comparison. Error bars have been omitted for clarity. Each data point represents the average of at least four biological replicates. OD, optical density at 600 nm.

FIGS. 5A, 5B, 5C, 5D, 5E, 5F, and 5G show deletion of hdrABC and overproduction of CoM results in increased resistance to oxidative stress. Panel a, growth of strains in medium with methanol as sole energy source and sulfide omitted. Error bars are removed for clarity. Data for parent (blue) and zhdrABC (orange) strains in panel a are graphed from Salvi et al. as part of the same experiment for comparison.¹⁶ Subsequent panels show growth of strains in the same medium. Panel b, Growth of each strain when culture headspace contained 5% O₂. Panels c-g, cultures were grown to OD=0.4 under unstressed conditions as in Panel a, then fresh H₂O₂ to the indicated concentration was added at time 0 h. Each data point represents the average of at least four biological replicates. Error bars indicate standard deviation. OD, optical density at 600 nm.

FIGS. 6A, 6B, and 6C show overexpression of com genes results in changes to thiol pools in MeOH-grown cells. Panel a, quantification of reduced CoM-SH and oxidized CoM (Panel b) and from parent (n=12), AhdrABC (n=9), com⁺ (n=10), and AhdrABC com⁺ (n=8) strains. Panel c, ratio of CoM-SH vs total CoM. Error bars indicate standard deviation. P values indicated were calculated by two-tailed T test versus parent strain.

FIG. 7 shows overexpression of com genes results no change to coenzyme B pools in MeOH-grown cells. Quantification of free CoB-SH and total CoB from parent (n=7) and com⁺ (n=5) strains. CoB could not be accurately quantified from AhdrABC and AhdrABC com⁺ strains.

FIGS. 8A, 8B, 8C, 8D, 8E, 8F, and 8G show exemplary synthetic operons of the instant disclosure.

FIG. 9 shows exemplary proteins encoded by the disclosed synthetic operons and the source of the sequences.

FIG. 10 shows exemplary biosynthetic pathways representing the disclosed synthetic operons.

FIG. 11 shows that although CoM (ArA) possesses a redox potential similar to Glutathione (GSH), increases of biomass yield are observed in Arabidopsis thaliana being supplemented with CoM in comparison to GSH and a Control containing no additional antioxidant.

FIG. 12 shows the effects of CoM on Non-Photochemical Quenching, the process by which plants protect themselves from negative effects of high light conditions is shown to decrease with CoM supplemented in sterile media indicating less need for protection.

FIG. 13 shows plasmid map of pCH003 which was constructed using NE Builder HiFi Assembly to assemble two genes (sNB25 and sNB34) on a pET24a backbone. These genes are hypothesized to establish a metabolic pathway for the production of the CoM using a high flux metabolite present in E. coli (pyruvate) and taurine (“supplemental compound (SC)”.

FIG. 14 shows Western blot comparing four samples of BL21-derivative E. coli expressing pET24a as a Vector-Only Control (VOC) and pCH003 at two concentrations of IPTG, 0 mM and 0.5 mM. Lysate was separated using SDS-PAGE and identified using a direct action 6×-His Tag Monoclonal Antibody. Both genes inserted of pCH003 are being produced in this system. FIG. 14B shows Protein expression confirmed additionally through a Proteomics Analysis using LC-MS. The lysate of pCH003 expressing BL21-derivative E. coli induced using 0 mM and 0.5 mM IPTG was separated using SDS-PAGE. The gel was fixed using Coomassie stain containing Methanol.

FIG. 15 shows protein expression confirmed additionally through a Proteomics Analysis using LC-MS. The lysate of pCH003 expressing BL21-derivative E. coli induced using 0 mM and 0.5 mM IPTG was separated using SDS-PAGE. The gel was fixed using Coomassie stain containing Methanol, bands of gel were cut out and submitted for proteomics analysis.

FIG. 16 shows VOC and pCH003 expressing E. coli K-12 AxxxX (ΔtauD) were grown in Defined Media, induced at OD600 of 0.4-0.6, grown for an hour, then aliquoted and given SC for the final hour of their growth to promote production of ArA. Cultures were then normalized to OD600 of 1.0 and 1:9 innoculated into fresh media. This culture was used to 1:9 innoculate media in a 96 well plate and treated with Cumene Hydroperoxide and incubated in a Tecan Plate Reader for 16 hours. Under oxidative stress, the lag time of VOC cells increases in the presence of the SC while the lag time of pCH003 expressing cells shortens in the prescence of SC indicating improved fitness towards the oxidative stress.

FIG. 17 shows Another use for the OD600=1.0 generated through the procedure described in FIG. 6 was an acute stress assay. Here we take 40 μL of the aforementioned culture and spread on a warm LB Plate and allow to dry. Four sterile absorption disks are placed at the vertices of a square on the plate. One disk is given 5 μL H2O while the other three are given 5 μL 2.45 mM H₂O₂ to create zones of clearing. After 16 hours incubation the plates are photographed and their zones of clearing are measured. Here we can see the beneficial effects of pCH003 when the K-12 E. coli are incubated with SC.

FIG. 18 shows pathways for CoM biosynthesis. Class I methanogens begin with phosphoenolpyruvate, while members of the orders Methanosarcinales and Methanomicrobiales derive CoM from O-phospho-L-serine.(27) The com^{syn} pathway utilizes a ubiquitous high-flux metabolite, pyruvate, and a supplement able compound, taurine, via taurine-pyruvate aminotransferase (Tpa) from B. wadsworthia and MA3299 from M. acetivorans (ComF) to synthesize CoM.

FIGS. 19A, 19B, 19C, and 19D show preincubation of E. coli with CoM protects cells from ROS. Panel a, growth curves of wild-type E. coli K12 showing recovery from oxidative stress with cumene hydroperoxide. Panel b, population lag times calculated from Panel a. Panel c, growth curves of E. coli K-12 ΔtauD showing recovery from ROS stress. Panel d, population lag times calculated from Panel c. No significance (NS) was marked for p>0.24 and no growth (NG) was marked for cultures which did not grow. Error bars represent standard deviation; they were omitted for clarity in panels a and c. Five biological replicates were used for all assays (n=5).

FIGS. 20A and 20B show genomic loci used to create the com^{syn} operon. Panel a, sNB25 is derived from the genomic sequence of MA3299 (cyan) from M. acetivorans. The region surrounding MA3299 includes both MA3297 and MA3298 that establish the metabolic pathway from O-phospho-L-serine to sulfoacetaldehyde. MA3300 is predicted to be a ThiS/MoaD-like protein involved with sulfur transfer, which may assist MA3299 as a sulfur donor, however it is separated from MA3299 by 1890 bp and unlikely to be co-transcribed. The function of the large, unannotated area between MA3299 and MA3300 is unknown. Panel b, sNB34 is derived from the genomic sequence of tpa (dark blue) from B. wadsworthia. While the surrounding genes do not seem to exist in an operon with tpa, the enzyme alanine dehydrogenase (Ald) establishes a cyclic metabolic loop with Tpa by converting alanine back to pyruvate while reducing NAD⁺ to NADH.

FIGS. 21A and 21B show mechanistic insights from computational modeling of MA3299. Panel a, MA3299 structure predicted using Alphafold with two 4Fe-4S cluster cofactors and substrate sulfoacetaldehyde (SAA) fit using Autodock. Panel b, the electrostatic map of MA3299 shows an electropositive pocket (blue) which leads to the docked SAA substrate.

FIGS. 22A, 22B, 22C and 22D show IPTG-induced dual expression of sNB34 (tpa) and sNB25 (comF) from pCH003. Panel A, map of plasmid pCH003 encoding the com^{syn} operon expressing sNB34 (tpa) and sNB25 (comF). Panel B, Western blot of cell extracts from E. coli expressing pCH003 (NB521) grown in defined medium induced using 0 mM and 0.5 mM IPTG. A dark band corresponding to Tpa appears slightly above the 50 kDa mark while a lighter band corresponding to ComF appears slightly below the 50 kDa mark. The two bands appearing at 30 kDa and 15 kDa are hypothesized to be degradation products of Tpa. BL21/pCH003 was grown in defined medium and induced with IPTG. Panel C shows % protein coverage and Panel D shows % total spectra of Tpa and ComF as detected by LC-MS/MS.

FIGS. 23A, 23B, and 23C show enzymatic synthesis of CoM in E. coli lysate. Panel a, RP-HPLC separation of thiols synthesized by E. coli cell lysates expressing com^{syn} (red) and when spiked with CoM standard (dark blue). Standards for cysteine (5.2 min, gray), and CoM (5.5 min, cyan) are plotted for reference. Panel b, enzymatic synthesis of CoM by E. coli com^{syn} lysate over time. Panel c, quantification of CoM after 5 h from lysis buffer, E. coli com^{syn} lysate, lysate spiked with CoM standard, soluble E. coli com^{syn} extract, and insoluble E. coli com^{syn} fraction. Error bars in panels b & c represent uncertainty estimated from a 4-point calibration curve with R²=0.99998.

FIGS. 24A, 24B, and 24C show E. coli K-12 ΔtauD acute oxidative stress assay. Panel a, representative agar plate illustrating the phenotype of the VOC strain grown in the presence of 0.1 mM taurine when stressed with H₂O₂. Panel b, representative agar plate illustrating the phenotype of the pCH003 (com^{syn}-expressing) strain grown in the presence of 0.1 mM taurine when stressed with H₂O₂. Panel c, quantification of the acute stress phenotypes shown in panels a and b showing taurine and com^{syn}-dependent protection from ROS. Error bars represent standard deviation (n=5).

FIGS. 25A, 25B, 25C, and 25D show E. coli K-12ΔtauD chronic oxidative stress assay. Panel a, growth curve in the absence of taurine supplementation. Panel b, growth curve when taurine is supplied in culture medium. Panel c, lag times, defined as the time it took to achieve OD600=0.1, show taurine and com^{syn}-dependent resistance to ROS. Panel d, population doubling times indicate com^{syn} strains grow slightly slower than the VOC strains after recovery from ROS, but growth is unaffected under unstressed conditions in the absence and presence of taurine. Cumene or CuHO₂ (140 μM) were added as indicated. No significance (NS) was marked for p>0.24. Error bars represent standard deviation; they were omitted for clarity in panels a and b. Five biological replicates were used for all assays (n=5).

FIG. 26 shows the interaction between CoM and E. coli. Within our study, CoM was either exogenously provided to E. coli through the media or synthesized endogenously through com^{syn}. We were able to show that exogenous CoM enters the cell, presumably through an ABC transporter or permease involved with trafficking sulfonate containing molecules (TauABC, SsuABC) or small thiol containing molecules (TcyJLN, TcyP, CyuP). CoM can be utilized as a source of sulfur, likely through either sulfonate catabolism (SsuDE, TauD), thiol catabolism (SseA, CsdA, SufS, IscS), or both. CoM was also shown to benefit E. coli when exposed to sources of oxidative stress either through mitigation of the ROS directly or by detoxifying alkene-containing reactive aldehydes generated through lipid peroxidation. While we did supply the oxidative species, H₂O₂ or CuHO₂, in order to test the resilience of com^{syn} E. coli, there are endogenous sources of ROS which CoM could mitigate as well. We expect CoM to form homodisulfides and heterodisulfides when acting as an antioxidant and generate adducts with the alkene groups of select lipid peroxidation products.

FIGS. 27A, 27B, 27C, 27D, 27E, and 27F show predicted monomer and dimer models of MA3299 (ComF). Structures generated using Alphafold2, Autodock4, and ColabFold then displayed using Pymol. Panel a, a model of MA3299 generated using AlphaFold2 with structures of SAA and 4Fe-4S clusters inserted using Autodock. Panel b, electrostatic surface of the same model from panel a, SAA, circled using a dashed yellow line, can be observed inside the enzyme. Panel c, dimeric model of MA3299 generated using ColabFold using an alignment of the monomer model of MA3299 to place ligands in analogous locations. Panel d, electrostatic surface of one of the two MA3299 chains of the dimer model shows how the dimer model obscures the entrance to the active site. Panel e, conserved residues (>95%) of the dimer model around the obscured entrance to the active site displayed as space filling to show how this particular area is conserved on both portions of the protein interacting with one another. Panel f, electrostatic surface of the dimer model of MA3299 shows that the large entrance to the active site is entirely obscured from the front in a space filling model.

FIG. 28 shows neighbor-joining phylogenetic tree of COG1900d sequences. Sequences were retrieved from NCBI using BLAST. The sequences were aligned using the MUSCLE algorithm, and the tree was constructed in MEGA11 using the Poisson model with uniform rates among sites and pairwise deletion of gaps. The numbers at each branch represent percentages of bootstrapping support after 1000 replications. Brackets show taxonomic grouping by order with the red branches representing proteins with Cys200 and black branches representing proteins that instead have Ser200 when aligned with MA3299 from M. acetivorans.

FIG. 29 shows MUSCLE Alignment of COG1900d sequences. The amino acid sequences used in the phylogenetic tree of FIG. S2 aligned using MUSCLE and displayed in Snapgene with residues conserved at a rate of over 95% highlighted in yellow. The bar of colors above the sequences represents a gradient of non-conserved (dark blue) to conserved (dark red). Above the bar of colors are annotations of the features mentioned in the text including dimerization residues, proposed catalytic residues, t-stacked phenylalanine residues (red asterisks), the ferredoxin arm, and Ser404 marked with a black asterisk. Amino acid sequences shown, from top to bottom, are SEQ ID NOs: 1 and 78-100.

FIGS. 30A, 30B, 30C, 30D, 30E, and 30F show predicted solvent accessibility of the MA3299 active site. Panel a, when viewing the inside of the monomeric construct of MA3299, SAA can be observed sitting within the active site. Panel b, the same simulation from panel a zoomed out with the entrance to the active site circled using a dashed yellow line. Panel c, SAA (docked to the monomeric construct) inside of the dimeric construct of MA3299 viewed from the same angle as panel a. Panel d, the same simulation from panel c zoomed out with the entrance to the active site circled using a dashed yellow line. Panel e, the entrance to the active site of the dimer model circled using a dashed yellow line. Panel f, amino acid side chains present in >95% of sequences shown as space filling, an arrow points to the sulfur atom of Cys95 and SAA is circled using a dashed yellow line

FIGS. 31A, 31B, 31C, 31D, 31E, and 31F show predicted active site geometry of MA3299 with docked SAA and 4Fe-4S clusters. Panel a, polar contacts between the docked SAA molecule and the protein. Panel b, a display of the trio of conserved cysteine and serine residues which surround the aldehyde group of SAA with distances shown. Panel c, a different angle of the distance between Cys200 and Cys202 shows SAA resting between the two residues. Panel d, an angled view from Cys200 to the nearest Fe—S cluster showing two aromatic phenylalanine residues situated on either side. Panel e, edge-to-face T-shaped π-π interaction between Phe263 and Phe365. Panel f, electron tunnelling distances between the nearest Fe—S cluster and Cys200, Cys202, and Ser404.

FIGS. 32A, 32B, 32C, and 32D show that CoM can serve as an S source for E. coli. E. coli K-12 and a ΔtauD mutant strain was streaked for isolation on M9 (—S) agar plates with different sulfur sources. Panel a, H₂O negative control. Growth in Panel a indicates S carryover after washing cells three times with phosphate-buffered saline. Panel b, 100 μM cysteine (Cys). Panel c, 100 μM taurine (Tau). Panel d, 100 μM coenzyme M (CoM).

FIGS. 33A and 33B show CoM can serve as an S source for E. coli. Panel a, E. coli K-12 wild-type and panel b, ΔtauD mutant strains were grown in M9 minimal media then inoculated into sulfur-free M9 minimal media with either no sulfur source (+H2O), CoM (+1× CoM), or Tau (+2×Tau). Twice as much taurine as CoM was provided in order to stoichiometrically balance the sulfur content of each source. Both strains can utilize CoM as a source of sulfur for growth. (n=6) FIGS. 34A, 34B, 34C, and 34D show regressions of the lag times reported in FIG. 19. Lag times of wild-type (a, b) and ΔtauD (c, d) E. coli put through an exponential (a, c) or logarithmic (b, d) regression analysis as is appropriate when analyzing the exponential growth of microorganisms. Using the exponential regression line in panel a, if 65 μM CuHO₂ was applied to a wild-type culture incubated with CoM (+CoM) the lag time is predicted to be 6.313 h. When this lag time is applied to the logarithmic regression line in panel b for a wild-type culture incubated without CoM 49.73 μM CuHO₂ is calculated. Using these regression analyses (both R²>0.97) we assert that the presence of CoM in wild-type cultures resulted in the mitigation of ˜15 μM CuHO₂. (n=6, except ΔtauD −CoM+75 μM where one culture did not grow)

FIGS. 35A, 35B, and 35C show E. coli K-12 wild-type & ΔgshA acute oxidative stress assay. Panel a, representative agar plates illustrating the phenotypes of wild-type (left) and ΔgshA (right) strains transformed with VOC when stressed with 100 mM H₂O₂. Panel b, representative agar plates illustrating the phenotype of wild-type (left) and ΔgshA (right) strains transformed with pCH003 (com^{syn}-expressing) when stressed with 100 mM H₂O₂. Panel c, quantification of the zones of clearing shown in panels a and b caused by H₂O₂ show that com^{syn} expression provides increased resistance to ROS when compared to the endogenous antioxidant glutathione (p=005). Error bars represent standard deviation (n=6).

FIGS. 36A, 36B, 36C, 36D, 36E, 36F, and 36G show Plasmids produced containing synthetic operons for coenzyme M biosynthesis. a|pCH008 and Operon 1. b|pCH009 and Operon 2. c|pCH010 and Operon 2.1. d|pCH003 and Operon 3. e|pCH004 and Operon 4. f|pCH005 and Operon 5. g|pCH011 and Operon 6.

FIG. 37 shows Western Blot Analysis of SDS-PAGE Separated E. coli K-12:pCH003 Lysate. E. coli K-12:pCH003 was grown in Defined Media and induced under 0 mM and 0.5 mM IPTG. A Western Blot utilizing 6×His direct action antibodies shows the presence of both MA3299 (48.5 kDa) in the form of a thinner lighter band beneath the wide dark spot and Q9APM5 (50.7 kDa) in the form of a wide, dark spot in the Defined Media culture. The darker spots at 30 and 15 kDa and in 0.5 mM IPTG lane are likely degradation products of Q9APM5 since E. coli K-12 possesses higher concentrations of proteases than a protein overexpression strain such as BL21 E. coli. The band at 50 kDa in the 0 mM IPTG lane is Q9APM5 expressed through leaky expression.

FIG. 38 shows Western Blot Analysis of SDS-PAGE Separated E. coli K-12:pCH003 Lysate. E. coli K-12:pCH003 was grown in Defined Media and induced under 0 mM and 0.5 mM IPTG. A Western Blot utilizing 6×His direct action antibodies shows the presence of both MA3299 (48.5 kDa) in the form of a thinner lighter band beneath the wide dark spot and Q9APM5 (50.7 kDa) in the form of a wide, dark spot in the Defined Media culture. The darker spots at 30 and 15 kDa and in 0.5 mM IPTG lane are likely degradation products of Q9APM5 since E. coli K-12 possesses higher concentrations of proteases than a protein overexpression strain such as BL21 E. coli. The band at 50 kDa in the 0 mM IPTG lane is Q9APM5 expressed through leaky expression.

FIG. 39 shows protein model of MA3299. This structure was predicted using the Deepmind Alphafold program and the two 4Fe-4S Cluster cofactors as well as sulfoacetaldehyde were fit into the structure using Autodock.

FIG. 40 shows Placement of two 4Fe-4S clusters within MA3299. These two 4Fe-4S clusters were predicted to fit within an arm of MA3299 using Autodock. They are respectively coordinated using the eight cysteine residues: Cys333, Cys336, Cys339, Cys374 and Cys345, Cys364, Cys367, Cys370.

FIG. 41 shows Placement of sulfoacetaldehyde within MA3299. The presence of Thr56 and Asn206 could serve to orient sulfoacetaldehyde with hydrogen bonding. a|Sulfoacetaldehyde is predicted to fit into MA3299 near Thr56 and Asn206 using Autodock. b|A representation of what this hydrogen bonding would look like using Chemdraw.

FIG. 42 shows Electrostatic surface map of MA3299. This electrostatic surface map, generated using the protein modelling software Pymol, shows a hydrophilic section of MA3299 which is colored blue due to a prevalence of basic residues.

FIG. 43 shows Placement of two cysteine residues within MA3299. The presence of Cys200 and Cys202 allow for a sulfur donor to transfer sulfur onto one of these cysteines which then transfers the sulfur onto sulfoacetaldehyde creating coenzyme M.

FIG. 44 shows Western blot showing expression of MA3299 and Q9APM5. A Western Blot utilizing 6×His direct action antibodies shows the presence of both MA3299 (48.5 kDa) in the form of a thinner lighter band beneath the wide dark spot and Q9APM5 (50.7 kDa) in the form of a wide, dark spot in the Defined Media culture which was not given iron and sulfur supplementation. Columns from left to right correspond to: BenchMark™ His-tagged Protein Standard, E. coli BL21:pCH003&pDB1282 soluble fraction, E. coli BL21:pCH003&pDB1282 insoluble fraction.

FIG. 45 shows Structural comparison between MA3299 (gray) and MJ1681 (lavender). Both protein structures were generated using Alphafold and their peptide backbones were superimposed upon each other using Pymol. The placements of the two 4Fe-4S clusters and sulfoacetaldehyde were generated using Autodock on MA3299.

FIG. 46 shows MJ1681 Alphafold construct with ligands localized using Autodock on MA3299. Of the two cysteine residues, Cys72 and Cys166, only Cys166 is located near the catalytic site. Cys72 can be seen on the outside of the enzyme, far from the active site.

FIGS. 47A and 47B show Fluorescence output from HPLC separation of thiol standards. HPLC was used in the registration and measurement of various thiols through mBBr derivatization. Cysteine elutes at 5.1 minutes, coenzyme M elutes at 5.48 minutes, and excess mBBr elutes at 6.35 minutes. a|100 μM cysteine. b|100 μM coenzyme M.

FIG. 48 shows Fluorescence output from HPLC separation of various enzymatic assay samples. Through HPLC separation of thiols the concentration a 148% increase of coenzyme M at 5.5 minutes can be observed in the cell lysate when compared to the cell free lysis buffer only sample. Supplementation of 400 μM coenzyme M increases the size of the peak. a|The chromatogram from an HPLC separation of mBBr-derivatized thiols in a sample of crude lysate 5 hours after the start of the enzymatic assay. b| Integrations of the 5.5 min peak from samples 5 hours after the start of the enzymatic assay. The integrations were translated to coenzyme M concentration using a standard curve of various coenzyme M concentrations.

FIG. 49 shows ΔtauD E. coli K-12 Growth Curve. pCH003 expresses both sNB24 and sNB25 through Operon 3, which is shown here to improve the recovery time of E. coli K-12 in the presence of taurine after experiencing chronic oxidative stress from being incubated with 140 μM cumene hydroperoxide. The improvement in recovery time demonstrates this production and antioxidant protection of Coenzyme M. Error bars represent standard deviation with 5 biological replicates.

FIG. 50 shows E. coli K-12 Acute Stress Assay. pCH003 expression on it's own shows no decrease in the Zone of Clearing in comparison with the Empty Vector. When these strains are grown in the presence of 0.1 mM taurine the Empty Vector strain experiences an increase in the average zone of clearing size while the operon 3 expressing strain experiences a decrease in the average zone of clearing size. This indicates that the expression of Q9APM5 and MA3299 in the presence of taurine allows the cells to grow in conditions of increased oxidative stress. Error bars represent standard deviation with 5 biological replicates.

### DETAILED DESCRIPTION OF THE INVENTION

Oxidative stress is ubiquitous for all organisms whether they grow aerobically or anaerobically. The inventors hypothesized that Coenzyme M (CoM), a low-molecular weight thiol used as a methyl carrier by anaerobic methane-producing archaea (methanogens), could also be used as an antioxidant to promote growth of aerobic organisms. However, the metabolic pathways for the synthesis of CoM have not been completely resolved in methanogens, and the critical last step has remained elusive for the past two decades. The inventors discovered the identity of the final enzyme in the CoM biosynthetic pathway in Methanosarcina acetivorans, which is referred to as “ComF.” The inventors further discovered that ComF expressed with taurine-pyruvate aminotransferase (Tpa) in aerobically grown Escherichia coli, converts sulfoacetaldehyde into CoM. Thus, polynucleotides or operons for producing CoM in aerobic bacteria or other cells, such as plant cells, and methods of using the polynucleotides and cells are provided. In addition, the operons and polynucleotides may be used to generate CoM and compositions comprising CoM.

### Polynucleotides

Accordingly, in an aspect of this disclosure, polynucleotides are provided. In some embodiments, the polynucleotides comprise a sequence encoding a coenzyme M synthase (ComF) operably linked to at least one heterologous regulatory element.

The inventors discovered that “MA3299” of the Methanosarcina acetivorans genome encodes a CoM synthase (ComF). The sequence of the ComF synthase may be SEQ ID NO: 1, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 1 (sNB25).

As used herein, “operably linked” refers to a functional linkage between two or more sequences such that activity at or on one sequence affects activity at or on the other sequence(s). For example, an operable linkage between a polynucleotide of interest, e.g., a sequence encoding a ComF synthase of the instant disclosure, and a regulatory element (e.g., a promoter) is a functional link that allows for expression of the polynucleotide of interest.

A “heterologous regulatory element,” as used herein, refers to a regulatory element, e.g., cis-acting regulatory elements or trans-acting regulatory elements, that is “heterologous” to the particular polynucleotide of interest. For example, a promoter or ribosome entry site not found in Methanosarcina acetivorans, e.g., a T7 bacteriophage promoter, is a heterologous regulatory element.

Synthetic operons for the production of CoM are disclosed herein, each of which, requires the generation of sulfoacetaldehyde, which is a substrate used by ComF to generate CoM. Therefore, the disclosed polynucleotides may further comprise a sulfoacetaldehyde-producing enzyme. The sulfoacetaldehyde-producing enzyme may include, but is not limited to, a taurine-pyruvate aminotransferase, a sulfoacetaldehyde acetyl transferase, or a sulfopyruvate decarboxylase.

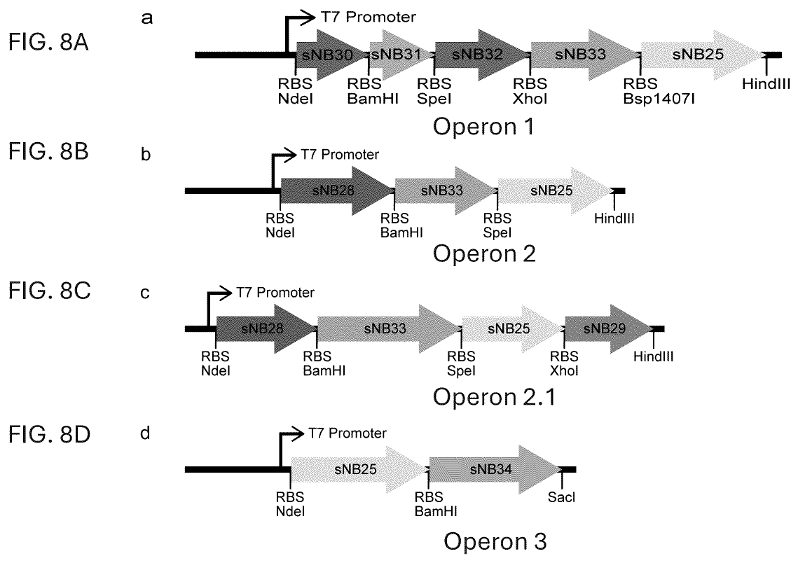

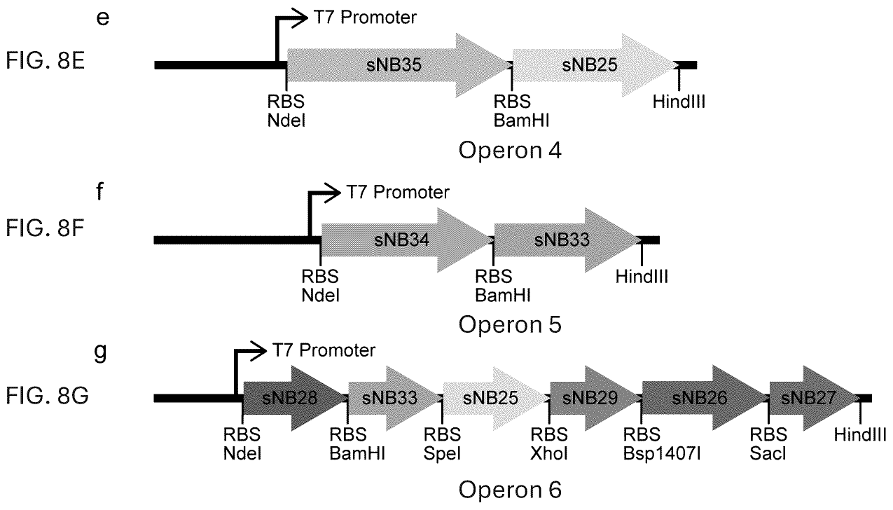

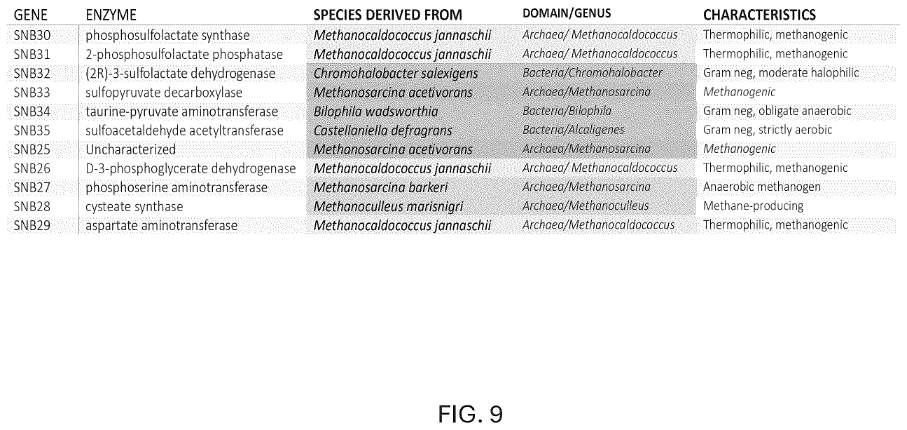

The taurine-pyruvate aminotransferase may comprise SEQ ID NO: 3 (sNB34) or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 3. See FIG. 8D, Operon 3.

The inventors discovered that placing the taurine-pyruvate aminotransferase 5′ to the ComF improved ComF expression. Further, the inventors discovered that operably linking the taurine-pyruvate aminotransferase to a promoter and placing an internal ribosome entry site (IRES) between the taurine-pyruvate aminotransferase and the ComF synthase improved expression of ComF. The promoter may be located 5′ to the taurine-pyruvate aminotransferase.

The sulfoacetaldehyde acetyl transferase may comprise SEQ ID NO: 5 (sNB35) or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 5. See FIG. 8E, Operon 4.

The sulfopyruvate decarboxylase may comprise SEQ ID NO: 6 (sNB33) or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 6. See FIG. 8, Operons 1, 2, 2.1, 5, and 6.

The polynucleotides may further comprise at least one sequence encoding a D-3-phosphoglycerate dehydrogenase (sNB26), a phosphoserine aminotransferase (sNB27), a cysteate synthase (sNB28), an aspartate aminotransferase (sNB29), a phosphosulfolactate synthase (sNB30), a 2-phosphosulfolactate phosphatase (sNB31), a (2R)-3-sulfolactate dehydrogenase (sNB32), or a taurine-pyruvate aminotransferase (sNB34).

The polynucleotides may further comprise sequences encoding a phosphosulfolactate synthase, a 2-phosphosulfolactate phosphatase, and a (2R)-3-sulfolactate dehydrogenase (Operon 1).

The polynucleotides may further comprise a sequence encoding a cysteate synthase (Operon 2).

The polynucleotides may further comprise a sequence encoding an aspartate aminotransferase (Operon 2.1).

The polynucleotides may further comprise sequences encoding a cysteate synthase, an aspartate aminotransferase, a D-3-phosphoglycerate dehydrogenase, and a phosphoserine aminotransferase (Operon 6).

The D-3-phosphoglycerate dehydrogenase may comprise SEQ ID NO: 7, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 7 (sNB26).

The phosphoserine aminotransferase may comprise SEQ ID NO: 8, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 8 (sNB27).

The cysteate synthase may comprise SEQ ID NO: 9, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 9 (sNB28).

The aspartate aminotransferase may comprise SEQ ID NO: 10, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 10 (sNB29).

The phosphosulfolactate synthase may comprise SEQ ID NO: 11, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 11 (sNB30).

The 2-phosphosulfolactate phosphatase may comprise SEQ ID NO: 12, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 12 (sNB31).

The (2R)-3-sulfolactate dehydrogenase may comprise SEQ ID NO: 13, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 13 (sNB32).

The taurine-pyruvate aminotransferase may comprise SEQ ID NO: 3, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 3 (sNB34).

The at least one heterologous regulatory element may be a promoter, e.g., a plant promoter, including, but not limited to, Cauliflower Mosaic Virus 35S (CaMV35S) promoter, Arabidopsis thaliana Act2 promoter, Oryza sativa Act-1 promoter, A. thaliana UBQ1 promoter, Panicum virgatum Ubi1 promoter, P. virgatum Ubi2 promoter, or Zea mays Ubi1 promoter.

The promoter may be a general eukaryotic promoter, e.g., a cytomegalovirus (CMV) promoter, an EF1a promoter, a CAG promoter, a phosphoglycerate kinase (PGK) promoter, tetracycline response element (TRE) promoter, Human U6 nuclear promoter (U6), and upstream activator sequence (UAS) promoter.

The polynucleotides may comprise a binding site for a repressor, e.g., lac repressor. The binding site may comprise a lac operator, e.g., SEQ ID NO: 65, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 65.

The polynucleotides may comprise leading sequences. In one example, the polynucleotides comprise a 5′ leading sequence, e.g., one of SEQ ID NOs: 73-77, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to one of SEQ ID NO: 73-77.

The polynucleotides may comprise or consist of one of SEQ ID NOs: 66-72, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to one of SEQ ID NO: 66-72.

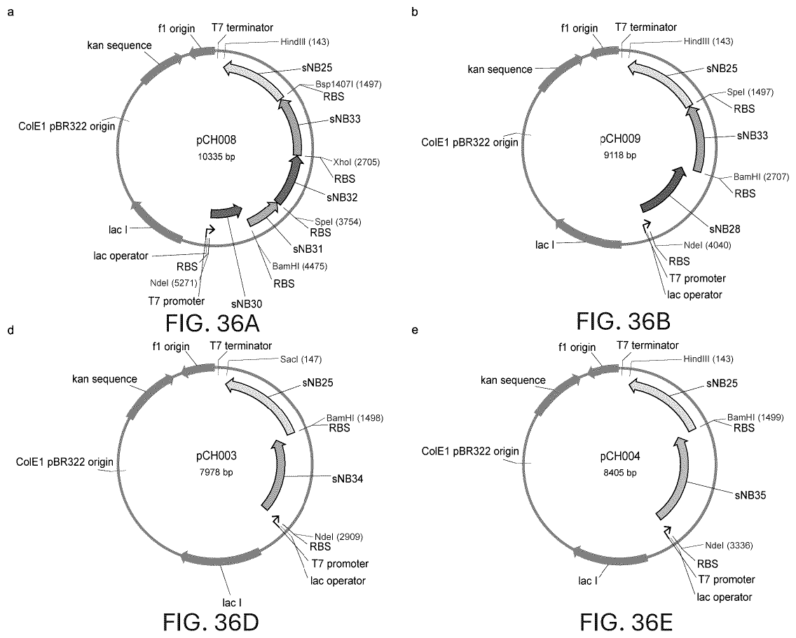

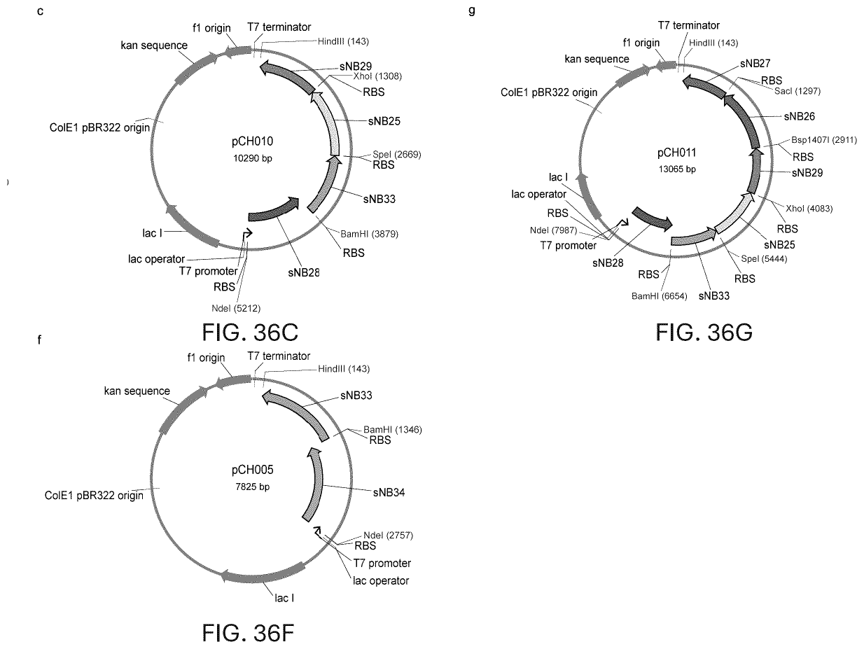

The polynucleotides may comprise ribosome binding sequences, e.g., TAAGGAGGT, before each individual polypeptide encoding sequence, e.g., as shown in FIG. 36. The RBS may comprise TAAGGAGGT or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to TAAGGAGGT.

The polynucleotides may comprise a stop codon at the 3′ end of each polypeptide encoding sequence in the polynucleotide. The stop codon may comprise an ochre stop codon (TAA), an opal stop codon (TGA) or both opal and ochre stop codons (TGATAA). The stop codons may comprise an amber codon (TAG).

### Systems

The disclosed synthetic operons may have some individual elements, e.g., sequences encoding enzymes, separated into different discrete polynucleotides. Accordingly, in an aspect of the current disclosure, systems are provided. The systems comprise at least one polynucleotide comprising (i) a sequence encoding a coenzyme M synthase (ComF); and (ii) a sequence encoding a sulfoacetaldehyde-producing enzyme, each as described above.

Methods of Introducing the Polynucleotides into a Cell

In an aspect of the current disclosure, methods of introducing the disclosed polynucleotides into a cell are provided. As used herein, “introducing” refers to any method to allow the disclosed polynucleotides to enter a cell, e.g., transfection, transduction, or any other suitable modality to allow the polynucleotide access through the cellular membrane to be expressed in the cell or to be incorporated into the genome of the cell.

The cell may be a prokaryotic cell, e.g., a bacterium, or a eukaryotic cell, including, but not limited to, a yeast cell, a plant cell, a vertebrate cell, e.g., a fish or mammal cell.

Disclosed herein are polynucleotides that are optimized for expression in E. coli, and may be suitable for expression in other prokaryotes or simple eukaryotes, e.g., yeast: SEQ ID NOs: 23-36 or sequences with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NOs: 23-36.

The introduced polynucleotides may allow for expression of the polypeptides and production of CoM. Production of CoM may lead to increased resistance of the cell to reactive oxygen species. Increased resistance to reactive oxygen species is relative to a control cell that is not capable of expressing CoM. The increased production of CoM may be due to imparting the ability to make CoM to a cell that normally could not make CoM so an increase in production may be any amount above 0 or may be a 5%, 10%, 15%, 20%, 25% or more increase in production of CoM as compared to a control cell (a similar or parent cell not having the CoM polynucleotides introduced into the cell). The increase in production of CoM may lead to an increase in resistance to reactive oxygen metabolites including a 5%, 10%, 15%, 20%, 35% or more increase in resistance as compared to a control cell in which the polynucleotides were not introduced.

### Methods of Increasing Aerial Tissue Growth in a Plant or Increasing the Dry Weight of a Plant

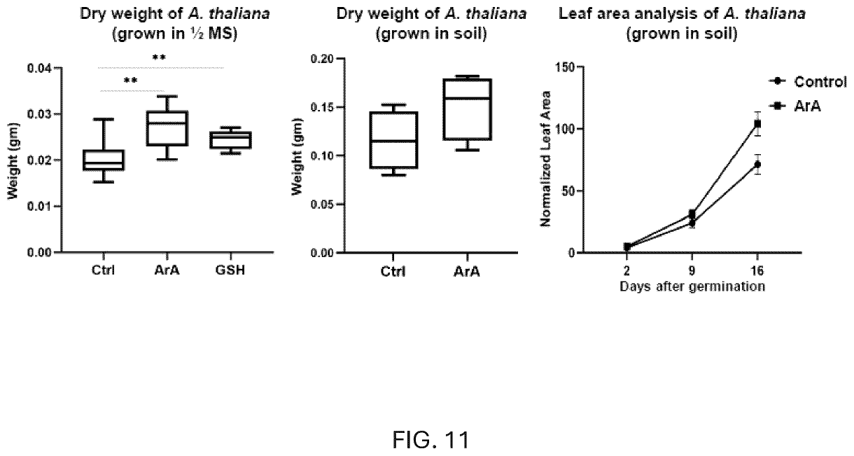

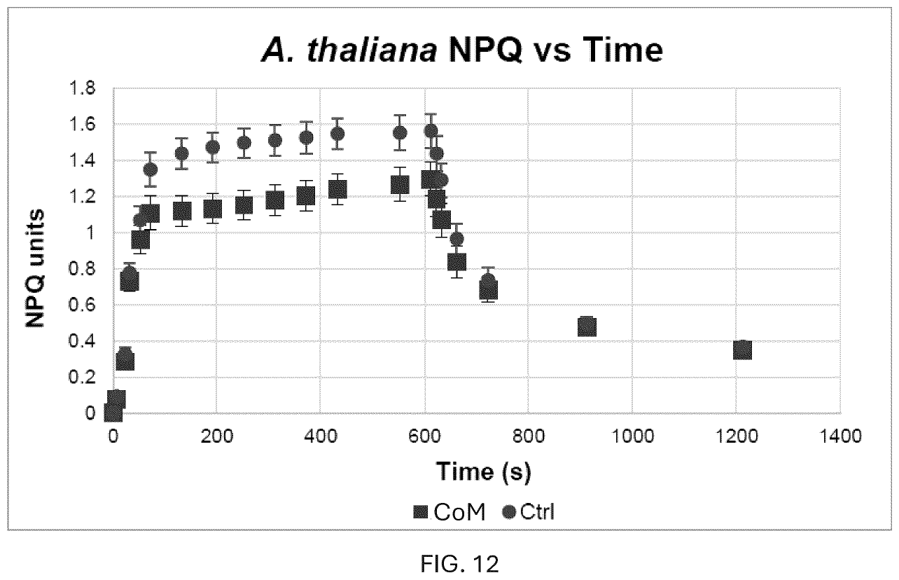

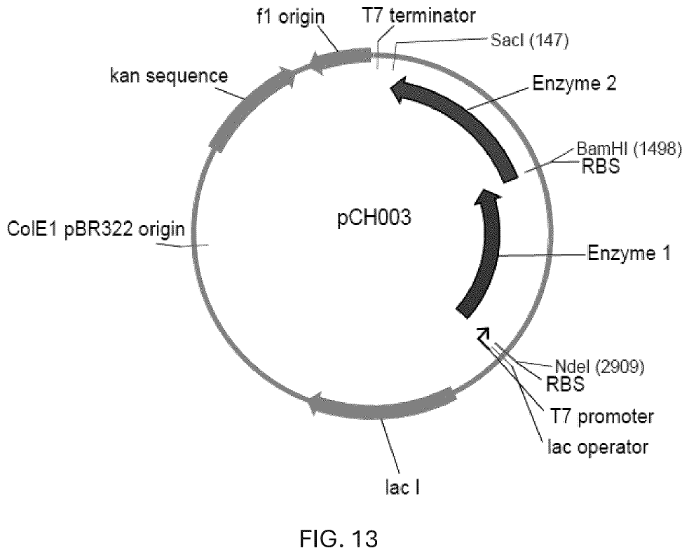

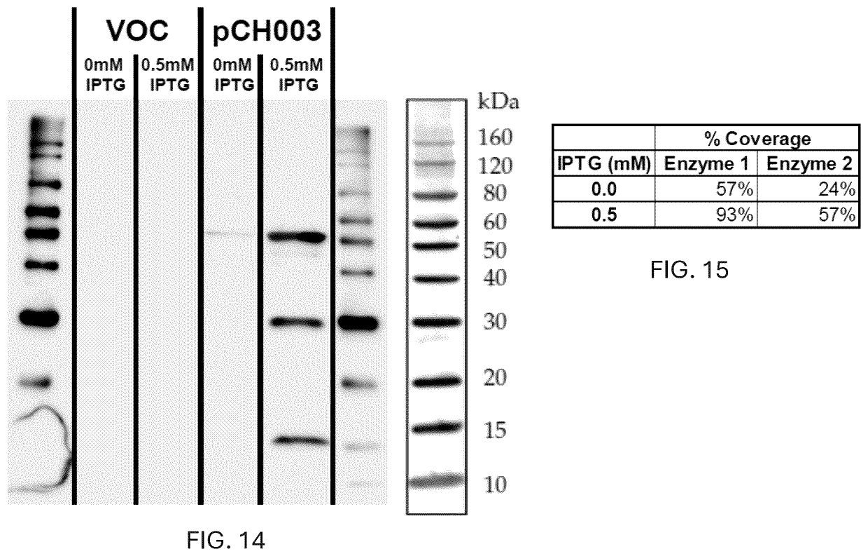

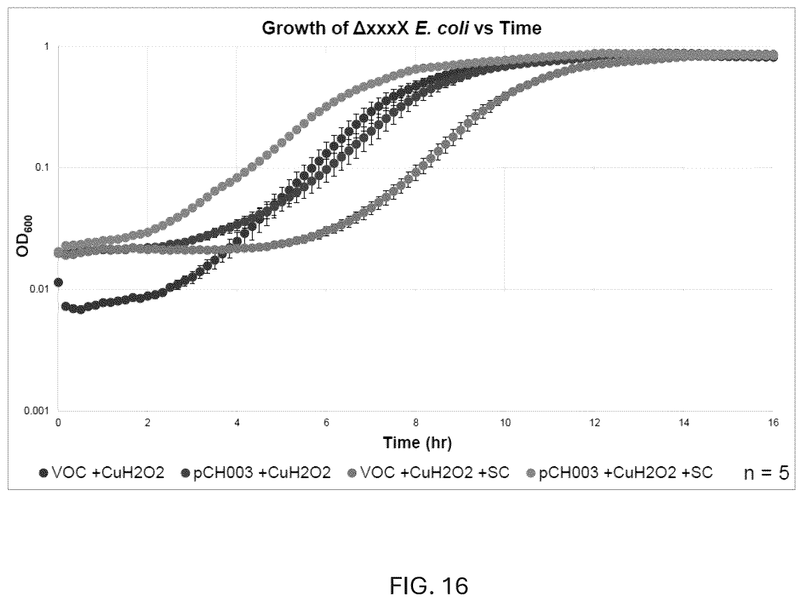

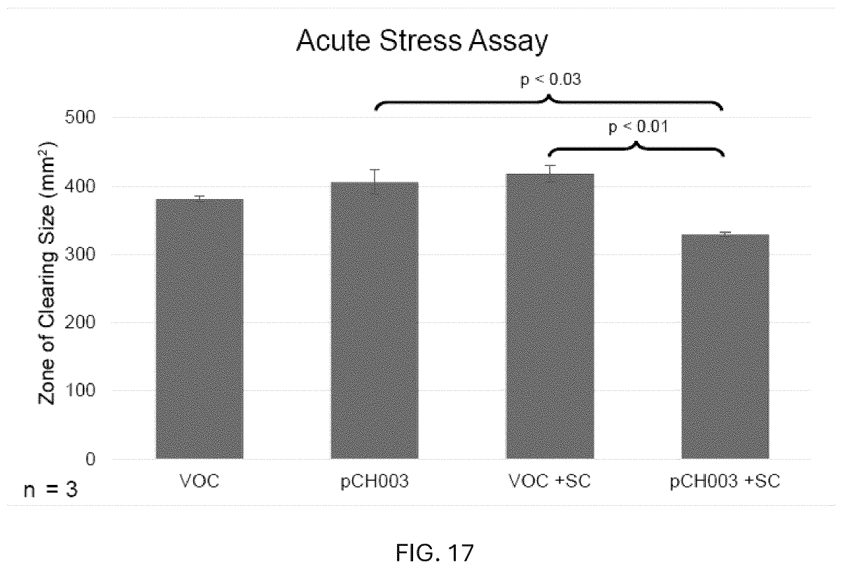

The inventors demonstrated in FIG. 11 that applying CoM to plants improves their growth and production of aerial tissues (stems, leaves, etc.) and increases their dry weight. Accordingly, methods of increasing aerial tissue growth in a plant or methods of increasing the dry weight of a plant are provided. In some embodiments, the methods comprise expressing the polynucleotides of the instant disclosure in the cells of the plant.

The aerial tissue growth or dry weight may be increased in comparison to a plant with cells that do not comprise the disclosed polynucleotides. The increase in growth or dry weight may be an increase relative to a control plant of 5%, 105, 15%, 20%, 25% or more.

The plant may be, e.g., Arabidopsis thaliana, Nicotinia tabacum (tobacco), Ocimum basilicum (basil), Cannabis sativa (cannabis), or Glycine max (soybean).

### Methods of Producing Coenzyme M

As discussed above, coenzyme M may be produced by chemical synthetic methods. However, the inventors discovered synthetic operons to bio synthetically produce coenzyme M. Accordingly, in an aspect of this disclosure methods of producing coenzyme M are provided. In some embodiments, the methods comprise culturing cells comprising the disclosed polynucleotides and harvesting the coenzyme M from the cultured cells.

Harvesting the CoM may comprise any suitable methods, e.g., lysing/homogenizing the cells and extracting, enriching, or purifying the CoM by known methods, e.g., liquid chromatograph (LC), high-performance LC (HPLC).

The cells being cultured may include, but are not limited to, prokaryotic cells, e.g., bacterial cells, or eukaryotic cells, e.g., yeast. The bacterial cells may be E. coli cells.

The polynucleotides may comprise one of SEQ ID NOs: 23-36 or sequences with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NOs: 23-36.

The inventors discovered that expressing the iron sulfur cluster (isc) operon improves expression of ComF, which comprises iron sulfur clusters as cofactors. Accordingly, the cells may further comprise at least one polynucleotide encoding at least one of scR, iscS, iscU, iscA, fdx, hscA, hscB, or iscX. The plasmid pDB1282 comprises each of the scR, iscS, iscU, iscA, fdx, hscA, hscB, and iscX sequences and is publicly available.

### Recombinant Archaeal Cells

The inventors discovered that expressing cysteate synthase and sulfopyruvate decarboxylase in the archaean Methanosarcina acetivorans increases the production of CoM and protectes the cells from oxidative damage by hydrogen peroxide. The archaeal cells may further comprise a deletion of HdrA1B1C1, which is encoded by hdrABC. The combination of exogenous cysteate synthase and sulfopyruvate decarboxylase expression and hdrABC deletion increases the archaeal cells tolerance to atmospheric oxygen and hydrogen peroxide. See Example 1.

### Additional Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the elements described herein in all possible variations thereof is encompassed by the invention unless otherwise indicated herein or otherwise clearly contradicted by context.

### EXEMPLARY EMBODIMENTS

1. A polynucleotide comprising a sequence encoding a coenzyme M synthase (ComF) operably linked to at least one heterologous regulatory element.
2. The polynucleotide of embodiment 1, wherein the ComF is Methanosarcina acetivorans ComF.
3. The polynucleotide of embodiment 1 or 2, wherein the ComF comprises SEQ ID NO: 1 or a sequence with at least 90% identity to SEQ ID NO: 1.
4. The polynucleotide of any one of the preceding embodiments, wherein the ComF comprises SEQ ID NO: 1.
5. The polynucleotide of embodiment 4, wherein the polynucleotide further comprises a sequence encoding a sulfoacetaldehyde-producing enzyme.
6. The polynucleotide of embodiment 5, wherein the sulfoacetaldehyde-producing enzyme comprises a taurine-pyruvate aminotransferase, a sulfoacetaldehyde acetyl transferase, or a sulfopyruvate decarboxylase.
7. The polynucleotide of embodiment 6 or 7, wherein the taurine-pyruvate aminotransferase comprises SEQ ID NO: 3 (sNB34) or a sequence with at least 90% identity to SEQ ID NO: 3 (Operon 3).
8. The polynucleotide of any one of embodiments 5-7, wherein the sulfoacetaldehyde-producing enzyme is located 5′ to the sequence encoding ComF.
9. The polynucleotide of any one of embodiments 5-8, wherein the heterologous regulatory element comprises a ribosome binding site and the sulfoacetaldehyde-producing enzyme is operably linked to a heterologous promoter.
10. The polynucleotide of any one of embodiments 5-9, wherein the promoter is located 5′ to the sulfoacetaldehyde-producing enzyme.
11. The polynucleotide of embodiment 6, wherein the sulfoacetaldehyde acetyl transferase comprises SEQ ID NO: 5 (sNB35) or a sequence with at least 80% identity to SEQ ID NO: 5.
12. The polynucleotide of embodiment 6, wherein the sulfopyruvate decarboxylase comprises SEQ ID NO: 6 (sNB33) or a sequence with at least 80% identity to SEQ ID NO: 6.
13. The polynucleotide of embodiment 12, wherein the polynucleotide further comprises at least one sequence encoding a D-3-phosphoglycerate dehydrogenase (sNB26), a phosphoserine aminotransferase (sNB27), a cysteate synthase (sNB28), an aspartate aminotransferase (sNB29), a phosphosulfolactate synthase (sNB30), a 2-phosphosulfolactate phosphatase (sNB31), a (2R)-3-sulfolactate dehydrogenase (sNB32), or a taurine-pyruvate aminotransferase (sNB34).
14. The polynucleotide of embodiment 12, wherein the polynucleotide further comprises sequences encoding a phosphosulfolactate synthase, a 2-phosphosulfolactate phosphatase, and a (2R)-3-sulfolactate dehydrogenase (Operon 1).
15. The polynucleotide of embodiment 12, wherein the polynucleotide further comprises a sequence encoding a cysteate synthase (Operon 2).
16. The polynucleotide of embodiment 12, wherein the polynucleotide further comprises a sequence encoding an aspartate aminotransferase (Operon 2.1).
17. The polynucleotide of embodiment 12, wherein the polynucleotide further comprises sequences encoding a cysteate synthase, an aspartate aminotransferase, a D-3-phosphoglycerate dehydrogenase, and a phosphoserine aminotransferase (Operon 6).
18. The polynucleotide of any one of embodiments 12-17, wherein
(i) the D-3-phosphoglycerate dehydrogenase comprises SEQ ID NO: 7, or a sequence with at least 90% identity to SEQ ID NO: 7 (sNB26);
(ii) the phosphoserine aminotransferase comprises SEQ ID NO: 8, or a sequence with at least 90% identity to SEQ ID NO: 8 (sNB27);
(iii) the cysteate synthase comprises SEQ ID NO: 9, or a sequence with at least 90% identity to SEQ ID NO: 9 (sNB28);
(iv) the aspartate aminotransferase comprises SEQ ID NO: 10, or a sequence with at least 90% identity to SEQ ID NO: 10 (sNB29);
(v) the phosphosulfolactate synthase comprises SEQ ID NO: 11, or a sequence with at least 90% identity to SEQ ID NO: 11 (sNB30);
(vi) the 2-phosphosulfolactate phosphatase comprises SEQ ID NO: 12, or a sequence with at least 90% identity to SEQ ID NO: 12 (sNB31);
(vii) the (2R)-3-sulfolactate dehydrogenase comprises SEQ ID NO: 13, or a sequence with at least 90% identity to SEQ ID NO: 13 (sNB32), or
(viii) the taurine-pyruvate aminotransferase comprises SEQ ID NO: 3, or a sequence with at least 90% identity to SEQ ID NO: 3 (sNB34).
   19. The polynucleotide of any one of the preceding embodiments, wherein the at least one heterologous regulatory element comprises a promoter or an enhancer.
   20. The polynucleotide of embodiment 19, wherein the promoter comprises a Cauliflower Mosaic Virus 35S (CaMV35S) promoter, Arabidopsis thaliana Act2 promoter, Oryza sativa Act-1 promoter, A. thaliana UBQ1 promoter, Panicum virgatum Ubi1 promoter, P. virgatum Ubi2 promoter, or Zea mays Ubi1 promoter.
   21. The polynucleotide of embodiment 19, wherein the promoter comprises a cytomegalovirus (CMV) promoter, an EF1a promoter, a CAG promoter, a phosphoglycerate kinase (PGK) promoter, tetracycline response element (TRE) promoter, Human U6 nuclear promoter (U6), and upstream activator sequence (UAS) promoter.
   22. A system comprising at least one polynucleotide comprising
(i) a sequence encoding a coenzyme M synthase (ComF); and
(ii) a sequence encoding a sulfoacetaldehyde-producing enzyme.
   23. The system of embodiment 22, wherein the system comprises at least two polynucleotides.
   24. The system of embodiment 22 or 23, wherein the ComF is Methanosarcina acetivorans ComF.
   25. The system of any one of embodiments 22-24, wherein the ComF comprises SEQ ID NO: 1 or a sequence with at least 80% identity to SEQ ID NO: 1 (sNB25).
   26. The system of any one of embodiments 22-25, wherein the sulfoacetaldehyde-producing enzyme comprises a taurine-pyruvate aminotransferase, a sulfoacetaldehyde acetyl transferase, or a sulfopyruvate decarboxylase.
   27. The system of embodiment 26, wherein the taurine-pyruvate aminotransferase comprises SEQ ID NO: 3 (sNB34) or a sequence with at least 90% identity to SEQ ID NO: 3.
   28. The system of embodiment 26, wherein the sulfoacetaldehyde acetyl transferase comprises SEQ ID NO: 5 (sNB35) or a sequence with at least 90% identity to SEQ ID NO: 5.
   29. The system of embodiment 26, wherein the sulfopyruvate decarboxylase comprises SEQ ID NO: 6 (sNB33) or a sequence with at least 90% identity to SEQ ID NO: 6.
   30. The system of embodiment 29, wherein the polynucleotide further comprises at least one sequence encoding a D-3-phosphoglycerate dehydrogenase (sNB26), a phosphoserine aminotransferase (sNB27), a cysteate synthase (sNB28), an aspartate aminotransferase (sNB29), a phosphosulfolactate synthase (sNB30), a 2-phosphosulfolactate phosphatase (sNB31), a (2R)-3-sulfolactate dehydrogenase (sNB32), or a taurine-pyruvate aminotransferase (sNB34).
   31. The system of embodiment 30, wherein the polynucleotide further comprises sequences encoding a phosphosulfolactate synthase, a 2-phosphosulfolactate phosphatase, and a (2R)-3-sulfolactate dehydrogenase (Operon 1).
   32. The system of embodiment 30, wherein the polynucleotide further comprises a sequence encoding a cysteate synthase (Operon 2).
   33. The system of embodiment 32, wherein the polynucleotide further comprises a sequence encoding an aspartate aminotransferase (Operon 2.1).
   34. The system of embodiment 30, wherein the polynucleotide further comprises sequences encoding a cysteate synthase, an aspartate aminotransferase, a D-3-phosphoglycerate dehydrogenase, and a phosphoserine aminotransferase (Operon 6).
   35. The system of any one of embodiments 30-34, wherein
(i) the D-3-phosphoglycerate dehydrogenase comprises SEQ ID NO: 7, or a sequence with at least 90% identity to SEQ ID NO: 7 (sNB26);
(ii) the phosphoserine aminotransferase comprises SEQ ID NO: 8, or a sequence with at least 90% identity to SEQ ID NO: 8 (sNB27);
(iii) the cysteate synthase comprises SEQ ID NO: 9, or a sequence with at least 90% identity to SEQ ID NO: 9 (sNB28);
(iv) the aspartate aminotransferase comprises SEQ ID NO: 10, or a sequence with at least 90% identity to SEQ ID NO: 10 (sNB29);
(v) the phosphosulfolactate synthase comprises SEQ ID NO: 11, or a sequence with at least 90% identity to SEQ ID NO: 11 (sNB30);
(vi) the 2-phosphosulfolactate phosphatase comprises SEQ ID NO: 12, or a sequence with at least 90% identity to SEQ ID NO: 12 (sNB31);
(vii) the (2R)-3-sulfolactate dehydrogenase comprises SEQ ID NO: 13, or a sequence with at least 90% identity to SEQ ID NO: 13 (sNB32), or
(viii) the taurine-pyruvate aminotransferase comprises SEQ ID NO: 3, or a sequence with at least 90% identity to SEQ ID NO: 3 (sNB34).
   36. The system of any one of embodiments 22-35, wherein the system comprises at least one regulatory element.
   37. The system of embodiment 36, wherein the at least one regulatory element comprises a promoter or an enhancer.
   38. The system of embodiment 37, wherein the promoter comprises a Cauliflower Mosaic Virus 35S (CaMV35S) promoter, Arabidopsis thaliana Act2 promoter, Oryza sativa Act-1 promoter, A. thaliana UBQ1 promoter, Panicum virgatum Ubi1 promoter, P. virgatum Ubi2 promoter, or Zea mays Ubi1 promoter.
   39. The system of embodiment 37, wherein the promoter comprises a cytomegalovirus (CMV) promoter, an EF1a promoter, a CAG promoter, a phosphoglycerate kinase (PGK) promoter, tetracycline response element (TRE) promoter (SEQ ID NO: 13), Human U6 nuclear promoter (U6) (SEQ ID NO: 14), and upstream activator sequence (UAS) promoter (SEQ ID NO: 15).
   40. A cell comprising the polynucleotide of any one of embodiments 1-21 or the system of any one of embodiments 22-39.
   41. The cell of embodiment 40, wherein the cell is a prokaryotic cell or a eukaryotic cell.
   42. The cell of embodiment 41, wherein the cell is a bacterial cell or a plant cell.
   43. The cell of embodiment 42, wherein the bacterial cell is an Escherichia coli cell.
   44. The cell of embodiment 42, wherein the plant cell is an Arabidopsis thaliana cell, a Nicotinia tabacum (tobacco) cell, an Ocimum basilicum (basil) cell, a Cannabis sativa (cannabis) cell, or a Glycine max (soybean) cell.
   45. A method comprising introducing the polynucleotide of any one of embodiments 1-21 into a cell.
   46. A method of increasing aerial tissue growth in a plant, the method comprising expressing the polynucleotide of any one of embodiments 1-21 in cells of the plant.
   47. A method of increasing dry weight of a plant comprising expressing the polynucleotide of any one of embodiments 1-21 in cells of the plant.
   48. The method of embodiment 46 or 47, wherein the plant is Arabidopsis thaliana, Nicotinia tabacum (tobacco), Ocimum basilicum (basil), Cannabis sativa (cannabis), or Glycine max (soybean).
   49. A method of producing coenzyme M (CoM), the method comprising culturing cells comprising the polynucleotide of any one of embodiments 1-21 and harvesting the CoM from the cultured cells.
   50. The method of embodiment 49, wherein the cells are prokaryotic cells.
   51. The method of embodiment 50, wherein the prokaryotic cells are bacterial cells.
   52. The method of embodiment 51, wherein the bacterial cells comprise Escherichia coli cells.
   53. The method of any one of embodiments 49-52, wherein the polynucleotide comprises at least one of SEQ ID NOs: 23-36, or at least one sequence with at least 90% identity to one of SEQ ID NOs: 23-36 (SEQ ID NOs: 23-33 are E. coli optimized nucleotide sequences and SEQ ID NOs: 34-36 are full plasmid sequences including the optimized Operon 3, or portions thereof).
   54. The method of any one of embodiments 49-53, wherein the cells further comprise at least one polynucleotide encoding at least one of scR, iscS, iscU, iscA, fdx, hscA, hscB, or iscX.
   55. The method of embodiment 54, wherein the at least one polynucleotide comprises or consists of pDB1282.
   56. A recombinant archaeal cell comprising the polynucleotide of embodiment 56.
   57. The recombinant archaeal cell of embodiment 57, wherein the archaeal cell is a Methanosarcina acetivorans cell.
   58. The recombinant archaeal cell of embodiment 57 or 58, wherein the recombinant archaeal cell has increased tolerance to hydrogen peroxide-induced stress as compared to an archaeal cell of the same species that does not comprise the polynucleotide.
   59. The recombinant archaeal cell of any one of embodiments 57-59, further comprising a deletion of HdrA1B1C1, which is encoded by hdrABC.
   60. The recombinant archaeal cell of embodiment 60, wherein the recombinant archaeal cell has increased tolerance to oxygen-induced stress as compared to an archaeal cell of the same species that does not comprise the polynucleotide.

### EXAMPLES

### Example 1—Overexpression of 2-Mercaptoethanesulfonate Biosynthesis Genes comDE Protects Methane-Producing Archaea from Oxidative Stress

Coenzyme M (2-mercaptoethane sulfonate, CoM) is an essential low molecular weight thiol in methanogenic archaea (methanogens) that serves as a methyl carrier and as a component of CoM-S—S-CoB heterodisulfide comprised of CoM and coenzyme B (7-mercaptoheptanoylthreoninephosphate) which serves as the terminal electron acceptor in methanogenesis. Increasing CoM in Methanosarcina acetivorans cells by overexpressing biosynthesis genes results in faster growth on methanol or methanol+acetate medium in the absence of sulfide. Furthermore, CoM overproduction enhances resistance to oxidative stress in the ΔhdrABC mutant genetic background. The ΔhdrABC mutant is resistant to 5% O₂ atmosphere, and overexpression of com⁺ genes resulted in resistance to up to 2 mM hydrogen peroxide in a stress assay. Increased resistance to oxidative stress is correlated with 45.7% higher levels of intracellular total CoM, and 74.7% higher ratio of reduced CoM-SH to total CoM in the parent versus the ΔhdrABC com⁺ mutant strain. Our study suggests increased expression of genes encoding coenzyme M biosynthesis, in conjunction with deletion of the nonessential heterodisulfide reductase HdrABC, increases oxidative stress resistance of the strictly anaerobic methanogen Methanosarcina acetivorans.

### Importance

Methanogenic archaea (methanogens) are key organisms in the global carbon cycle that are harnessed to produce renewable methane for energy and transportation fuel. Methanogens are strict anaerobes commonly found in subsurface sediment, anaerobic digesters, and digestive tracts of animals such as the rumen. Our results suggest methanogens have the genetic and biochemical potential to adapt to prolonged exposure to oxidative stress under the appropriate environmental conditions, and it may be possible to engineer redox homeostasis in methanogens. Engineering redox homeostasis in methanogens and other strict anaerobes has potential to reduce technical barriers to culturing, thus accelerating research progress on a wide variety of non-model microbes, and ultimately broadening potential biotechnology applications related to sustainable food, fuel, and biomedical uses.

### Introduction

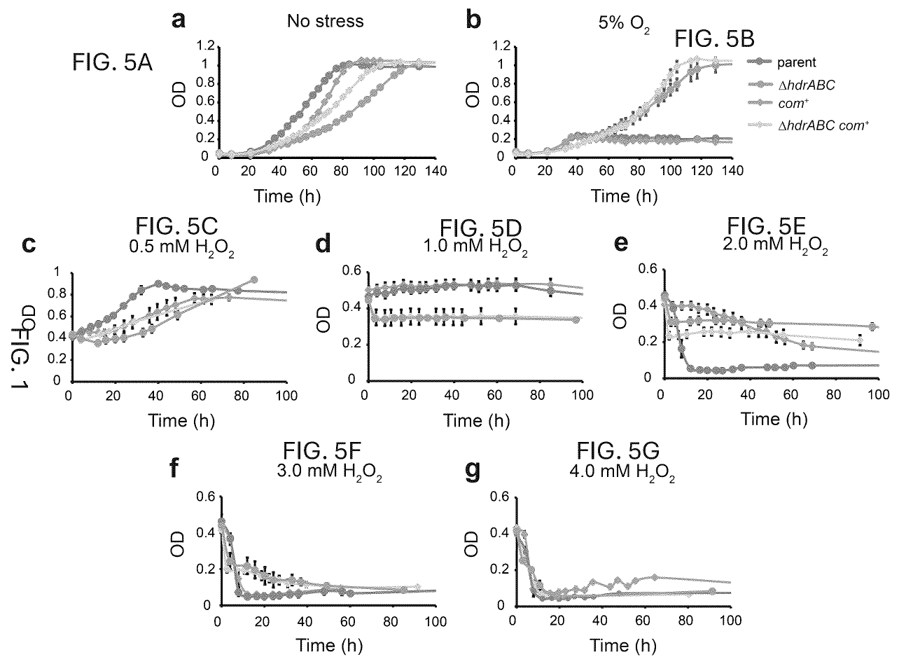

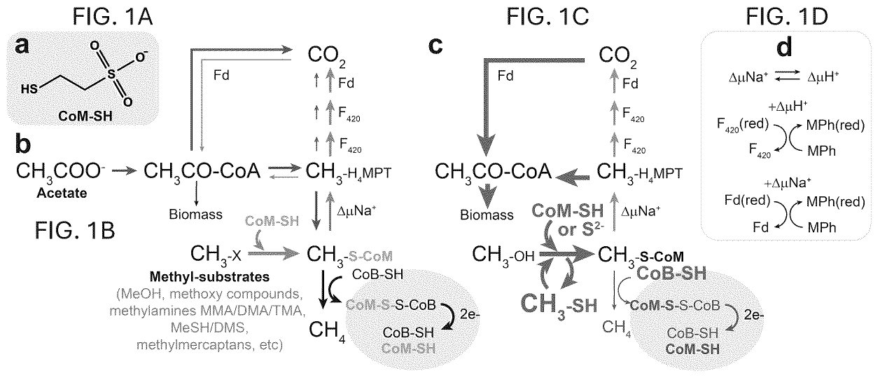

Methanogens are organisms that grow by producing methane gas via the Wolfe cycle of methanogenesis (FIG. 1). They thrive in a variety of anaerobic habitats such as in the deep ocean or subsurface sediment, in digestive tracts of insects and animals, and in anaerobic digesters. They dominate in anaerobic environments where sulfate or other more thermodynamically favorable terminal electron acceptors (such as sulfate) are absent. It is estimated that globally, methanogens contribute 2 gigatons of methane annually to the global carbon cycle and thus play an important role in biogeochemical nutrient cycling and climate.¹

Methanosarcina acetivorans was originally isolated from marine sediment² and is an emerging model for exploring the biotechnology potential of methanogens to produce renewable fuels and chemicals from inexpensive non-food feedstocks (for example, methane, CO₂, CO, formate, methanol, etc.) via the Wolfe Cycle.³⁴ M. acetivorans can naturally grow on methylotrophic substrates (such as methanol, methylamines, methylsulfides), carbon monoxide or acetate. Recently, M. acetivorans has been engineered to increase the rate of methanogenesis,⁵⁻⁶ produce high yields of isoprene,⁷ has been converted into an acetogen⁸ or to grow in the reverse methanotrophic direction,^{9, 10} and can participate in interspecies electron transfer.¹¹ Enhancement of these processes through genetic selection and/or genetic engineering requires a detailed understanding of how intracellular redox homeostasis is maintained to ensure efficient functioning of metabolism under changing process conditions. A central molecule in methanogenesis and redox homeostasis is 2-mercaptoethanesulfonate, Coenzyme M (CoM).

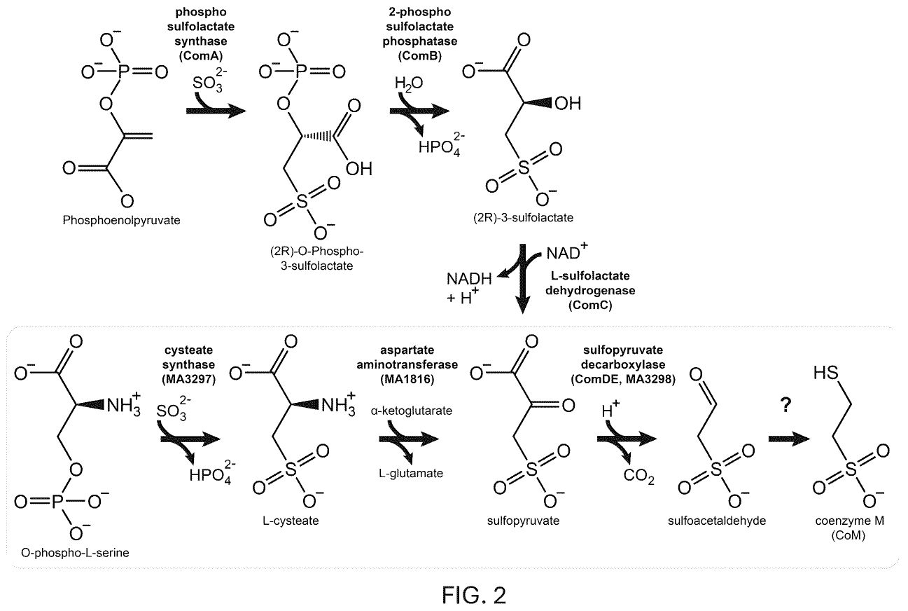

Coenzyme M is the smallest coenzyme discovered to date. It is essential in methanogens, where it acts as a methyl carrier that accepts methyl groups from corrinoid methyltransferases to produce CH₃—S-CoM. It is also a component of the CoM-S—S-CoB heterodisulfide formed from coenzyme M and coenzyme B (7-mercaptoheptanoyl threonine phosphate) which serves as the terminal electron acceptor in the methanogenic energy conservation pathway, (FIG. 1).^{12, 13} CoM is synthesized by at least two pathways in methanogens (FIG. 2).¹⁴ In Methanococcales, Methanobacteriales, and Methanopyrales, phosphoenolpyruvate is converted to sulfoacetaldehyde by ComABCD/E enzymes, while Methanosarcinales and Methanomicrobiales instead synthesize sulfoacetaldehyde from L-phosphoserine using cysteate synthase (MA3297), an general aspartate aminotransferase (aspAT), and sufopyruvate decarboxylase, comDE (MA3298). The last step in the pathway is addition of sulfur and reduction of sulfoacetaldehyde to form CoM in what is thought to be a non-enzymatic reaction.

In previous work it was observed that when the genes encoding the methylotrophic-specific HdrA1B1C1 enzyme (HdrABC) was deleted cells were still viable, but ¹³C NMR and transcriptomic studies suggested the ΔhdrABC mutant phenotype was caused by decreased ferredoxin redox cycling and changes in coenzyme M (CoM-SH) homeostasis.¹⁵ Hdr enzyme is necessary to reduce CoM-S—S-CoB to regenerate CoM-SH and CoB-SH thiols for subsequent rounds of methanogenesis. Hdr comes in two versions in Methanosarcina, an essential membrane-bound cytochrome-containing HdrED that conserves energy, and a soluble HdrABC that uses electrons from ferredoxin and/or reduced cofactor F420 to reduce CoM-S—S-CoB. HdrA1B1C1 is cotranslated from a single operon and is specific to methylotrophic substrates, while HdrA2C2B2 is essential although preferentially expressed during aceticlastic methanogenesis and is transcribed from two operons, HdrA2:polyferredoxin and HdrC2B2.¹⁵ Deletion of genes encoding the methylotrophic HdrABC resulted in upregulation of methyltransferases, carbon monoxide dehydrogenase CdhA2, sulfonate transporters, and genes proposed to be involved in CoB-SH synthesis (2-isopropylmalate synthase, MA4615) and CoM-SH synthesis (cysteate synthase and sulfopyruvate decarboxylase comDE, MA3297-3298).¹⁵ These data were interpreted to suggest that in the absence of HdrABC, reduction of CoM-S—S-CoB is slowed, resulting in slower uptake of substrate by methyltransferases, resulting in production of methane thiol (MeSH) and dimethylsulfide (DMS) which can ultimately be used as substrates (FIG. 1c). As a result, while kinetics of methanogenesis is decreased, CoM-SH flux increases to compensate, and metabolic efficiency is increased.^{5, 15} This model was further supported when it was shown that adding sulfide or supplementing cultures with acetate and exogenous CoM-SH can also partially rescue the zhdrABC mutant growth defect.¹⁶ These findings suggest that intracellular CoM-SH pools and redox homeostasis can be altered in M. acetivorans. Therefore, we wanted to test if directly increasing the intracellular CoM-SH pool in cells by overexpressing the com locus (MA3296-MA3298) can affect growth and redox homeostasis.

### Materials and Methods

Culture conditions. Organisms were obtained from the sources listed in Table 1. Methanogens were grown in high salt mineral medium (HS) [200 mM NaCl, 45 mM NaHCO₃, 13 mM KCl, 54 mM MgCl₂·6H₂O, 2 mM CaCl₂·2H₂O, 2 μM 0.1% resazurin (w v⁻¹), 5 mM KH₂PO₄, 19 mM NH₄Cl, 2.8 mM cysteine·HCl, 0.1 mM Na₂S·9H₂O, trace elements, vitamin solution] as described¹⁷ and supplemented with a carbon and energy source (methanol, 125 mM; trimethylamine, 50 mM; sodium acetate, 120 mM) and 2 mg L⁻¹ puromycin as needed at 35° C. For growth on solid media, 1.4% agar was added to HS media. Methanogens were grown anaerobically in a custom B-type Coy anoxic chamber (Coy Labs, Grass Lake, MI) under a 5% H₂/20% CO₂/75% N₂ (±3%) (Matheson Gas, Lincoln, NE) atmosphere. Cells incubated outside of anaerobic chamber are contained in glass Balch tubes secured with butyl rubber stoppers (Bellco Glass, Vineland, NJ) and aluminum crimps (Wheaton, Millville, NJ).

Escherichia coli cells were grown aerobically in 0.5% glucose Lysis Broth (LB)¹⁸ with with shaking at 37° C. with supplementation as appropriate: 0.5% agar, rhamnose (1 mM), chloramphenicol, 10-35 μg ml⁻¹). Chemicals and reagents were sourced from Millipore Sigma (St. Louis, MO) or Fisher Scientific (Waltham, MA).

Culture growth was measured using a Spectronic D spectrophotometer (ThermoFisher, Waltham, MA) fitted with a Balch tube (18 mm) modification or using a Tecan Sunrise UV/Vis spectrophotometric plate reader (Tecan, Männedorf, Switzerland).

Plasmid cloning, strain construction and validation. Plasmids and primers shown in Table 1 were designed using VectorNTI software (ThermoScientific, Waltham MA). PCR primers were synthesized by Integrated DNA Technologies (IDT, Coralville, IA). The proofreading Phusion Flash PCR Master Mix was used for all PCR amplification (ThermoScientific, Waltham, MA). Promega Wizard SV Gel and PCR Clean-up kits (Madison, WI) were used for DNA purification. Fast Digest Restriction Enzymes (BamH1 and Ndel) were purchased from ThermoScientific (Waltham, MA). AscI was purchased from NEB (Ipswich, MA). DNA fragments were assembled using the Sequence and Ligation Independent Cloning (SLIC) protocol previously described.¹⁹ Two promoters, P_{mcr} and Pₜₑₜ, were used to test whether promoter strength affected the observed phenotypes. P_{mcr} is a strong constitutive promoter. Pₜₑₜ is identical to P_{mcr} except it contains a tetO1 TetR repressor binding site, resulting in lower constitutive expression in tetR-deficient strains. All plasmid inserts were verified by sequencing (Eurofins, Louisville, KY).

After growth curves, strain genotypes were confirmed using a PCR assay as previously described using the primers listed in Table 1.^{5, 20}

Oxidative stress assays. Oxidative stress assays were carried out as described^{21, 22} after adapting M. acetivorans to HS methanol medium without resazurin or sodium sulfide for 15 generations (3 passages of 0.25 ml into 10 ml cultures). For O₂ stress assays cells were grown to OD_{600 nm} of 0.4 whereupon cultures were injected with either sterile 100% O₂ gas (Matheson) to 1% or 5% v/v/headspace at 1 atm, ambient air (20% O₂/80% N₂) at 1 atm, or freshly obtained H₂O₂ at 1.5 mM or 3 mM final concentration.

Thiol extraction and quantification. M. acetivorans was grown in 10 mL HS media with MeOH as carbon source at 35° C. until OD₆₀₀=0.5-0.6 (mid exponential). Cultures were centrifuged anaerobically and cells were washed twice with 0.85M bicarbonate-buffered sucrose. Cells were lysed by osmotic shock by resuspension in 1 ml anaerobic H₂O and low-molecular weight thiols were extracted and derivatized with monobromobomane (mBBr). The derivatization of thiol compounds with monobromobimane (mBBr) was modified based on published methods.^{23, 24} Because of its small size and high electronegativity, CoM-SH must be derivatized to allow quantification. However, because it is a reactive thiol, CoM-SH may become oxidized in the cell due to metabolism or inadvertently during extraction, and a portion of the CoM sample may be in the reduced CoM-S—R form where “R” represents any of the following: CoM-S—CH₃, CoM-S—S-CoM, CoM-S—S-CoB, CoM-S-Cys (free cysteine or a protein thiol), CoM-S—Fe(II)/S, CoM-S—Co(II)rrinoid, CoM-S—Fe(II)heme, CoM-S—Ni(II)F430. To quantity free CoM-SH vs total CoM (CoM-SH+CoM-S—R), matched samples were split: one was derivatized with mBBR directly to quantify free CoM-SH, while the other matched sample was reduced with KBH₄ before derivatization with mBBr to quantify total CoM (CoM-SH+CoM-S—R). The concentration of reduced CoM-S—R was calculated by subtracting the CoM-SH amount from the total CoM measured after KBH₄ reduction. Briefly, after removing an aliquot for protein quantification (Bradford), half the extracts were reduced with 92.6 mM KBH₄ while the other half was diluted with H₂O. Unreacted KBH₄ was quenched with 2.5 ml 1M HCl followed by 2.5 ml 1M NaOH. Samples were diluted with 615 ml buffer (200 mM HEPES, 5 mM diethylenetriamine pentaacetate (DTPA) pH 8.2) reacted with 10 ml 20 mM mBBr in acetonitrile in the dark for 30 minutes. The derivatization reaction was quenched with 100 ml methanesulfonic acid. Derivatized thiols were quantified by reverse-phase high-pressure liquid chromatography (HPLC) using a Dionex UltiMate 3000 HPLC with diode array and fluorescence detection (Thermo Scientific, Waltham, MA). Samples were injected onto a Supelcosil LC-18 15 cm by 4.6 cm, 5 μm column fitted with a SupelGuard C18 guard column (Sigma-Aldrich). Analytes were separated by gradient from 10% ACN, 0.1% TFA (v/v) in H₂O to 99% ACN, 0.1% TFA (v/v) mobile phase at 1 mL min⁻¹ and washed with 100% methanol at 2.5 mL min⁻¹.²⁵ mBBr was followed by 380 nm (ex) and 470 nm (em). Peak areas were normalized to soluble protein concentration and quantified by comparison to standards: cysteine (Fisher), coenzyme M (Fisher), and coenzyme B (Buan Lab stock synthesized as described).^{26, 27}

### Results

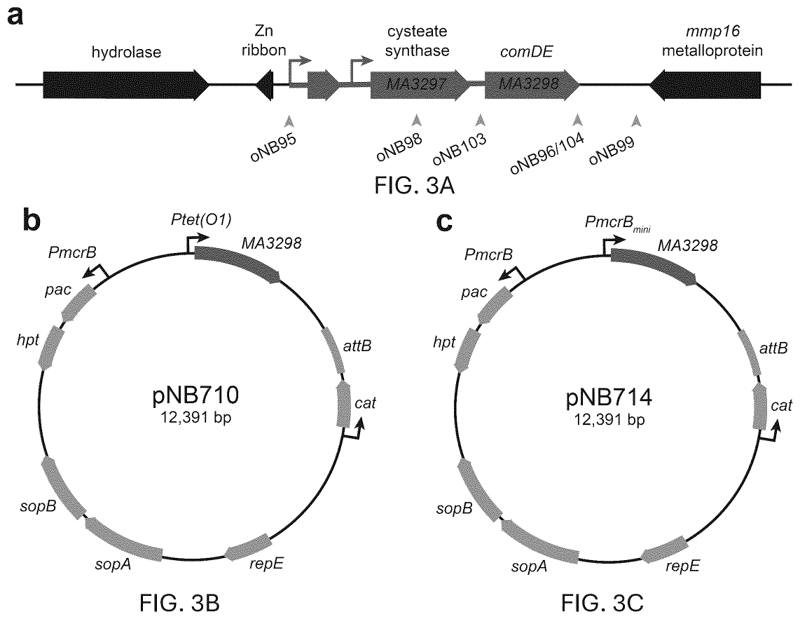

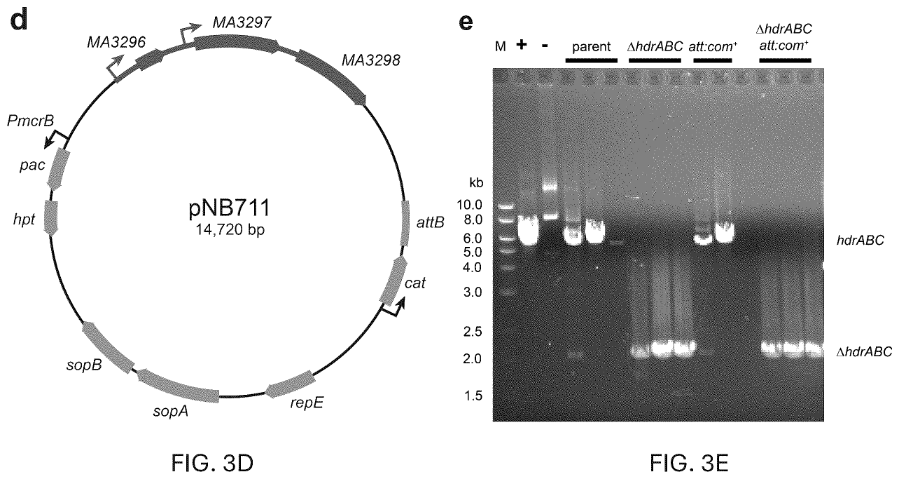

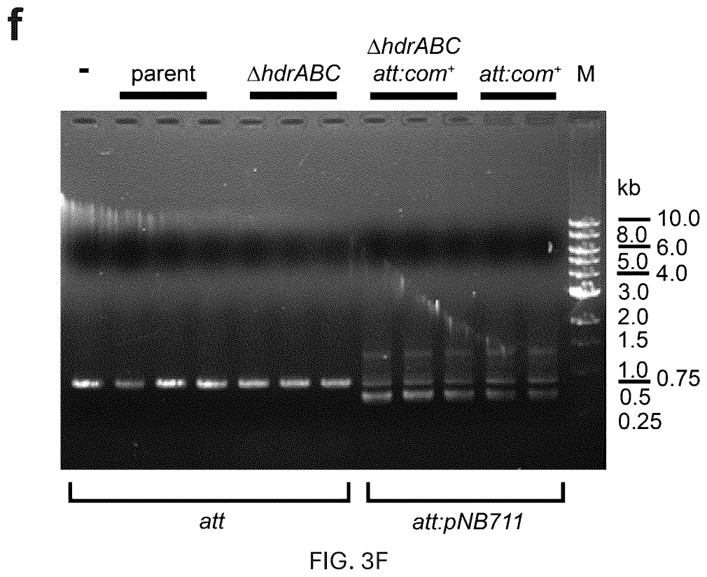

Construction of com⁺ overexpression strains. To uncouple CoM-SH synthesis from direct or indirect effects of the ΔhdrABC mutation, we synthesized several plasmids in an attempt to influence intracellular CoM-SH levels. Unfortunately, the CoM-SH biosynthetic pathway is not fully understood in M. acetivorans, and it seems the pathway differs from other methanogens as no clear homologs for several steps in the pathway are identified. Therefore, we focused on the two genes which appear to be upregulated in the ΔhdrABC mutant, MA3297 (cysteate synthase) and MA3298 (comDE) (FIG. 3a). We cloned MA3298, encoding comDE, into pJK026A and pJK027A resulting in plasmids pNB710 (FIG. 3b) and pNB714 (FIG. 3c), respectively. pNB710 expresses comDE from a Ptet(01) promoter (medium-strength), and pNB714 expresses comDE from the highly-expressed constitutive PmcrBₘᵢₙᵢ promoter. We also replaced the promoter of pJK027A with the entire MA3296-MA3298 operon and upstream promoter region to produce pNB711 (FIG. 3d). All three plasmids contain ϕC31 attB sites to integrate onto the parent strain chromosome. Each plasmid was transformed into the parent strain and the ΔhdrABC mutant, where genotypes were confirmed using PCR screens (FIG. 3ef) using primers listed in Table 1.

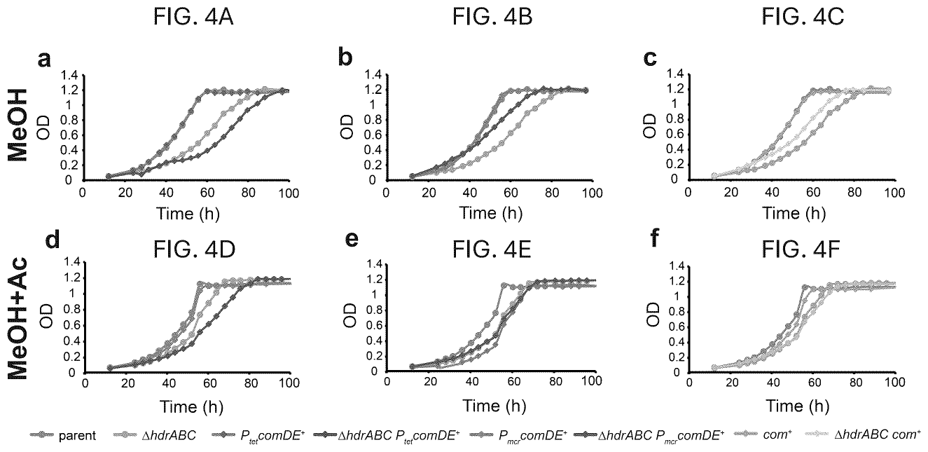

Overexpression of PₜₑₜcomDE⁺ improves growth on methanol and methanol+acetate as energy sources. Strains were grown on methanol or methanol+acetate as energy sources to determine if overexpression of com genes affected growth during methylotrophic or mixotrophic growth. Growth on acetate as sole energy source was not tested, because previous work showed the parent and DhdrABC mutant strains had the same growth rates on this substrate. On methanol, overexpression of P_{mcr}comDE⁺ resulted in a slightly decreased population doubling time to 8.97 h (±0.233) versus the parent at 10.27 h (±0.518) (Table 3). None of the plasmids affected growth rates of the AhdrABC mutant on methanol, however, differences in lag times were observed (FIG. 4a-c). Mixotrophic growth on methanol+acetate as energy sources was tested (FIG. 4d-f), as previous research showed addition of exogenous CoM-SH under these conditions does not affect the parent strain but is able to rescue the growth rate defect of the AhdrABC mutant strain.¹⁶ Under mixotrophic conditions, both the P_{mcr}comDE⁺ and the com⁺ overexpression plasmids resulted in faster growth rate when transformed into the parent strain (8.22 h±0.196 and 9.68 h±0.323 versus 10.81 h±0.139, respectively). None of the overexpression plasmids affected the growth rate of the AhdrABC mutant, indicating that overexpression of comDE alone or of the entire com locus was not capable of producing enough CoM-SH to overcome the lack of HdrABC.

Overproduction of CoM results in increased resistance to oxidative stress. As a low molecular weight thiol, CoM-SH is often compared to glutathione (GSH) as both are present at ˜3 mM in methanogen and bacterial or eukaryal cells, respectively.^{28, 29} Because methanogens do not synthesize GSH,³⁰ it has been hypothesized that perhaps CoM-SH, in addition to playing its vital roles in methanogenesis (FIG. 1), may also be involved in resistance to oxidative stress by virtue of its inherent chemical properties as a thiol molecule. Recently, increased CoM-SH production was observed to occur as a result of adaptation to exposure to oxygen and heavy metals.^{31, 32} We wanted to test whether the strains we generated may also be tolerant to oxidative stress.

The parent, AhdrABC, P_{mcr}comDE⁺, and com⁺ strains were adapted to methanol medium without added sulfide which serves as a chemical antioxidant. Under these conditions, without stress (FIG. 5a), we observed slightly increased growth rates of the P_{mcr}comDE⁺, and com⁺ strains in both the parent and AhdrABC genetic backgrounds (Table 4). Under O₂ stress, in which cultures are inoculated into tubes with a headspace atmosphere containing 5% O₂, we were surprised to observe the AhdrABC and AhdrABC com⁺ strains are very resistant and are ultimately able to reach full culture density (FIG. 5b). Under these stress conditions, the AhdrABC mutant had a doubling time of 26.14 h (±3.147) and the AhdrABC com⁺ mutant had a doubling time of 15.30 h (±1.713). In contrast, the parent and com⁺ strains did not achieve an OD higher than 0.2. These results are interpreted to suggest that the AhdrABC mutation results in changes to cellular physiology that allows cells to detoxify molecular oxygen, and that overexpression of com genes enhances this effect. Possible mechanisms include by increased expression of corrinoid proteins, which are highly sensitive to oxidation and could theoretically directly scavenge oxidants, requiring ATP-dependent repair by the ram system.^{33, 34} Alternatively, corrinoid proteins may nonspecifically react with CoM-S—S-CoM disulfide forming CoM-SH and CoM-S-corrinoid adducts (which may be regenerated by corrinoid methyltransferases), thus increasing overall CoM-SH turnover, as overexpression of the com genes alone in the parent background is not sufficient to result in detectable resistance to O₂.

We next tested whether the parent, AhdrABC, com⁺, and AhdrABC com⁺ strains are resistant to H₂O₂ exposure. In these experiments, cells were grown to mid-exponential phase (OD=0.4) and dosed with increasing levels of hydrogen peroxide from 0.5 to 4 mM (FIG. 5c-g). At 1.0 mM H₂O₂, the parent and com⁺ strains cease growth, while cultures of AhdrABC and AhdrABC com⁺ strains showed a slight decrease in optical density and then remain static at a lower final optical density than the parent and com⁺ strains. Surprisingly, at 2.0 mM H₂O₂, the parent strain rapidly lysed within 12 hours, while the AhdrABC, com⁺, and AhdrABC com⁺ strains were resistant to lysis. By 4.0 mM H₂O₂, cells from all four strains were sensitive to killing. These results indicate that while the AhdrABC mutant is resistant to molecular oxygen and exposure to H₂O₂, the com⁺ overexpression plasmid confers increased resistance to H₂O₂ in both the parent and AhdrABC genetic backgrounds.

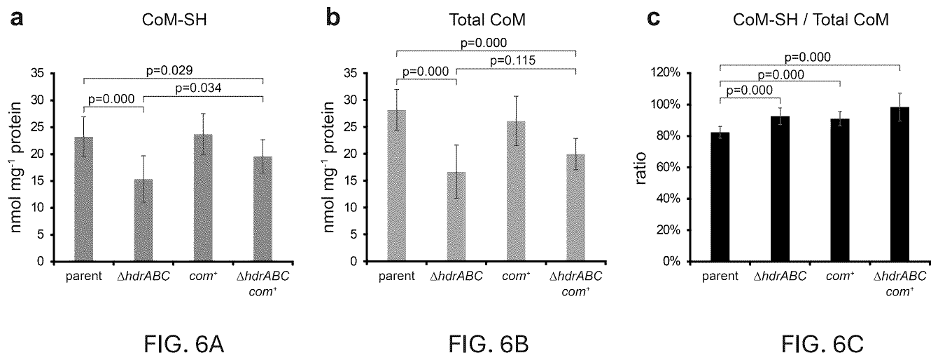

Deletion of AhdrABC and overexpression of com results in increased proportion of reduced CoM-SH. To test whether resistance to oxidative stress is correlated to increased intracellular levels of CoM-SH, we extracted and measured CoM-SH levels in parent, ΔhdrABC, com⁺, and ΔhdrABC com⁺ strains (FIG. 6, Table 5). The concentration of free CoM-SH was estimated to be 23.24 nmol mg⁻¹ protein (±3.70) in the parent strain, 15.35 nmol mg⁻¹ protein (±4.33) in the zhdrABC mutant, 23.69 nmol mg⁻¹ protein (±3.82) in the com⁺ mutant, and 19.58 nmol mg⁻¹ protein (±3.10) zhdrABC com⁺ strain (FIG. 6a). Total CoM was derived from the aliquots which were reduced using KBH₄ prior to derivatization with mBBr, this quantity non-exhaustively includes CoM-SH, CH3-CoM, CoM homodisulfides, and CoM heterodisulfides. The concentration of total CoM was estimated to be 28.16 nmol mg⁻¹ protein (±3.81) in the parent strain, 16.66 nmol mg⁻¹ protein (±4.97) in the ΔhdrABC mutant, 26.11 nmol mg⁻¹ protein (±4.59) in the com⁺ mutant, and 19.95 nmol mg⁻¹ protein (±2.91) ΔhdrABC com⁺ strain (FIG. 6b). We noted the ΔhdrABC strain has a 66% decrease in the amount of free CoM-SH in cell extracts versus the parent strain, confirming previous work which suggested CoM was limiting in the ΔhdrABC strain.¹⁵ The decreased amount of free CoM-SH and total CoM was complemented by com⁺ expression, confirming a role for MA3297-3298 in CoM biosynthesis (FIG. 6ab). However, com⁺ expression in the parent strain did not increase free CoM-SH or total CoM levels, suggesting that com gene dosage is not limiting in the parent genetic background. Expression of com⁺ resulted in higher ratio of CoM-SH to CoM-S—R (oxidized CoM which includes CoM-S—S—B, CoM-S—S-CoM, CoM-S—S-Cys as free amino acid or on a protein, CoM-S—Fe(II)/S, CoM-S—Co(II)rrinoid, CoM-S—Fe(II)heme, or CoM-S—Ni(II)F430 that may be reduced by borohydride, <−1.24V vs SHE) when expressed from the parent background (10.55% increase, p=0.000) and from the ΔhdrABC background 6.28% fold increase, p=0.058) (FIG. 6c).

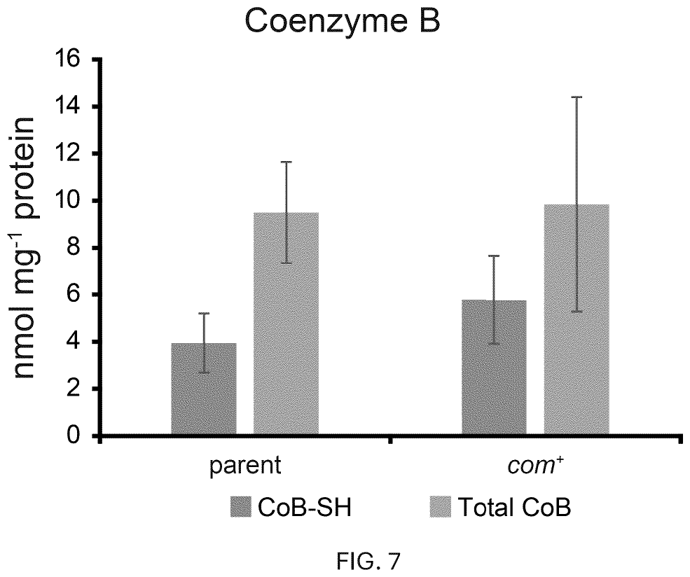

We were also able to detect CoB in cell extracts (FIG. 7, Table 5). The intracellular concentration of total CoB was estimated to be 1.2 mM (±0.36) in the parent strain, 0.10 mM (±0.01, p=0.003) in the ΔhdrABC mutant, 0.63 mM (±0.24, p=0.022) in the com⁺ mutant, and 0.12 mM (±0.03, p=0.003) ΔhdrABC com⁺ strain. The ΔhdrABC strain has a 49% decrease in free CoB-SH (p=0.174) and only 6.3% total CoB vs parent strain (p=0.003) (FIG. 7). Expression of com⁺ in the parent or ΔhdrABC strains does not increase CoB levels. When comparing the ratio of free CoB-SH in cell extracts versus total CoB obtained after chemical reduction, CoB is primarily in the oxidized state in the parent (75%) and com⁺ (62%) strains (most likely CoM-S—S-CoB), whereas the ΔhdrABC and ΔhdrABC com⁺ strains have virtually all of their CoB in the reduced CoB-SH state (90%±10.1 and 85%±4.3, respectively) (FIG. 7) indicating M. acetivorans has significantly higher levels of reduced CoB-SH versus oxidized CoB in the cell (CoB-S—R, CoM-S—S-CoB, CoB-S—S-CoB). The ratio of total CoM to total CoB was 2.2 (±0.87) in the parent strain, 16.8 (±7.48, p=0.000) in the zhdrABC mutant, 3.3 (±0.88, p=0.088) in the com⁺ mutant, and 16.2 (±6.99, p=0.000) zhdrABC com⁺ strain.

Overall, we observed that the zhdrABC, com⁺, and zhdrABC com⁺ mutant strains have significantly more reduced CoM-SH intracellular pools than the parent strain. The increased proportion of free CoM-SH in zhdrABC, com⁺ and zhdrABC com⁺ strains correlates with resistance to H₂O₂ stress, congruent with its role as an antioxidant The decrease of total CoM in zhdrABC and zhdrABC com⁺ strains correlates with their increased doubling times while the increase of CoM-SH and total CoM between the two strains confirms that com⁺ improves CoM production. The absence of increased CoM production in the com⁺ strain indicates that com⁺ alone is insufficient in increasing CoM production in the parent.

### Discussion

Our results support the hypothesis that in addition to serving essential roles as C-1 carrier and part of the terminal electron acceptor, CoM-SH can function as a scavenging antioxidant in Methanosarcina. Unlike most bacteria, eukarya, or archaea, methanogens do not produce glutathione or gamma-glutamyl cysteine to use as a general protectant from oxidative stress.³⁰ However, methanogens synthesize high levels of coenzyme M which has potential to act as a general antioxidant similar to glutathione. We observed deletion of AhdrABC protects cells from molecular oxygen, and overproduction of CoM-SH protects cells from H₂O₂. When combined, the ΔhdrABC com⁺ strain can withstand exposure to 5% O₂ or up to 2 mM H₂O₂ in oxidative stress, which is highly tolerant to oxidative stress relative to other anaerobes.³⁵

Molecular oxygen readily oxidizes flavins or labile organometallic cofactors which are abundant in methanogens such as in heme cytochromes, coenzyme Ni(I)F430 in methyl coenzyme M reductase, Co(I/II)rrinoids in methyltransferases, and iron-sulfur clusters, resulting in generation of superoxide radical and inactivation of enzymes, loss of Fe(II) from Fes/S clusters and subsequent damage to proteins, DNA, and lipid membranes.³⁶ Superoxide is converted to H₂O₂ by superoxide dismutase (sod), of which there are two homologs in M. acetivorans (MA_RS08180/MA1574, and MA_RS12570/MA2422) through a GSH-independent process. H₂O₂ can then react with organometallic cofactors or free Fe(II) by Fenton chemistry to produce peroxide radicals, which readily react with sulfhydryls and iron-sulfur clusters, thus exposure to O₂ or H₂O₂ affects many enzymes involved in central metabolism. Cells may use catalase, in the form of katE or the bifunctional katG catalase/peroxidase to convert H₂O₂ to water and O₂, or anaerobic microbes may employ alternative cytochrome or rubredoxin systems to detoxify O₂ or repair peroxide damage without producing O₂ as a byproduct.³⁵

Previous investigators noted M. acetivorans has an inactivating frameshift in katE (MA2081) but does seem to have an intact katG (MA0972).²² Although cells do not have constitutive or inducible catalase activity, expression of E. coli katG (with heme supplementation), conferred increased resistance to H₂O₂ but not to O₂.²² After prolonged selection with O₂, M. acetivorans evolved resistance up to 2% O₂, which correlated with increased expression of stress resistance genes (sod, katG, and alkyl peroxidase apx) and higher intracellular amounts of cysteine, CoM-SH, sulfide, and polyphosphate.³¹ We observed roughly equivalent resistance in the H₂O₂ and O₂ stress assays for the ΔhdrABC and ΔhdrABC com⁺ strains as when E. coli katG is introduced but without induction of sod, katG, or other predicted peroxidases,¹⁵ and the ΔhdrABC com⁺ strain did not accumulate cysteine or sulfide, and did not show a tendency to form biofilms.³¹

We interpret these observations to suggest increased turnover of CoM and increased expression of corrinoid methyltransferases,¹⁵ especially with overexpression of com genes may help scavenge reactive oxygen species to protect the cell from damage. These results also suggest that M. acetivorans could potentially be selected or engineered to resist higher levels of oxidative stress, for instance by combining ΔhdrABC com⁺ mutations with enhanced katG expression, upregulating peroxidases and cytochromes, or exploring fermentation process and stress conditions similar to approaches taken by others to enhance GSH production in yeast.³⁷ Air-tolerant methanogens such as these could be desirable to enhance renewable biogas production or may enable using M. acetivorans for inexpensive synthesis of a wider array of chemicals that require molecular oxygen for biocatalysis such as taxol (paclitaxel).³⁸⁻³⁹

TABLE 1
Primers, plasmids, and strains used in this study.

| Primers | | | |
|---|---|---|---|
| Name | Sequence | Purpose | Source |
| oNB52 | GAAGCTTCCCCTTGACCAAT (SEQ | ϕC31 screen-all#1; Validation | 20 |
| | ID NO: 50) | of plasmid integration | |
| oNB53 | TTGATTCGGATACCCTGAGC (SEQ | ϕC31 screen-C2A#1; | 20 |
| | ID NO: 51) | Validation of plasmid | |
| | | integration | |
| oNB54 | GCAAAGAAAAGCCAGTATGGA | ϕC31 screen-pJK200#1; | 20 |
| | (SEQ ID NO: 52) | Validation of plasmid | |
| | | integration | |
| oNB55 | TTTTTCGTCTCAGCCAATCC (SEQ | ϕC31 screen-pJK200#2; | 20 |
| | ID NO: 53) | Validation of plasmid | |
| | | integration | |
| oNB95 | aaaaaaaaaaaaggcgcgccTTCCGCATTTT | amplifies AscI PMA3296-8 | This |
| | GGACAGACGAAA (SEQ ID NO: 54) | fwd | study |
| oNB96 | aaaaaaaaaaaaggatccGAGATCCTTTGC | amplifies BamHI PMA3296-8 | This |
| | GCTTTTCTACGAAA (SEQ ID NO: | rev | study |
| | 55) | | |
| oNB98 | ACCTCTTACCGTGCATATGTCTTG | amplifies upstream of | This |
| | AGTTTAG (SEQ ID NO: 56) | MA3298 rev | study |
| oNB99 | AACGAAATTTTTCGTAGAAAAGCG | amplifies downstream of | This |
| | CAAAGGA (SEQ ID NO: 57) | MA3298 fwd | study |
| oNB103 | aaaaaaaaaaaacatATGTACGTGGTAAA | NdeI MA3298 fwd | This |
| | CCCGGAAGAAAAAGT (SEQ ID NO: | | study |
| | 58) | | |
| oNB104 | aaaaaaaaaaaaggatccGAGATCCTTTGC | MA3298 BamHI rev | This |
| | GCTTTTCTACGAAA (SEQ ID NO: | | study |
| | 59) | | |
| oNB121 | GCACCCAGGCACATTGTTC (SEQ | hdrA 301 rev | 5 |
| | ID NO: 60) | | |
| oNB122 | TACTGGGGTTTCTGGGAGAC (SEQ | hdrA 1024 rev | 5 |
| | ID NO: 61) | | |
| oNB123 | ATGCCCTCTCCGTAAATGAG (SEQ | hdrA 1880 fwd | 5 |
| | ID NO: 62) | | |
| oNB124 | GATTCAAGCACACTGCGATC (SEQ | hdrC 2616 rev | 5 |
| | ID NO: 63) | | |

Plasmids

| Plasmid | | |
|---|---|---|
| name | genotype | Reference |
| pJK026A | attB cat pac hpt uidA | 20 |
| pJK027A | attB cat pac hpt uidA | 20 |
| pNB710 | pJK027A:NdeI MA3298 BamHI Pₜₑₜ promoter | This |
| | | study |
| pNB711 | pJK026A:AscI MA3296-8 BamHI native promoter | This |
| | | study |
| pNB714 | pJK026A:Nde MA3298 BamHI P_{mcr} promoter | This |
| | | study |

Organisms

| | | | |
|---|---|---|---|
| NB# | Organism | genotype | Reference |

Escherichia coli strains

| | | | |
|---|---|---|---|
| 3 | DH5a F′ lac19 | F′ proA⁺B⁺ lacI^{q} Δ(lacZ) | New |
| | | M15 zzf:: Tn10 (TetR)/ | England |
| | | fhuA2Δ(argF-lacZ)U169 phoA glnV44 | Biolabs |
| | | Φ80Δ(lacZ)M15 gyrA96 recA1 endA1 | |
| | | thi-1 hsdR17 | |
| 10 | DH10B att::pAMG27 | F-mcrA Δ(mrr-hsdRMS-mcrBC) | 20 |
| | | φ80lacZΔM15 | |
| | | ΔlacX74 recA1 araD139 Δ(ara-leu) | |
| | | 7697 galU | |
| | | galK rpsL (StrR) endA1 nupGλ- | |
| | | att::Prha trfA33-254D | |
| 55 | DH10B | F-mcrA Δ(mrr-hsdRMS-mcrBC) | This |
| | att::pAMG27/pNB710 | φ80lacZΔM15 | study |
| | | ΔlacX74 recA1 araD139 Δ(ara-leu) | |
| | | 7697 galU | |
| | | galK rpsL (StrR) endA1 nupGλ- | |
| | | att::Prha trfA33-254D/pNB710 | |
| 58 | DH10B | F-mcrA Δ(mrr-hsdRMS-mcrBC) | This |
| | att::pAMG27/pNB711 | φ80lacZΔM15 | study |
| | | ΔlacX74 recA1 araD139 Δ(ara-leu) | |
| | | 7697 galU | |
| | | galK rpsL (StrR) endA1 nupGλ- | |
| | | att::Prha trfA33-254D/pNB711 | |
| 84 | DH10B | F-mcrA Δ(mrr-hsdRMS-mcrBC) | This |
| | att::pAMG27/pNB714 | φ80lacZΔM15 | study |
| | | ΔlacX74 recA1 araD139 Δ(ara-leu) | |
| | | 7697 galU | |
| | | galK rpsL (StrR) endA1 nupGλ- | |
| | | att::Prha trfA33-254D/pNB714 | |

Methanosarcina acetivorans C2A strains

| | | | |
|---|---|---|---|
| 34 | parent | Δhpt::ϕC31 int attP | 20 |
| 36 | ΔhdrABC | Δhpt::ϕC31 int attP ΔhdrA1B1C1 | 15 |
| 111 | PₜₑₜcomDE⁺ | Δhpt::ϕC31 int att:pNB710 | This |
| | | | study |
| 112 | ΔhdrABC PₜₑₜcomDE⁺ | Δhpt::ϕC31 int att:pNB710 | This |
| | | ΔhdrA1B1C1 | study |
| 114 | com⁺ | Δhpt::ϕC31 int att:pNB711 | This |
| | | | study |
| 115 | ΔhdrABC com⁺ | Δhpt::ϕC31 int att:pNB711 | This |
| | | ΔhdrA1B1C1 | study |
| 117 | PmcrcomDE+ | Δhpt::ϕC31 int att:pNB714 | This |
| | | | study |
| 118 | ΔhdrABC P_{mcr}comDE⁺ | Δhpt::ϕC31 int att:pNB714 | This |
| | | ΔhdrA1B1C1 | study |

TABLE 2
Genes overexpressed in this study.

| MA# | location | Gene ID | Predicted function |
|---|---|---|---|
| MA3296 | 4068167-4068631 | MA_RS17195 | hypothetical |
| MA3297 | 4068958-4070208 | MA_RS17200 | Cysteate synthase |
| MA3298 | 4070349-4071512 | comDE | Sulfopyruvate |
| | | | decarboxylase, |
| | | | beta |

TABLE 3
Growth rates on Methanol or Methanol + Acetate as energy sources.

| | Methanol | | | | Methanol + Acetate | | | |
|---|---|---|---|---|---|---|---|---|
| | Doubling | Std | P vs | P vs | Doubling | Std | P vs | P vs |
| Strain | time (h) | dev | parent | ΔhdrABC | time (h) | dev | parent | ΔhdrABC |
| Parent | 10.27 | 0.518 | 1 | 0.0190 | 10.81 | 0.139 | 1 | 0.0001 |
| PₜₑₜcomDE⁺ | 10.31 | 0.421 | NS | 0.0171 | 10.76 | 0.142 | NS | 0.0001 |
| P_{mcr}comDE⁺ | 8.97 | 0.233 | 0.0032 | 0.0005 | 8.22 | 0.196 | 2E−08 | 2E−08 |
| com⁺ | 10.15 | 0.590 | NS | 0.0088 | 9.68 | 0.323 | 0.0002 | 7E−06 |
| ΔhdrABC | 12.64 | 1.010 | 0.0190 | 1 | 11.79 | 0.258 | 0.0001 | 1 |
| ΔhdrABC PₜₑₜcomDE⁺ | 15.39 | 1.651 | 0.0012 | NS | 13.19 | 1.446 | 0.0145 | NS |
| ΔhdrABC P_{mcr}comDE⁺ | 15.07 | 0.738 | 0.0004 | NS | 11.95 | 0.349 | 0.0006 | NS |
| ΔhdrABC com⁺ | 15.90 | 2.924 | 0.0166 | NS | 12.84 | 1.516 | 0.0284 | NS |

NS: not significant (p > 0.05). Data were obtained from at least four biological replicates.

TABLE 4
Growth rates in medium with methanol as
sole energy source without added sulfide.

| | Doubling | Std | P vs | P vs |
|---|---|---|---|---|
| Strain | time (h) | dev | parent | ΔhdrABC |
| Parent | 9.64 | 0.464 | 1 | 0.0165 |
| P_{mcr}comDE⁺ | 8.96 | 0.231 | NS | 0.0034 |
| com⁺ | 9.37 | 0.689 | NS | 0.0224 |
| ΔhdrABC | 10.71 | 0.336 | 0.0165 | 1 |
| ΔhdrABC P_{mcr}comDE⁺ | 9.89 | 0.252 | NS | 0.0302 |
| ΔhdrABC com⁺ | 12.26 | 0.771 | 0.0128 | NS |

NS: not significant (p > 0.05). Data were obtained from at least three biological replicates. Data for parent and ΔhdrABC strains are from Salvi et al. as part of the same experiment for comparison.¹⁶

TABLE 5
Thiol quantification from MeOH-grown cells (nmol mg⁻¹). ^{a}

| | | | | |
|---|---|---|---|---|
| Strain | parent | ΔhdrABC | com⁺ | ΔhdrABC com⁺ |

Free CoM-SH

| | | | | |
|---|---|---|---|---|
| Ave | 23.24 | 15.35 | 23.69 | 19.58 |
| Std Dev | 3.70 | 4.33 | 3.82 | 3.10 |
| P vs parent | 1 | 0.000 | NS (0.787) | 0.029 |
| P vs ΔhdrABC | 0.000 | 1 | 0.000 | 0.034 |

Total CoM

| | | | | |
|---|---|---|---|---|
| Ave | 28.16 | 16.66 | 26.11 | 19.95 |
| Std Dev | 3.81 | 4.97 | 4.59 | 2.91 |
| P vs parent | 1 | 0.000 | NS (0.273) | 0.000 |
| P vs ΔhdrABC | 0.000 | 1 | 0.000 | NS (0.115) |

Ratio (% CoM-SH/Total CoM)

| | | | | |
|---|---|---|---|---|
| Ave | 82.33% | 92.56% | 91.02% | 98.37% |
| Std Dev | 0.04 | 0.05 | 0.05 | 0.09 |
| P vs parent | 1 | 0.000 | 0.000 | 0.000 |
| P vs ΔhdrABC | 0.000 | 1 | NS (0.511) | NS (0.133) |

Free CoB-SH

| | | | | |
|---|---|---|---|---|
| Ave | 3.94 | Not measurable | | Not measurable |
| Std Dev | | — | | — |
| P vs parent | 1 | — | 0.101 | — |

Total CoB

| | | | | |
|---|---|---|---|---|
| Ave | 3.94 | Not measurable | 5.79 | Not measurable |
| Std Dev | 1.25 | — | 1.87 | — |
| P vs parent | 1 | — | 0.880 | — |

Ratio (% CoB-SH/Total CoB)

| | | | | |
|---|---|---|---|---|
| Ave | 43.01 | — | 74.22 | — |
| Std Dev | 16.43 | — | 52.45 | — |
| P vs parent | 1 | — | 0.260 | — |

^{a} CoM was quantified using a standard curve with authentic mBBr-derivatized CoM. Data were obtained from at least eight biological replicates.
NS: not significant (p > 0.05).
b. CoB was identified by comparison to synthesized CoB and quantified versus the standard curve generated with authentic mBBr-derivatized CoM.⁵

### REFERENCES FOR EXAMPLE 1

1 Thauer, R. K., Kaster, A. K., Seedorf, H., Buckel, W. & Hedderich, R. Methanogenic archaea: ecologically relevant differences in energy conservation. Nat Rev Microbiol 6, 579-591, nrmicro1931 [pii]10.1038/nrmicro1931 (2008).
2 Sowers, K. R., Baron, S. F. & Ferry, J. G. Methanosarcina acetivorans sp. nov., an Acetotrophic Methane-Producing Bacterium Isolated from Marine Sediments. Appl Environ Microbiol 47, 971-978 (1984).
3 Buan, N. R. Methanogens: pushing the boundaries of biology. Emerging Topics in Life Sciences 2, 629-646, 10.1042/etls20180031 (2018).
4 De Vrieze, J., Hennebel, T., Boon, N. & Verstraete, W. Methanosarcina: the rediscovered methanogen for heavy duty biomethanation. Bioresour Technol 112, 1-9, 10.1016/j.biortech.2012.02.079 (2012).
5 Catlett, J. L., Ortiz, A. M. & Buan, N. R. Rerouting Cellular Electron Flux To Increase the Rate of Biological Methane Production. Appl Environ Microbiol 81, 6528-6537, 10.1128/AEM.01162-15 AEM.01162-15 [pii](2015).
6 Duszenko, N. & Buan, N. R. Physiological Evidence for Isopotential Tunneling in the Electron Transport Chain of Methane-Producing Archaea. Appl Environ Microb 83, UNSP e00950 10.1128/AEM.00950-17 (2017).
7 Aldridge, J., Carr, S., Weber, K. A. & Buan, N. R. Anaerobic Production of Isoprene by Engineered Methanosarcina Species Archaea. Appl Environ Microbiol 87, 10.1128/AEM.02417-20 (2021).
8 Schöne, C., Poehlein, A., Jehmlich, N., Adlung, N., Daniel, R., von Bergen, M., Scheller, S. & Rother, M. Deconstructing Methanosarcina acetivorans into an acetogenic archaeon. Proc Natl Acad Sci USA 119, 10.1073/pnas.2113853119 (2022).
9 Soo, V. W., McAnulty, M. J., Tripathi, A., Zhu, F., Zhang, L., Hatzakis, E., Smith, P. B., Agrawal, S., Nazem-Bokaee, H., Gopalakrishnan, S., Salis, H. M., Ferry, J. G., Maranas, C. D., Patterson, A. D. & Wood, T. K. Reversing methanogenesis to capture methane for liquid biofuel precursors. Microb Cell Fact 15, 11, 10.1186/s12934-015-0397-z 10.1186/s12934-015-0397-z [pii](2016).
10 McAnulty, M. J., Poosarla, V. G., Li, J., Soo, V. W., Zhu, F. & Wood, T. K. Metabolic engineering of Methanosarcina acetivorans for lactate production from methane. Biotechnol Bioeng 114, 852-861, 10.1002/bit.26208 (2017).
11 Holmes, D. E., Zhou, J., Ueki, T., Woodard, T. & Lovley, D. R. Mechanisms for Electron Uptake by Methanosarcina acetivorans during Direct Interspecies Electron Transfer. mBio 12, e0234421, 10.1128/mBio.02344-21 (2021).
12 McBride, B. C. & Wolfe, R. S. A new coenzyme of methyl transfer, coenzyme M. Biochemistry 10, 2317-2324, 10.1021/bi00788a022 (1971).
13 Noll, K. M., Rinehart, K. L., Jr., Tanner, R. S. & Wolfe, R. S. Structure of component B (7-mercaptoheptanoylthreonine phosphate) of the methylcoenzyme M methylreductase system of Methanobacterium thermoautotrophicum. Proc Natl Acad Sci USA 83, 4238-4242, 10.1073/pnas.83.12.4238 (1986).
14 Graham, David E., Taylor, Stephanie M., Wolf, Rachel Z. & Namboori, Seema C. Convergent evolution of coenzyme M biosynthesis in the Methanosarcinales: cysteate synthase evolved from an ancestral threonine synthase. Biochemical Journal 424, 467-478, 10.1042/bj20090999 (2009).
15 Buan, N. R. & Metcalf, W. W. Methanogenesis by Methanosarcina acetivorans involves two structurally and functionally distinct classes of heterodisulfide reductase. Mol Microbiol 75, 843-853, MMI6990 [pii]10.1111/j.1365-2958.2009.06990.x (2010).
16 Salvi, A. M., Chowdhury, N. B., Saha, R. & Buan, N. R. Supplementation of Sulfide or Acetate and 2-Mercaptoethane Sulfonate Restores Growth of the Methanosarcina acetivorans DeltahdrABC Deletion Mutant during Methylotrophic Methanogenesis. Microorganisms 11, 10.3390/microorganisms11020327 (2023).
17 Sowers, K. R., Boone, J. E. & Gunsalus, R. P. Disaggregation of Methanosarcina spp. and Growth as Single Cells at Elevated Osmolarity. Appl Environ Microbiol 59, 3832-3839 (1993).
18 Uetake, H., Luria, S. E. & Burrous, J. W. Conversion of somatic antigens in Salmonella by phage infection leading to lysis or lysogeny. Virology 5, 68-91, 0042-6822(58)90006-0 [pii](1958).
19 Li, M. Z. & Elledge, S. J. Harnessing homologous recombination in vitro to generate recombinant DNA via SLIC. Nat Methods 4, 251-256, nmeth1010 [pii]10.1038/nmeth1010 (2007).
20 Guss, A. M., Rother, M., Zhang, J. K., Kulkarni, G. & Metcalf, W. W. New methods for tightly regulated gene expression and highly efficient chromosomal integration of cloned genes for Methanosarcina species. Archaea 2, 193-203 (2008).
21 Horne, A. J. & Lessner, D. J. Assessment of the oxidant tolerance of Methanosarcina acetivorans. FEMS Microbiol Lett 343, 13-19, 10.1111/1574-6968.12115 (2013).
22 Jennings, M. E., Schaff, C. W., Horne, A. J., Lessner, F. H. & Lessner, D. J. Expression of a bacterial catalase in a strictly anaerobic methanogen significantly increases tolerance to hydrogen peroxide but not oxygen. Microbiology 160, 270-278, 10.1099/mic.0.070763-0 mic.0.070763-0 [pii](2014).
23 Sneller, F. E., van Heerwaarden, L. M., Koevoets, P. L., Vooijs, R., Schat, H. & Verkleij, J. A. Derivatization of phytochelatins from Silene vulgaris, induced upon exposure to arsenate and cadmium: comparison of derivatization with Ellman's reagent and monobromobimane. J Agric Food Chem 48, 4014-4019, 10.1021/jf9903105 (2000).
24 Rijstenbil, J. W. & Wijnholds, J. A. HPLC analysis of nonprotein thiols in planktonic diatoms: pool size, redox state and response to copper and cadmium exposure. Marine Biology 127, 45-54, 10.1007/BF00993642 (1996).
25 Minocha, R., Thangavel, P., Dhankher, O. P. & Long, S. Separation and quantification of monothiols and phytochelatins from a wide variety of cell cultures and tissues of trees and other plants using high performance liquid chromatography. J Chromatogr A 1207, 72-83, 10.1016/j.chroma.2008.08.023 (2008).
26 Duin, E. C., Prakash, D. & Brungess, C. Methyl-coenzyme M reductase from Methanothermobacter marburgensis. Methods Enzymol 494, 159-187, 10.1016/B978-0-12-385112-3.00009-3 (2011).
27 Welte, C. & Deppenmeier, U. Proton translocation in methanogens. Methods Enzymol 494, 257-280, 10.1016/B978-0-12-385112-3.00013-5 (2011).
28 Fahey, R. C., Brown, W. C., Adams, W. B. & Worsham, M. B. Occurrence of glutathione in bacteria. Journal of Bacteriology 133, 1126-1129, doi:10.1128/jb.133.3.1126-1129.1978 (1978).
29 Kistler, M., Maier, K. & Eckardt-Schupp, F. Genetic and biochemical analysis of glutathione-deficient mutants of Saccharomyces cerevisiae. Mutagenesis 5, 39-44, 10.1093/mutage/5.1.39 (1990).
30 Copley, S. D. & Dhillon, J. K. Lateral gene transfer and parallel evolution in the history of glutathione biosynthesis genes. Genome Biol 3, research0025 (2002).
31 Jasso-Chavez, R., Santiago-Martinez, M. G., Lira-Silva, E., Pineda, E., Zepeda-Rodriguez, A., Belmont-Diaz, J., Encalada, R., Saavedra, E. & Moreno-Sanchez, R. Air-adapted Methanosarcina acetivorans shows high methane production and develops resistance against oxygen stress. PLoS One 10, e0117331, 10.1371/journal.pone.0117331 PONE-D-14-41605 [pii](2015).
32 Lira-Silva, E., Santiago-Martinez, M. G., Garcia-Contreras, R., Zepeda-Rodriguez, A., Marin-Hernandez, A., Moreno-Sanchez, R. & Jasso-Chavez, R. Cd2+ resistance mechanisms in Methanosarcina acetivorans involve the increase in the coenzyme M content and induction of biofilm synthesis. Environ Microbiol Rep 5, 799-808, 10.1111/1758-2229.12080 (2013).
33 Durichen, H., Diekert, G. & Studenik, S. Redox potential changes during ATP-dependent corrinoid reduction determined by redox titrations with europium(II)-DTPA. Protein Sci 28, 1902-1908, 10.1002/pro.3699 (2019).
34 Huening, K. A., Jiang, R. & Krzycki, J. A. Kinetic and substrate complex characterization of RamA, a corrinoid protein reductive activase from Methanosarcina barkeri. FEMS Microbiol Lett 367, 10.1093/femsle/fnaa128 (2020).
35 Lu, Z. & Imlay, J. A. When anaerobes encounter oxygen: mechanisms of oxygen toxicity, tolerance and defence. Nat Rev Microbiol 19, 774-785, 10.1038/s41579-021-00583-y (2021).
36 Fridovich, I. Superoxide radical and superoxide dismutases. Annu Rev Biochem 64, 97-112, 10.1146/annurev.bi.64.070195.000525 (1995).
37 Santos, L. O., Silva, P. G. P., Lemos Junior, W. J. F., de Oliveira, V. S. & Anschau, A. Glutathione production by Saccharomyces cerevisiae: current state and perspectives. Appl Microbiol Biot 106, 1879-1894, 10.1007/s00253-022-11826-0 (2022).
38 McElroy, C. & Jennewein, S. in Biotechnology of Natural Products (eds Wilfried Schwab, Bernd Markus Lange, & Matthias Wust) 145-185 (Springer International Publishing, 2018).
39 Carr, S. & Buan, N. R. Insights into the biotechnology potential of Methanosarcina. Front Microbiol 13, 1034674, 10.3389/fmicb.2022.1034674 (2022).

### Example 2—In Vivo Synthesis of the Methanogenic Cofactor Coenzyme M (MESNA) Protects Escherichia coli from Oxidative Stress

Oxidative stress is a ubiquitous challenge for all organisms regardless of whether they grow aerobically or anaerobically.(1, 2) To combat this challenge, organisms synthesize low-molecular weight thiols such as glutathione to scavenge oxidants before they can damage proteins, lipids, or nucleic acids. Coenzyme M (CoM) is a low-molecular weight thiol used as a methyl carrier by anaerobic methane-producing archaea (methanogens) which we proposed could have a potential secondary role as an antioxidant. While many of the initial steps for the synthesis of CoM in methanogens are well characterized, the critical last step has remained enigmatic.(3) We examined the genome of Methanosarcina acetivorans and hypothesized that the final enzyme in the CoM biosynthetic pathway, comF, was encoded by the gene facing tail-to-tail with comDE. To assess whether comF is responsible for the last step in CoM biosynthesis, we designed a synthetic operon (com^{syn}) which co-expressed comF with taurine-pyruvate aminotransferase (tpa). The expression of com^{syn} in Escherichia coli resulted in the synthesis of CoM, confirmed using reverse phase high-performance liquid chromatography. Furthermore, we observed CoM acted as an antioxidant and hypothesized that com^{syn} could be used to improve resistance to oxidative stress in E. coli. Physiological studies exposing com^{syn} E. coli to reactive oxygen species confirmed a greater resistance to oxidative stress. Our findings suggest ComF can catalyze the final step of CoM biosynthesis and that CoM can mitigate oxidative stress in non-methanogenic organisms, even when grown aerobically.

### Keywords

Coenzyme M, CoM, Mesna, Antioxidant, Redox, Oxidative Stress, ROS, Methanogen, archaea.

### Significance Statement

Coenzyme M (C₂H₅O₃S₂) is the smallest organic coenzyme and serves as a one-carbon carrier in methane biosynthesis and anaerobic alkane oxidation. Identifying comF has implications for metagenomic predictions of CoM-dependent metabolic processes in microbes responsible for methanogenesis, methane oxidation, and subsurface alkane metabolism. CoM is an important chemotherapeutic adjuvant used as an anti-toxicity renal protectant. Our findings raise the possibility for introducing CoM biosynthetic pathways into a non-native organism where it has the potential to complement glutathione and other endogenous redox homeostasis systems. Predicted uses include increasing photosynthetic efficiency in plant crops, aquaculture, and in both microbial fermentation and biomanufacturing, serving as a redox buffer to react with organometallic tetrapyrrole cofactors (B₁₂, heme, F₄₃₀) or to enable synthesis of new compounds.

### Introduction

Antioxidants are molecules that accept or donate electrons to reactive oxidative species (ROS) thereby preventing damage to DNA, RNA, lipids, proteins, and other metabolites.(4) Oxidative stress occurs when ROS accumulates and organisms produce ROS-scavenging antioxidants to prevent cell damage. For example, increased concentrations of antioxidant compounds have been found to prevent death in Chinese hamster ovary cells, the cell line responsible for the production of 70% of therapeutic proteins.(5, 6) Antioxidant systems to combat ROS predate the rise of molecular oxygen in the atmosphere indicating that defense against ROS was important for survival on early Earth, possibly as a defense against geologically-sourced H₂O₂ or H₂O₂ generated from UV-induced photolysis of water.(7-9) The development of an oxidizing environment due to emergence of oxidative photosynthesis enhanced the importance of antioxidant systems for maintaining redox homeostasis and protecting vulnerable cofactors.(10) Low molecular weight (LMW) thiols are small molecules possessing a reactive sulfhydryl (—SH) functional group that can function as antioxidants to maintain redox homeostasis.(11) LMW thiols also serve as a form of storage for cysteine, form complexes with metal ions to prevent toxicity, and detoxify both xenobiotics and endogenous electrophiles.(12-15) The most prevalent LMW thiols in cells are generally considered to be glutathione (GSH) and cysteine, however many prokaryotes do not synthesize GSH and there are numerous alternative LMW thiols used by diverse organisms for a variety of purposes.(16, 17)

Methanogens are considered strict obligate anaerobes, however almost all obligative and facultative anaerobes have defenses against oxidative stress in the form of SODs, catalases, and peroxidases. Compared to other methanoarchaeal families, to date Methanosarcinales spp. possess the most extensive set of genes annotated to encode proteins implicated in oxidative stress protection including multiple peroxidases, uncommon in other methanogen genera. CoM, pharmacologically known as Mesna, is known to act as an antioxidant however CoM biosynthesis was only recently implicated in protecting Methanosarcina acetivorans from oxidative stress.(18, 19) In previous work, we observed that ΔhdrABC deletion mutants, which upregulate predicted CoM biosynthetic genes, were highly resistant to O₂ and H₂O₂ stresses.(20, 21) When M. acetivorans was adapted to grow under conditions of 0.4-1% oxygen over the course of 6 months, transcriptional and enzymatic activities of SOD, CAT, and PXs were significantly increased, coinciding with 2 times higher intracellular concentrations of cysteine, CoM, and sulfide.(22) These lines of evidence directly implicate CoM in protecting the strictly anaerobic M. acetivorans from ROS, in addition to playing a role as C1 carrier in methanogenesis.

Based on the chemical properties of CoM and the widespread use of LMW thiols for redox homeostasis in biology, we hypothesized that CoM could be used as an antioxidant by organisms that do not endogenously produce CoM. To test this hypothesis, we would need to introduce the genes responsible for the production of CoM into a heterologous host organism such as E. coli. Methanogens have been discovered to synthesize CoM by one of two canonical routes that merge after the formation of 3-sulfopyruvate (FIG. 1). In Class I methanogens CoM is derived from phosphoenolpyruvate and is converted to 3-sulfopyruvate via the enzymes ComA, ComB, and ComC.(23-25) M. acetivorans lacks these enzymes; instead the organism utilizes an alternative pathway to generate 3-sulfopyruvate using aspartate aminotransferase and phosphoserine aminotransferase to convert L-cysteate to 3-sulfopyruvate.(26) The L-cysteate is metabolized from O-phospho-L-serine using a cysteate synthase, MA3297.(27) After the point of metabolic convergence, methanogens utilize ComDE to generate sulfoacetaldehyde thought to be metabolized by an unknown “ComF” enzyme to generate CoM.(3) Unfortunately, the enzyme(s) having ComF function remains elusive. Thus, confident assignments for CoM biosynthetic enzymes are not always obvious and the possibility of diverse pathways for CoM synthesis has been a challenging puzzle to unravel. In this work, we describe identification of ComF activity in a gene derived from M. acetivorans, demonstrate in vivo CoM production in E. coli using a synthetic pathway, and show that this in vivo production of CoM confers durable protection from ROS.

### Materials and Methods

Strains and growth conditions. Strains are listed in Table 1. E. coli DH5a (NB3) was used for plasmid construction, E. coli BL21 (NB8) was used in the enzymatic assays, and E. coli K-12 was used in the oxidative stress tests (Table 6). E. coli K-12 wild-type and ΔtauD from the Keio collection were purchased through the Coli Genetic Stock Center (NB398 and NB494, respectively) (New Haven, CT). The kanamycin resistance cassette in the ΔtauD strain was removed using pCP20 expressing FLP recombinase to create NB499.(28) To express T7 polymerase, strains were lysogenized using the λDE3 Lysogenization Kit from Novagen® a brand of EMD Biosciences Inc. (San Diego, CA). Strains were grown in either Luria Broth (LB) or defined media (Table 7).(29-33) Media were supplemented with cumene (2.4 μM), cumene hydroperoxide (CuHO₂) (2.4 μM), kanamycin (50 μg/mL), ampicillin (100 μg/mL), isopropyl ß-D-1-thiogalactopyranoside (IPTG), L-arabinose (2%), cysteine HCl (2 mM), ferric ammonium citrate (1 mM), and iron (II) sulfate (1 mM) where indicated. All chemicals were obtained from Millipore Sigma (Burlington, MA) or ThermoFisher (Waltham, MA).

Uptake of exogenous CoM by E. coli. To determine whether E. coli could catabolize CoM, wild-type (NB506) and ΔtauD (NB510) E. coli K-12 strains were tested for the ability to use CoM, cysteine, and taurine as sole sulfur source on M9 minimal medium lacking sulfur (—S) agar plates after washing to reduce nutrient carryover. E. coli strains were streaked on LB agar (1.5%) plates and used to inoculate 3 mL defined medium (Table 7) then incubated at 35° C. (16 hours, 215 rpm). Cells from 100 μL of each culture was harvested by centrifugation at 5,000×g for 3 minutes. Cells were washed three times and resuspended in 1 mL phosphate buffered saline (137 mM NaCl, 2.7 mM KCl, 10 mM Na₂HPO₄, 1.8 mM KH₂PO₄). M9 (—S) minimal media agar plates were prepared (23.9 mM Na₂HPO₄, 11 mM KH₂PO₄, 4.3 mM NaCl, 9.35 mM NH₄Cl, 2 mM MgCl₂, 0.1 mM CaCl₂, 0.4% glucose, and 1.5% agar) and cysteine, taurine, α-ketoglutaric acid, and coenzyme M solutions (100 μM) were spread on the M9 (—S) plates along with a negative H₂O control then allowed to dry in an incubator for 30 minutes. Washed cultures were streaked on each agar plate and incubated at 35° C. for 63 hours before photographing.

For oxidative stress assays, 3 mL defined medium was inoculated (1:100) from overnight cultures then supplemented with or without 100 μM CoM at 37° C. When OD600=0.1, each culture was diluted to OD600=0.01 with fresh defined medium without CoM (maximum final concentration after dilution was 10 μM), then 148 μL was dispensed to sterile 96-well plates supplemented with 2 μL of either cumene (75 μM final concentration) or CuHO₂ (45-75 μM final concentration) as indicated. Culture turbidity at 600 nm was measured in a Tecan Sunrise plate reader (Männedorf, Switzerland) at 37° C. over 18 hours. Lag time was calculated as the time taken to reach OD600=0.1.

Gene parts and plasmid construction. Plasmids, synthesized gene/constructs, and oligonucleotide primers are listed in Table 6. The amino acid sequences of MA3299 (ComF, Q8TKU5) and taurine-pyruvate aminotransferase (Tpa, Q9APM5) were derived from the genomes of M. acetivorans and Bilophila wadsworthia respectively. Each gene was codon optimized for expression in E. coli (IDT, Coralville, IA) and restriction enzyme sites were removed. Prior to the start codon of the first gene, tpa, a Shine-Dalgarno Sequence (AGGAGG) and NdeI restriction enzyme site were added. To create the com^{syn} synthetic operon, another Shine-Dalgarno Sequence (AGGAGG) and a BamHI restriction enzyme site were added between the two genes. A 4×Gly-6×His tag was added to the amino terminus of each protein and a SacI restriction enzyme site was added after the second gene, comF. Gene strings sNB25, containing comF, and sNB34, containing tpa, were obtained from ThermoFisher (Waltham, MA) and oligonucleotide primers were obtained from IDT (Coralville, IA). sNB25 and sNB34 were ligated together after restriction enzyme digest and subsequently amplified as a single piece of DNA by PCR. The final com^{syn} construct was inserted into a pET24a expression vector using NEBuilder HiFi Assembly (New England Biolabs, Ipswich, MA) and transformed into DH5a E. coli. The resulting plasmid pCH003 was sequenced by Eurofins (Louisville, KY).

Confirmation of protein expression. pCH003 encoding the synthetic com^{syn} operon was transformed into BL21 E. coli for protein expression. Cultures were grown in LB medium at 37° C. (250 rpm), induced at OD600=0.5-0.7 using various concentrations of IPTG and grown for three more hours at 37° C. Cultures (1.5 mL) were pelleted at 5,000 rpm for five minutes, lysed in 2×Laemmli Buffer at 100° C. for 10 minutes, centrifuged at 21,100×g for 20 minutes, and 10 μL of each culture was added to the wells of a 12.5% SDS-polyacrylamide gel.(34) Proteins were separated with a constant 100V (45 minutes). For LC-MS/MS analysis, bands were visualized using Coomassie Stain and Destain; the bands centering at 50 kDa were submitted to the UNL Proteomics Core for analysis.(35) For immunoblot analysis, a BenchMark™ His-tagged Protein Standard from Invitrogen (Waltham, MA) was used and all proteins were transferred from the SDS-polyacrylamide gel to a polyvinylidene difluoride (PVDF) membrane using constant 14 V for 16 hours in a 15% methanol transfer buffer. The membrane was dried, blocked using 10% milk in Tris-buffered saline (TBS) for 10 minutes with constant stirring, rinsed using TBS, and incubated 6×-His Tag Monoclonal Antibody (3D5), horseradish peroxidase (HRP) from (Waltham, MA) at a concentration of 1.5:5000 in TBS with 5% milk with constant stirring for 1 hour. The membrane was then incubated with SuperSignal™ West Pico PLUS Chemiluminescent Substrate (Thermo Fisher Scientific, Waltham, MA) and imaged using a LI-COR Odyssey Fc (Lincoln, NE).

CoM biosynthesis assay. To improve expression of comF, pCH003 was transformed into BL21 E. coli along with pDB1282, graciously provided by Squire J. Booker (Penn State University).(36) This strain was grown in 25 mL defined medium overnight at 37° C. (250 rpm); this was used to inoculate 975 mL defined medium and continued growth at 37° C. (250 rpm). At OD600=0.3 of the culture was supplemented with 2% L-arabinose, 2 mL 1 M cysteine HCl (2 mM), 0.5 M iron (II) sulfate (1 mM), and 0.5 M ferric ammonium sulfate (1 mM) then returned to incubation. After 1 hour the culture was induced using 0.1 mM IPTG, incubated for 3 hours, pelleted at 8,000×g (10 minutes, 4° C.), and stored at −80° C. until analysis. A portion of the cell pellet was lysed as described above and the presence of ComF and Tpa was confirmed by immunoblotting using a 8-16% polyacrylamide gel with the initial electrophoresis changed to 50 V (25 minutes) followed by 100 V (105 minutes) and transferred using constant 0.11 A (50 minutes). For enzyme assays, 1.56 g of cell pellet was brought into the anaerobic chamber then resuspended in 10 mL anaerobic lysis buffer (25 mM Tris-HCl (pH 8), 25 mM KCl, and 2% glycerol in H₂O). 2 mL lysis buffer was reserved as 1 mL aliquots in 1.5 mL glass autosampler vials. Resuspended cells were kept on ice while sonicated at 40% amplitude with 30 second on/off cycles for a total duration of 10 minutes. Three 1 mL aliquots of the resulting crude lysate were removed and kept on ice while the remainder was transferred to a 50 mL Oakridge bottle and removed from the anaerobic chamber. Cell debris was pelleted at 10,000×g (10 minutes, 4° C.) then brought back into the anaerobic chamber. Two 1 mL aliquots of soluble extract were removed while the remaining extract was passed through a 5 mL luer lock syringe fitted with a 13 mm 0.2 μm surfactant-free cellulose acetate with prefilter (SFCA/PF) filter (Corning, Corning, NY) to create two 1 mL aliquots. The insoluble fraction was resuspended in lysis buffer then 1 mL was aliquoted into two vials. All aliquots were each given 20 μL 0.5M iron (II) sulfate heptahydrate (10 mM) and 20 μL 0.5 M sodium sulfide nonahydrate (10 mM) then mixed by inversion and incubated for 20 minutes at room temperature. Two aliquots of each were given 20 μL 0.5 M sodium pyruvate (final: 10 mM), 20 μL 0.5 M taurine (final: 10 mM), and 20 μL 2.5 M potassium borohydride (final: 50 mM). The third aliquot of crude lysate was given 10 μL 0.1 M CoM (0.4 mM) and all aliquots were inverted to mix then incubated at 37° C. 250 μL samples were taken into 1.5 mL glass autosampler vials at 1 minute, 10 minutes, 30 minutes, and 5 hours. Samples were immediately removed from the anaerobic chamber and diluted with 635 μL 200 mM 4-(2-hydroxyethyl)-1-piperazineethanesulfonic acid (HEPES) and 5 mM pentetic acid (DTPA) (pH 8.2), reduced using 5 μL 80 mM TCEP (800 μM) then vortexed, incubated at 45° C. for 10 min, derivatized using 10 μL 80 mM monobromobimane (mBBr) in acetonitrile then vortexed, incubated at 45° C. for 30 minutes, and the derivatization reaction was terminated with 100 μL of 1 M methanesulfonic acid. Samples were aspirated into a luer slip tip syringe and filtered through a 0.2 μm, 13 mm diameter polyethersulfone (PES) membrane into fresh amber autosampler vials then stored at 4° C. in the dark until analyzed by high-performance liquid chromatography (HPLC) using an UltiMate 3000 (Thermo Scientific, Waltham, MA). HPLC solutions were prepared in autoclaved wide mouth bottles using HPLC grade solvents and degassed using a water bath. The reverse phase-HPLC (RP-HPLC) procedure was carried out in accordance with the method established by Minocha et al. using a Supelcosil LC-18 column (5 μm particle size, 15 cm×4.6 mm) and a LC-18 Supelguard Cartridge (5 μm particle size, 2 cm×4 mm) (Supelco, Bellefonte, PA).(37)

Acute oxidative stress assay. E. coli K-12 ΔtauD (NB510) was transformed using pET24a (vector-only control, VOC) or pCH003 (com^{syn}) to generate strains NB515 and NB525, respectively, and grown on LB agar (1.5%) plates containing 50 μg/mL kanamycin. Cultures were grown in defined medium with kanamycin at 37° C. (10 hours, 215 rpm) then 6 mL defined medium was inoculated (1:100). This culture was incubated until OD600=0.4-0.5 then induced using 0.1 mM IPTG. After another hour of incubation, the cultures were then split into two aliquots of 2.6 mL each, given either 0.1 mM taurine or H₂O, and incubated another hour. LB plates containing 0.1 mM IPTG and 0.1 mM taurine were prepared and warmed for 15 minutes at 37° C. Cultures were normalized to 0.1 OD600 and 1 mL of culture was spread on each plate. The plates were dried in the incubator for 10 minutes then 4 sterile absorption disks were placed in the center of each quadrant of the plate. One disk was given 5 μL H₂O while the other three were given 5 μL 2.45 M H₂O₂ then incubated at 37° C. (16 hours) before being imaged in a BioRad imager (Hercules, CA). Zones of clearing were measured using the ImageJ program.(38)

Chronic oxidative stress assay. Cultures were prepared according to the procedure outlined in the Acute Stress Assay method above. Cultures were normalized to 0.1 OD600 and 4.5 mL defined medium was inoculated (1:10) and gently mixed. Cultures (148 μL) were dispensed into each well of a sterile 96-well plate. 2 μL of either H₂O or 180 M CuHO₂ was added to designated wells. Culture turbidity at 600 nm was measured in a Tecan Sunrise plate reader (Männedorf, Switzerland) at 37° C. over 24 hours. Lag time was calculated as the time taken to reach OD600=0.1.

### Results

Exogenous CoM Protects E. coli from Oxidative Stress

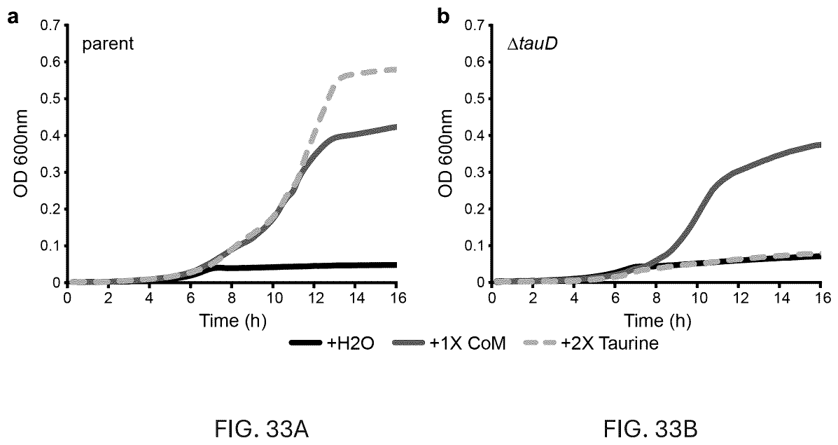

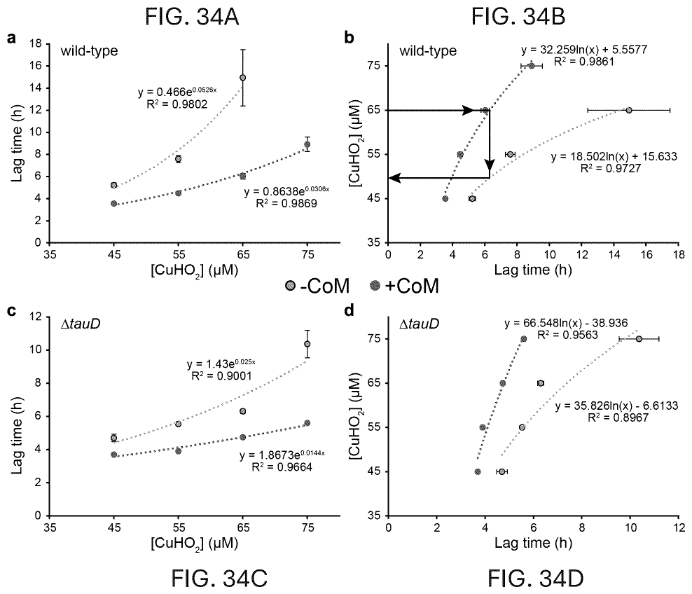

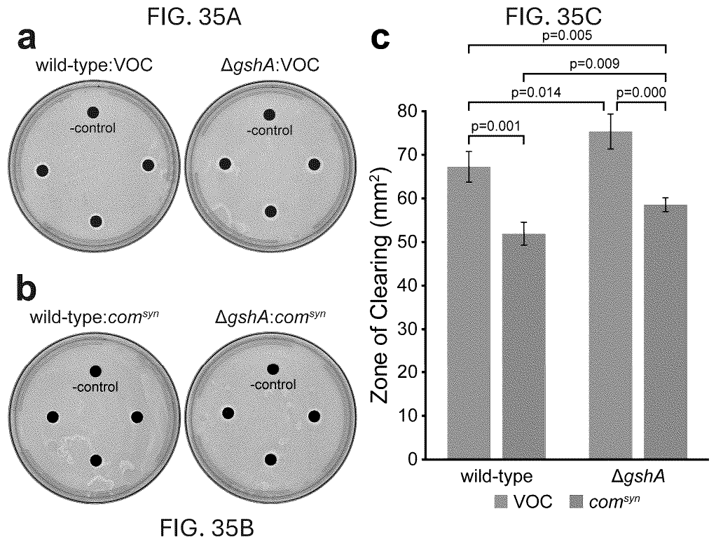

We assessed which catabolic pathways could be involved in a hypothetical digestion of CoM by E. coli. Initially characterized by Eichhorn et al., there are two systems for the utilization of alkanesulfonates as sulfur sources in E. coli, ssuEADCB and tauABCD which are expressed under the conditions of sulfate starvation.(39, 40) TauD is an α-ketoglutarate-dependent taurine dioxygenase which uses an Fe²⁺ cofactor to metabolize a range of sulfonates including, notably, taurine.(41) SsuD is a alkanesulfonate monooxygenase which uses reduced flavin mononucleotide to metabolize a comparably wider range of sulfonate containing molecules.(42) Only recently was it reported that E. coli ΔtauD could digest taurine in a ssuD-dependent manner after it had been initially reported that SsuD lacked this capability.(42, 43) The mechanism by which this catabolism occurs is still under investigation however, for our purposes, it was decided that ΔtauD would be the more interesting mutant to examine as CoM (C₂H₅O₃S₂) is structurally similar to the non-proteinogenic amino acid taurine (C₂H₇NO₃S) in that taurine has an amino group (—NH₃⁺) where CoM has a —SH group. If TauD could also catabolize CoM, we might expect the ΔtauD strain either lack the ability to or demonstrate difficulty in using CoM as a sulfur source. If this was the case, then the antioxidant effect of CoM may be stronger in the ΔtauD strain when compared to the wild-type strain. After both wild-type and E. coli K-12 ΔtauD were streaked on M9 minimal media with various sulfur sources both strains grew well on the plates supplemented with 100 μM Coenzyme M with little observable difference between them suggesting that CoM is metabolized, at least in part, through a tau-independent pathway (FIG. 33). Inhibitory growth for both strains was observed when plated with 2 mM Tau while no growth defects were observed when plated with 2 mM Cys or CoM. Furthermore, we noted that while both E. coli K12 strains grew poorly on taurine, the ΔtauD strain had a more severe growth defect that appeared similar to the no S negative control.

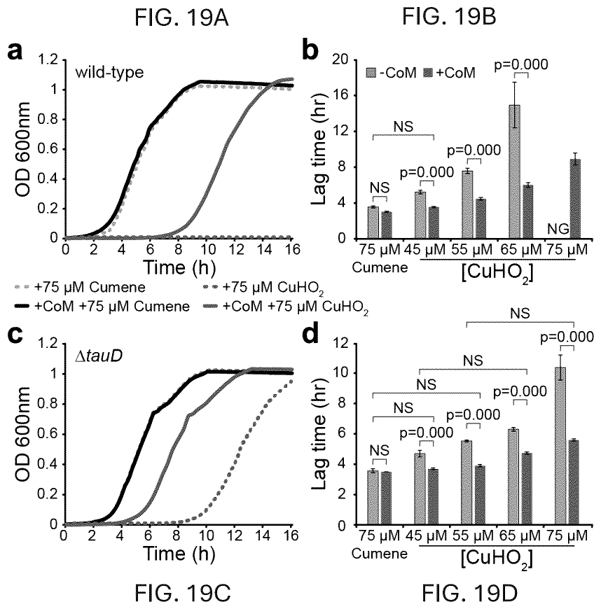

After having established that E. coli can use CoM as a sulfur source when grown on sulfur-free media, we tested if exogenously supplied CoM could mitigate oxidative stress. Cumene hydroperoxide (CuHO₂) was chosen as oxidant because it is an unstable alkyl hydroperoxide that slowly degrades to release peroxide radicals over time, in contrast to hydrogen peroxide which rapidly generates peroxide radicals into the culture medium.(44) The negative control, wild-type E. coli cells incubated with 75 μM cumene, had a lag time of 3.6 h (±0.12) without CoM (−CoM), and 3.0 h (±0.06) when preincubated with CoM (+CoM) (p=5×10⁻⁵) (FIG. 19a). When wild-type E. coli (−CoM) was subjected to 65 μM CuHO₂ the lag time was 14.9 h (±2.55), while +CoM cells subjected to 65 μM CuHO₂ had a lag time of 6.0 h (±0.26, p=5×10⁻⁴). Protection from oxidative stress remained consistent as the concentration of CuHO₂ increased (FIG. 19b). Exponential and logarithmic regression analyses were applied to the lag times of −CoM and +CoM cultures under CuHO₂ stress (FIG. 33). These regression curves and their associated equations calculate that the lag time for +CoM E. coli subjected to 65 μM CuHO₂ is equivalent to the lag time of −CoM E. coli subjected to ˜50 μM CuHO₂. This indicates that ˜15 μM CuHO₂ was mitigated by ˜30 μM CoM, assuming the stoichiometry of ROS mitigation is 1:2 of CuHO₂ to CoM. The level of protection observed was surprising as at the most only ˜8.25 μM CoM would have carried over from the starter cultures, suggesting that CoM was imported into cells and potentially was complementing the endogenous glutathione-based redox homeostasis system.

This oxidative stress assay was repeated using the ΔtauD deletion mutant (FIG. 19c). Similar to the wild-type, ΔtauD incubated with 75 μM cumene had a lag time of 3.6 h (±0.13) in the absence of CoM and 3.5 h (±0.00) when preincubated with CoM. When the ΔtauD strain (−CoM) was subjected to 65 μM CuHO₂ the lag time was 6.3 h (±0.13), while cells grown in the presence of CoM (+CoM) and subjected to 65 μM CuHO₂ had a lag time of 4.7 h (±0.08, p=2×10⁻⁷) (FIG. 19d). This lag time can be compared directly to the lag time of the −CoM culture dosed with 45 μM CuHO₂ which was 4.7 h (±0.22, p=0.788) where there is no significant difference. This pattern holds true when comparing +CoM 75 μM CuHO₂ to −CoM 55 μM CuHO₂ (p=0.243) implying ˜20 μM CuHO₂ was mitigated by ˜40 μM CoM. This data indicates that preincubation with CoM resulted in protection from CuHO₂ stress in both the wild-type and ΔtauD strains but was more pronounced in the ΔtauD strain. Comparing these two strains directly using the data provided is not advised as the experimental data for FIGS. 2a-b and FIGS. 2c-d were obtained through separate 96-well plate assays, performed on separate days, using separately prepared aliquots of CuHO₂.

Design of a Synthetic Com^{syn} Operon for CoM Production in E. coli

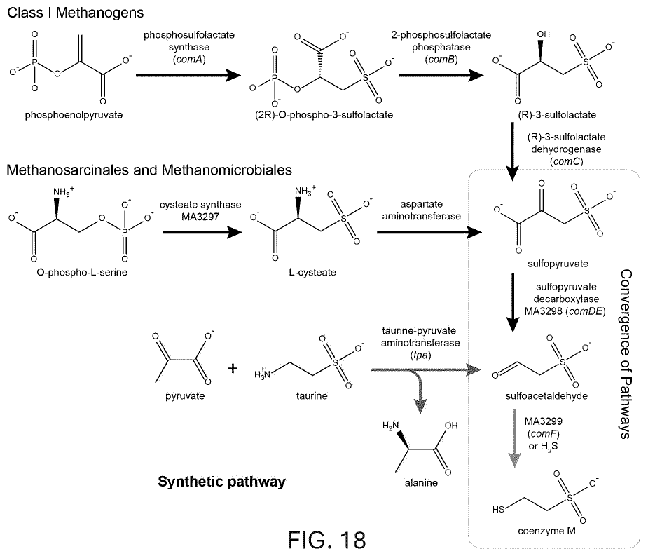

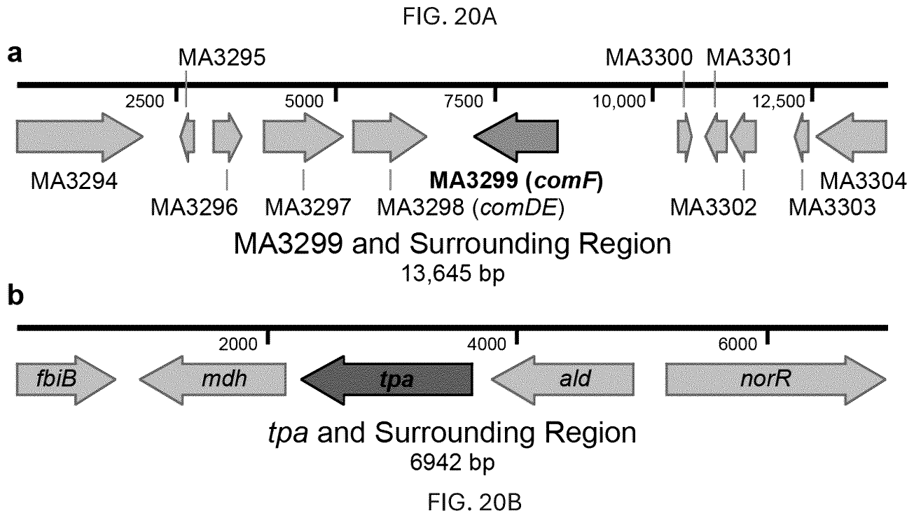

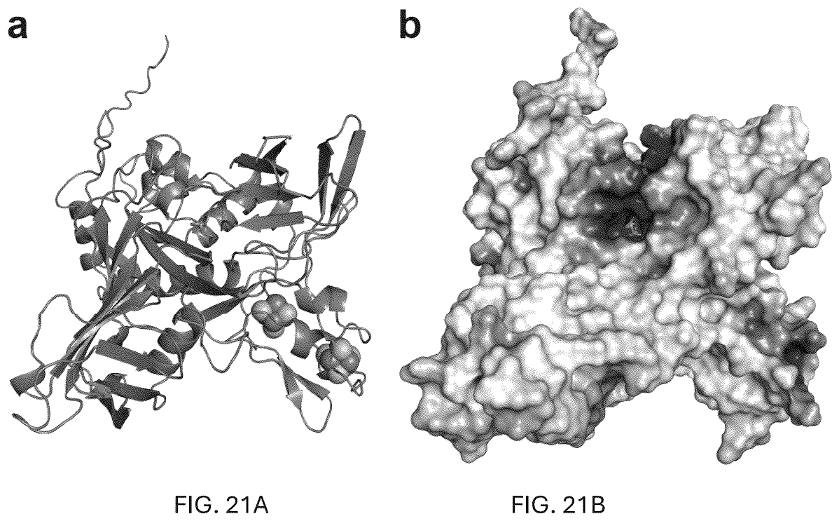

We next wanted to test whether endogenous production of CoM could also protect cells from oxidative stress. Because the central metabolisms of methanogens and E. coli are significantly different we used a reverse engineering design approach to hypothesize a synthetic biochemical pathway that could function in E. coli.(45, 46) The final known step in the biosynthesis of CoM is catalyzed by ComDE which produces sulfoacetaldehyde (SAA) via PLP-dependent decarboxylation of 3-sulfopyruvate. Examination of the M. acetivorans genome surrounding the comDE gene (FIG. 20a) suggested that MA3299, which faces tail-to-tail with comDE, could encode the missing comF activity needed for the final step in CoM synthesis despite independent transcription from MA3298 (comDE). The predicted protein structure model of MA3299, generated using Alphafold2, provided additional assurance when SAA was successfully docked into the proposed active site using Autodock4 (FIG. 21).(47, 48) Consistent with the hypothesis that SAA would act as the substrate for the putative ComF (MA3299), we examined the various enzymes that would produce SAA using KEGG and BRENDA databases.(49, 50) Enzymatic production of SAA would be valuable due to the absence of commercially available SAA and short-lived nature of reactive aldehydes.(51) Taurine-pyruvate aminotransferase (Tpa) was chosen to produce SAA from pyruvate and taurine (FIG. 18). The tpa gene from B. wadsworthia (Q9APM5) was selected because the enzyme has been biochemically characterized (FIG. 20b).(52, 53)

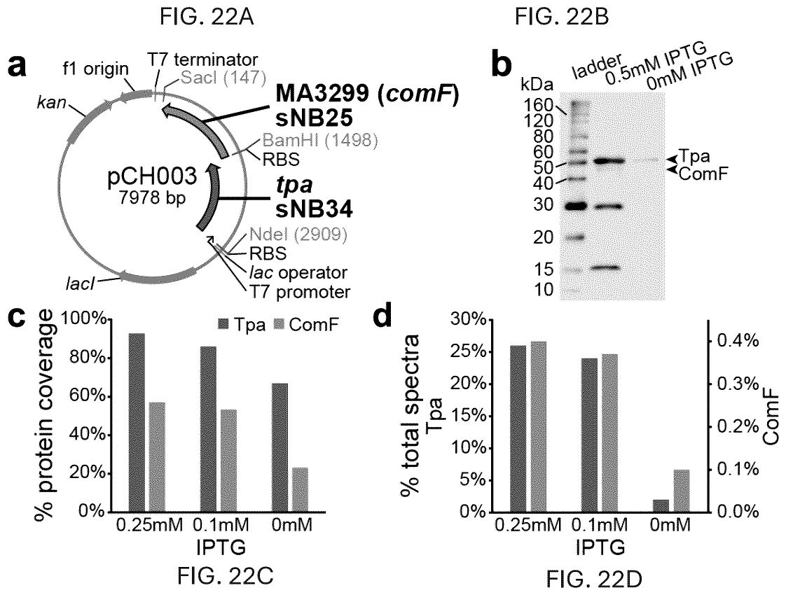

Tpa was produced recombinantly with relative ease, in stark contrast to attempts to express ComF, which failed, despite repeated efforts using a variety of IPTG concentrations, incubation conditions, E. coli strains, coexpression vectors, and media types (data not shown). Detectable production of ComF was only achieved through placing MA3299 (sNB25) directly after Q9APM5 (sNB34) in a synthetic com^{syn} operon on plasmid pCH003 (FIG. 22a).

### In Vivo Com^{syn} Expression and In Vitro CoM Production

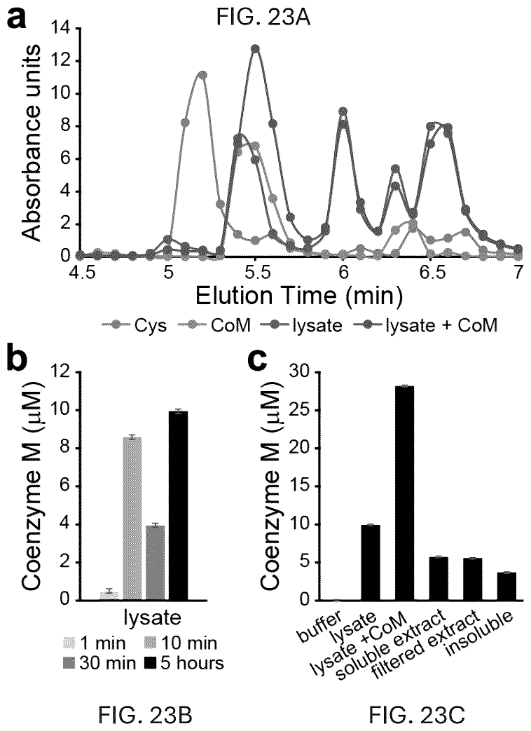

E. coli was cotransformed with pCH003 and pDB1282 to promote FeS cluster assembly in ComF and coexpression of ComF with Tpa was confirmed by Western blot (FIG. 22b) and LC-MS/MS (FIG. 22c-d). An in vitro assay was developed to measure CoM synthesis in E. coli lysates. Cells harboring pCH003 and pDB1282 plasmids were grown in defined medium, harvested by centrifugation and lysed by sonication anaerobically to preserve catalytic activity. Anaerobicity was predicted to be important due to the presence of two predicted FeS clusters in ComF. The enzymatic assay was carried out in an anaerobic chamber at 37° C. as described in the Methods. After derivatization with mBBr, products were analyzed using RP-HPLC. CoM was detected in cell lysates (FIG. 23a) confirming that ComF catalyzes SAA to CoM and that Tpa can be used in conjunction with ComF to establish an inducible metabolic pathway from pyruvate and taurine to CoM. CoM levels increased with time when lysates were supplied, but only a small amount of CoM could be detected when protein was omitted, suggesting that under the assay conditions abiotic CoM synthesis was complete within a minute, while Tpa and ComF were able to catalyze product formation over several hours (FIG. 23b). Enzyme activity was significantly decreased when lysates were removed from the anaerobic chamber to separate soluble and insoluble fractions, suggesting that our Oakridge bottle was insufficient in preventing oxygen exposure and that this exposure was detrimental to either Tpa, ComF, or both (FIG. 23c).

In Vivo Production of CoM Protects E. coli from ROS

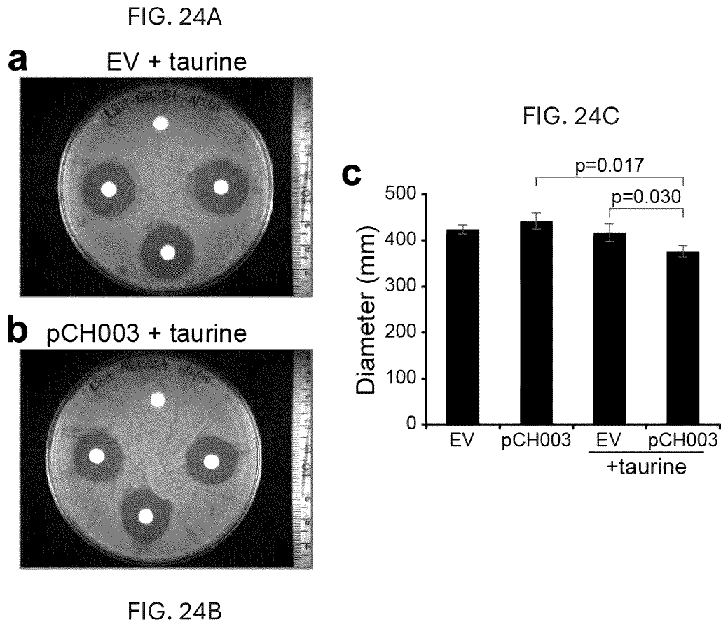

To test our hypothesis that CoM produced in vivo from the com^{syn} operon could protect cells from ROS, we designed acute and chronic oxidative stress assays. A ΔtauD λDE3 mutant strain (NB510) was created for these assays to ensure the taurine supplemented into the culture medium would not be rapidly degraded. Cells transformed with either empty vector (vector only control, VOC, NB515) or plasmid expressing the com^{syn} operon (pCH003, NB525) were grown in defined medium supplemented with 0.1 mM taurine, and com^{syn} expression was induced with 0.1 mM IPTG. The acute stress test was modelled after the Kirby Bauer Test in which H₂O₂ was applied to sterile filter disks in a lawn of cells. The zones of growth inhibition around the filter disks were decreased in ΔtauD com^{syn} plates with taurine supplementation, as compared to the VOC strain regardless of taurine supplementation indicating that E. coli expressing com^{syn} was more resistant to ROS in a taurine-dependent manner (FIG. 24).

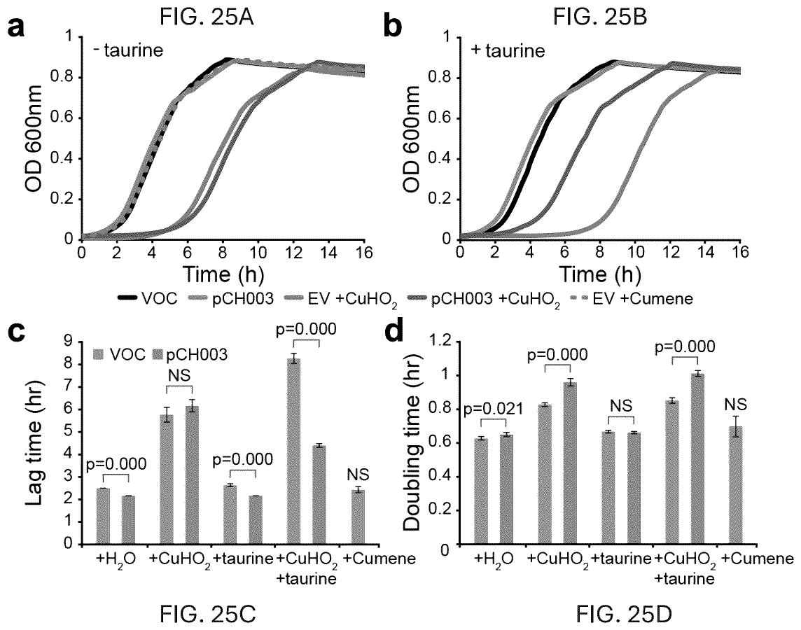

To assess resistance to ROS stress in liquid medium, cells were grown in a 96-well plate format with or without CuHO₂. Similar lag times were observed for the vector-only control strain as compared to com^{syn} expressing cells in the absence of CuHO₂ (FIG. 25a-c). Interestingly, the lag time increased for the vector-only control strain when taurine was supplied. However, the lag time decreased with taurine supplementation when cells expressed our com^{syn} operon. Observed increases in the doubling times of com^{syn} expressing cells are expected as these cultures are being tasked with recombinantly producing enzymes which the vector-only control carrying cells are not (FIG. 8d). The results from both the acute stress assay on agar plates and the chronic stress assay in liquid culture show that the CoM produced via the com^{syn} operon carried on pCH003 has an antioxidant effect and improves the ability of E. coli cells to survive peroxide stress.

### Discussion

Determination of comF in M. acetivorans

Previous investigations did not convincingly implicate MA3299 as comF in M. acetivorans.(54) The candidate comF in Methanocaldococcus jannaschii, MJ1681, shares a low 41% identity and 57% similarity with MA3299 and this level of conservation is not sufficient in and of itself to ascribe homologous function.(55) MJ1681 is missing a cysteine residue we believe to be catalytically relevant for the synthesis of CoM implying a difference in activity (Supplementary Information). Additionally, it has been proposed that the final step in CoM synthesis in methanogens is chemically catalyzed, because a knock-out of the Methanococcus maripaludis homolog of MJ1681, MMP1603, did not need nor respond to CoM in the culture medium.(54) It had also been hypothesized that there could be additional unknown intermediate products generated in the pathway between SAA and CoM. However, our docking simulations strongly suggest that MA3299 could accept SAA as a substrate.(56) The structural model of MA3299 generated using Alphafold2 and Autodock4 revealed the potential for two ferredoxin 4Fe4S cluster domains involving eight cysteine residues and an electropositive pocket that can computationally dock SAA (FIG. 21).(47, 48) This structural reasoning together with the genomic proximity to comDE were two indications that MA3299 encoded comF. Further discussion of the Alphafold model, conserved residues, features of the protein, and phylogeny can be found in the Supplementary Information.

### Features of the Com^{syn} Operon

To create the com^{syn} operon we paired two enzymes from different anaerobic organisms that inhabit drastically different environments to create a novel metabolic pathway for CoM biosynthesis. A key feature of the com^{syn} CoM biosynthesis pathway is that it begins with the universal high-flux metabolite, pyruvate, and the inexpensive non-proteinogenic amino acid taurine. While pyruvate is of high abundance in cells, taurine metabolism is not as widespread, and in many organisms CoM synthesis would depend on supplementation of taurine into the medium at relatively low cost.

The first enzyme in the com^{syn} pathway, Tpa, was obtained from Bilophila wadsworthia, a strictly anaerobic gram-negative bacterium found in the lower GI tract of humans. This bacterium is notable for its ability to utilize taurine among other sulfonates as electron acceptors for anaerobic respiration.(52) Tpa makes for an attractive first step in our pathway for several reasons; taurine is of high abundance in bile and can be found in the GI tracts of animals and, due to this, taurine import mechanisms exist in E. coli.(57, 58) Taurine is also a carbon and energy source for prokaryotic organisms in ocean sediments, where M. acetivorans was first isolated.(59) Taurine is not required by M. acetivorans, and whether the organism can catabolize taurine is unknown. The second and last step of the com^{syn} pathway is performed by ComF, an enigmatic ferredoxin-like protein obtained from the archaeal methanogen, M. acetivorans. Despite the fact that both Tpa and ComF originated in strictly anaerobic organisms, we observed that com^{syn} expression protected E. coli from ROS even when growing aerobically, suggesting the intracellular environment of aerobic organisms can maintain the stability and activity of ComF, assemble the FeS clusters in ComF, and establishes that ComF is able to accept substrate and reducing equivalents from the E. coli host via unknown endogenous S and electron donor(s).

### Taurine and CoM Import

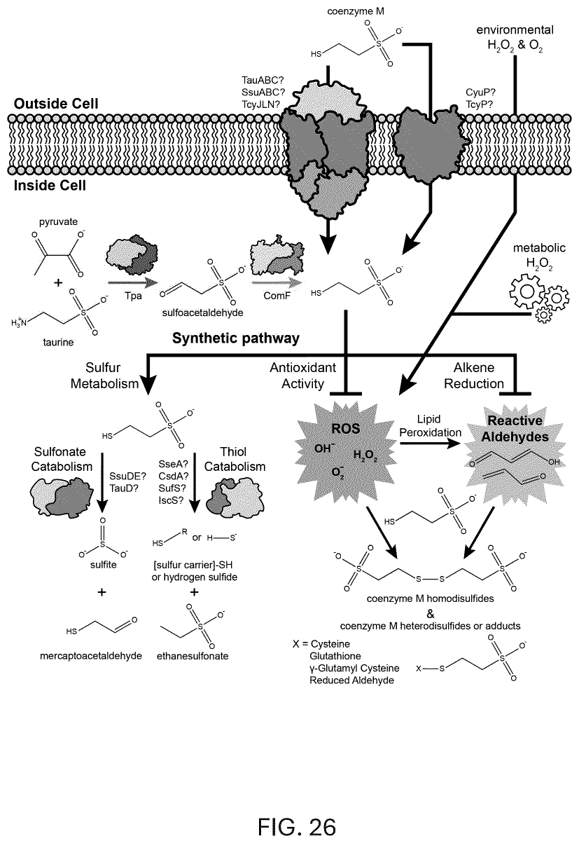

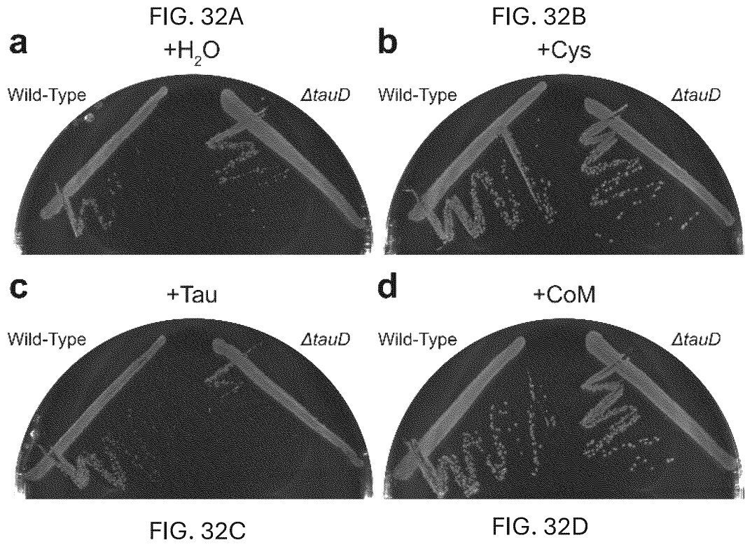

CoM is one of the smallest LMW thiols, only slightly heavier than cysteine, and can serve as a nutritional source of sulfur. We were unable to find examples in the literature for CoM transport by bacteria but our results demonstrate that CoM is imported and used as a source of sulfur by E. coli (FIG. 26, FIG. 32). In E. coli, there are two operons encoding organic sulfonate sulfur assimilation systems, ssuEADCB and tauABCD. It is possible that CoM is imported through the ATP-binding cassette (ABC) transporters SsuABC or TauABC, two systems that function to bind and bring alkanesulfonates into the cell. The binding affinities of TauABC and SsuABC toward CoM have not been investigated. However, the binding site of TauA has been described as lined with charged amino acids, while the binding site of SsuA has been described as both voluminous and hydrophobic (60). While these observations have a structural basis, they corroborate a physiological study, which observed a broad overlap in the selectivity of SsuABC and TauABC for a variety of sulfonates.(60, 61) The key difference between the two importers is that TauA lacks the ability to import hydrophobic sulfonates while SsuABC lacks the ability to import taurine.(62) It seems reasonable that either or both SsuABC and TauABC may be capable of importing CoM, although this hypothesis remains to be tested. The tauD gene encodes an α-ketoglutarate dependent taurine dioxygenase to remove sulfite from taurine using molecular oxygen and a Fe²⁺ cofactor.(63) However, there have been conflicting reports on whether E. coli ΔtauD is still able to catabolize taurine via the SsuD enzyme.(39, 43) Because we are using E. coli K-12 (JW0360) we assumed that taurine would still be digested by ΔtauD cells, however it would still be beneficial to remove one of the enzymes responsible for taurine digestion. For highest production of CoM and protection from ROS, we predict cells would need to express a sulfonate transporter and may require a mutation, such as ΔtauD, ΔssuD, or both, that would hinder the organism's ability to degrade both the substrate, taurine, and the product, CoM.

### Potential Uses of the Com^{syn} Operon

The formation of ROS can be viewed as a natural consequence of oxidative metabolism, an inevitability for which organisms have developed a multitude of proteins and antioxidant molecules to mitigate the damage caused by these unstable molecules.(64) ROS can hinder growth and replication, thus the introduction of a biosynthetic pathway for the production of CoM represents a novel mechanism to alleviate ROS stress by complementing the endogenous antioxidant systems. CoM possesses a lower redox potential than cystine (−271 mV compared to −245 mV, respectively), making it comparable to glutathione in reducing potential (−262 mV).(65, 66) The com^{syn} pathway has the potential to buffer the redox state of a wide diversity of organisms and thus may be useful for a variety of practical applications.

CoM has been used as a chemotherapy and surgery adjuvant under the pharmaceutical name Mesna for decades because of its antioxidative effects and role as a Michael donor in the detoxification of acrolein and, possibly, other alkene-containing reactive aldehydes generated through lipid peroxidation such as malondialdehyde and trans-4-hydroxy-2-nonenal.(67-72) Endogenous production of CoM by engineered microbiome bacteria has potential to detoxify pathogenesis-related factors and xenobiotics in the gastrointestinal tract or have increased resistance to the ROS based defense mechanisms of neutrophils allowing pathogenic species to be selectively eliminated.(73) CoM production by E. coli or other bacteria creates exciting opportunities to bolster agriculture. The com^{syn} substrate taurine is nontoxic to humans, can act as an osmoprotectant for microbes, and can be metabolized by microbes, plants, and humans.(74, 75) Thus, aquaculture systems which routinely supply taurine to fish in their feed could be converted to CoM by freshwater microbes to improve animal health. Enhanced CoM production in Streptomyces spp. has potential for improving synthesis of polyunsaturated fats or allow bacteria to efficiently detoxify heavy metals.(76-79) Because CoM naturally serves as a methyl carrier in methanogenic archaea, CoM could feasibly be used for methylation and methyl transfer reactions in conjunction with (synthetic) corrinoid proteins or other tetrapyrrole catalysts, for example to methylate phenols.(80) Finally, complementing the endogenous antioxidant systems by buffering redox metabolism in any cell type, whether archaeal, bacterial, animal, or plant, has broad potential to promote growth rates and biosynthetic productivity of any organism used in agriculture, health, and biomanufacturing.

### Supplemental Information

MA3299 (ComF) is currently annotated as a methanogenesis marker protein with ferredoxin binding domains. Rauch et al. identified a subsection of the protein family COG1900, COG1900d, found almost exclusively in methanogens and characterized by a C-terminal domain containing two 4Fe-4S clusters.(1) Perona et al. identified the that MA3299 was a member of this protein family and proposed that its function may be related to CoM and CoB biosynthesis, although the rationale was not described.(2) However, prior studies have indicated that an MA3299 homolog in Methanococcus maripaludis, Mmp1603, is a nonessential gene.(3) Therefore in vivo physiology studies cast doubt on the idea that MA3299 could be involved in CoM biosynthesis. It remains possible that a ComF enzyme catalyzes the final step in CoM biosynthesis, but it has been suggested that the reaction itself could occur at a high enough rate autocatalytically to provide methanogens with enough CoM to facilitate methanogenesis in the absence of a ComF enzyme.(4) To assess whether MA3299 was capable of catalyzing a ComF reaction we analyzed protein structures generated in Pymol.(5)

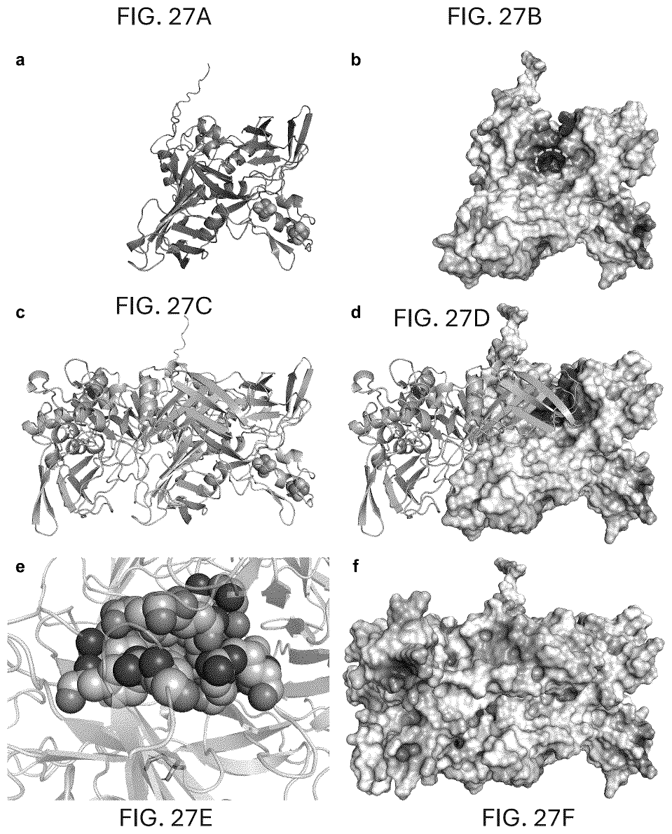

Using AlphaFold2, the MA3299 protein structure was simulated using the University of Nebraska Holland Computing Center.(6) This simulated structure is also publicly available in the AlphaFold Protein Structure Database, with the majority of the protein marked as being modelled with a “very high” confidence (Accession: Q8TKU5).(7, 8) Autodock4 was used to simulate interactions between sulfoacetaldehyde (SAA) or a Fe—S cluster and MA3299.(9) We were able to dock two Fe—S clusters within an arm of MA3299, while SAA was docked into the center of the protein (FIG. 27a). The Pymol APBS Electrostatics plugin revealed that SAA is nestled into the deepest portion of an electropositive pocket (FIG. 27b). ColabFold v1.5.5 was used to generate a dimerized version of MA3299.(10) When simulated as a dimer, the large opening to the electropositive pocket in which SAA rests is obscured by the dimerization arm of MA3299 (FIG. 27c,d,f).

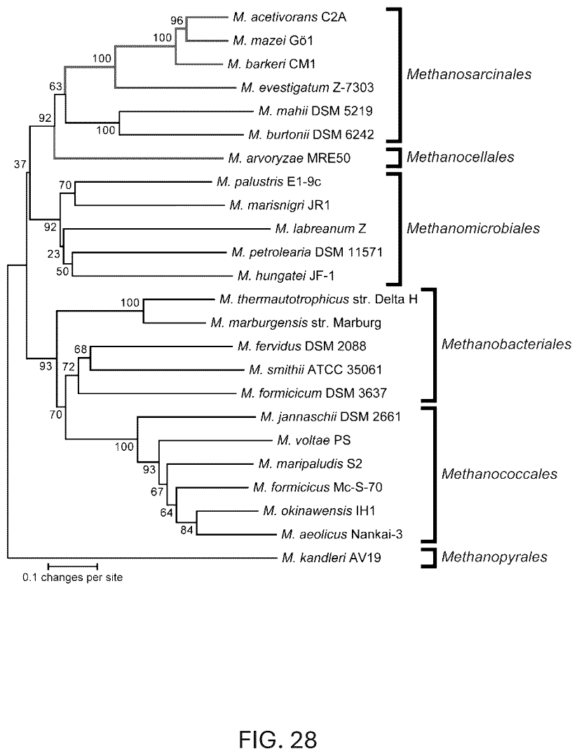

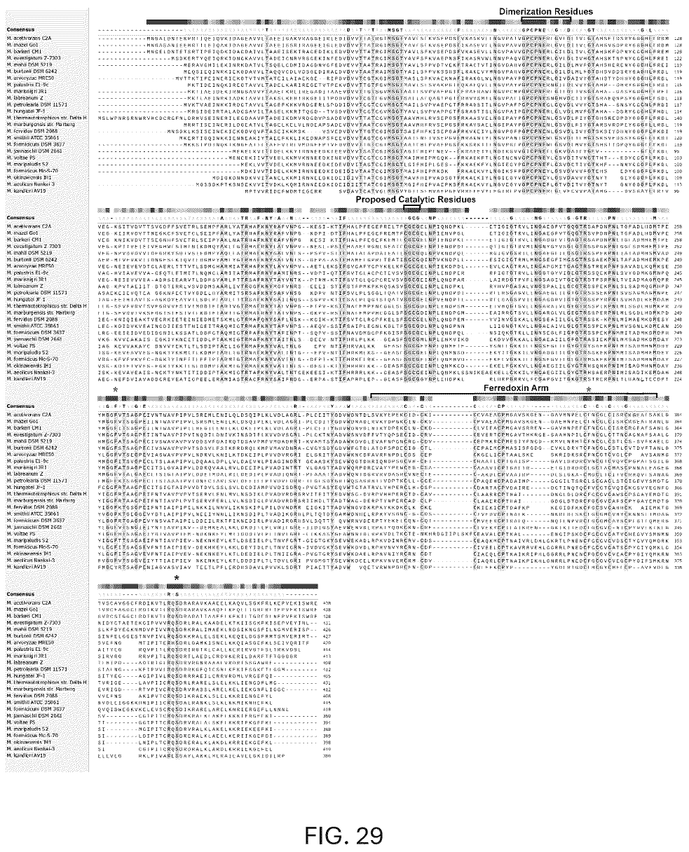

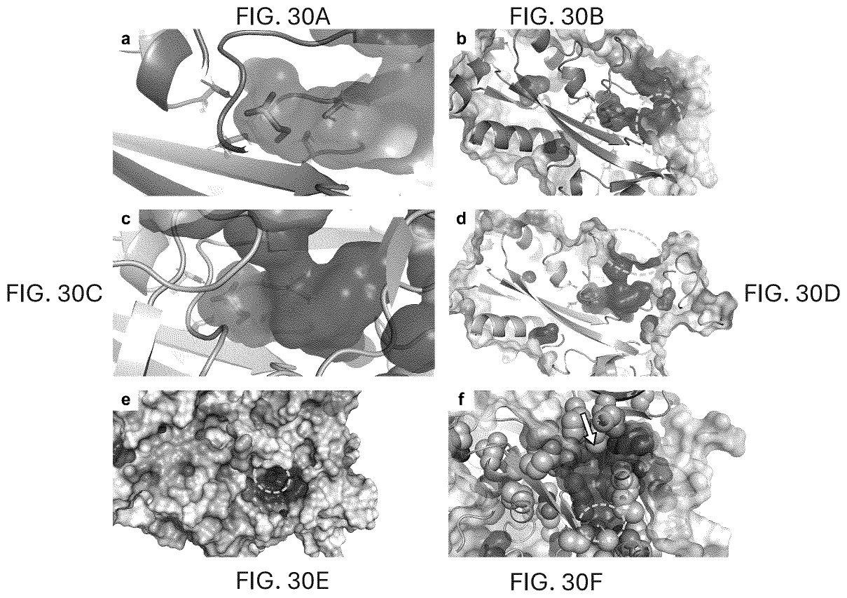

To assess the potential functions of conserved residues of MA3299, we identified homologous proteins using the Basic Local Alignment Search Tool (BLAST).(11) Twenty-two amino acid sequences were chosen as a representative sample of the homologs available from eleven Class I and Class II methanogens. Sequences were aligned using the MUSCLE algorithm and a Neighbor-Joining phylogenetic tree was assembled with MEGA11: Molecular Evolutionary Genetics Analysis version 11 (FIG. 28).(12, 13) When assembled into a phylogenetic tree, a clear delineation between Class I (orders of Methanobacteriales, Methanococcales, and Methanopyrales) and Class II (orders of Methanosarcinales, Methanocellales, and Methanomicrobiales) methanogens were observed. SnapGene software (www.snapgene.com) was used to process the same sequence alignment and highlight the residues which were conserved at a rate of over 95% (FIG. 29).(14) We observed that 68 out of 438 amino acid residues are conserved at this rate, including residues we consider integral to the dimerization arm, catalysis, and the cysteine residues within the ferredoxin arm. Examination of the dimer model with conserved residues exposed, revealed a stretch of highly conserved residues on the portion of the dimerization arm that obscures the active site (GPCPNEXXGXXD (SEQ ID NO: 64), along with a variety of conserved residues scattered throughout the sequence that form the entrance to the active site (FIG. 27e). The APBS Electrostatic simulations of the monomer and dimer allowed us to examine the shape and structure of the electropositive pocket in which SAA binds (FIG. 30).(15) In the monomeric version of ComF there is a large, open area available for interaction (FIG. 30a,b), however the dimeric version obscures this opening allowing a much smaller opening to appear in a different location (FIG. 30c,d,e). Interestingly, one of the residues blocking the large opening to the active site in the dimeric version of the protein is Cys95, a highly conserved residue conserved in the COG1900a family of proteins. This interference with the entrance is more easily observed when the electrostatic surface is displayed for only one of the two side chains in this simulation (FIG. 30f).

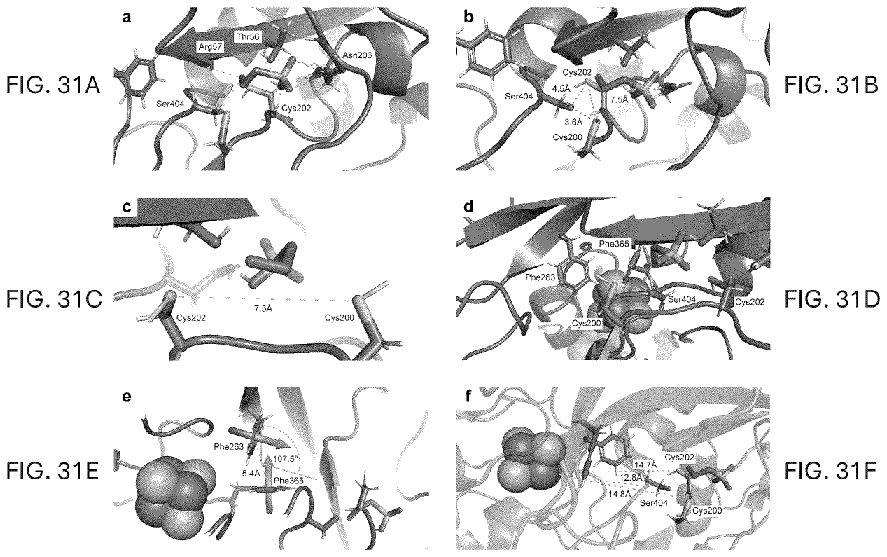

Examination of the active site indicates that a number of polar contacts form between ComF and the docked SAA (FIG. 31a). SAA possesses a sulfonate group, which forms polar contacts with the amino group of Cys202 and the side chains of Thr56 and Asn206. The aldehyde group of SAA forms polar contacts with carboxyl group of Arg57 and the side chains of Thr56 and Ser404; two cysteine residues flank either side of the aldehyde group of SAA, Cys200 and Cys202. The arrangement of the three residues around the aldehyde functional group is notable as Ser404 is positioned between the two cysteine residues with a distance of 3.6 Å and 4.5 Å between the oxygen of the Ser404 side chain and the sulfurs of Cys200 and Cys202 respectively, while the distance between the sulfurs is 7.5 Å (FIG. 31b-c). All residues labeled on FIG. 31 are highly conserved with the exception of one, Cys200, which among all Class I

methanogens examined, is a highly conserved serine, while Class II methanogens have a mixture of serine and cysteine residues at this position (sequences with cysteine marked with a red branch in FIG. 28). In our model, when Cys200 and the closest Fe—S cluster are placed in line with one another, two phenylalanine residues, Phe263 and Phe365, can be observed between them (FIG. 31d). The arrangement of Phe263 and Phe365, conserved in 97% and 81% of sequences respectively, is indicative of an edge-to-face T-shaped π-π interaction. The centroid distance between these phenylalanine residues is 5.4 Å, and the angle between the two residues is 72.5° (180°-107.5°), both of which fall within the reported measurements of π-π interactions between aromatic residues (FIG. 31e).(16) This type of interaction is not uncommon in proteins and can serve a structural purpose, such as keeping the ferredoxin arm close to the body of the protein.(17)

Robert White attributed the function of ComF to MJ1681 in Methanocaldococcus jannaschii, which shares 41.34% identity with MA3299.(4) In this paper, it is hypothesized that the reaction mechanism by which a sulfur is added to SAA involves two cysteine residues that form a disulfide bond. Examination of the active sites of the Alphafold constructs of both MA3299 and MJ1681 raise questions about this mechanism of catalysis. There are two cysteine residues nearby one another within the active site pocket of MJ1681, Cys36 (Arg57 in MA3299) and Cys166 (Cys202 in MA3299), however Cys36 is not well conserved across different species of methanogens and the side chain faces the opposite direction of Cys166. The two cognizant conserved cysteines of MA3299 are located near one another and separated by a single glycine residue, which allows for a higher degree of backbone flexibility. However, the distance between their sulfur atoms, 7.5 Å, indicates that a bond between the two would be unlikely. The high conservation of Cys202 and proximity to the aldehyde group of SAA in the active site may indicate a catalytic purpose. The same could be stated for Cys/Ser200 which share the same general size but differ in their functional group; sulfur and oxygen are both chalcogens meaning that they have similar electron configurations, but sulfur is less electronegative than oxygen. This difference could impact the catalytic efficacy of CoM production through electron affinities, atomic diameter, and bond lengths.

Considering that our MA3299 protein structure was artificially constructed and that there is a degree of flexibility in proteins, the predicted distance between Cys200 and Cys202 (7.5 Å) can help inform a potential reaction mechanism involving the formation of a trisulfide. Sulfur is required for the synthesis of CoM from SAA and there are several potential sources of a sulfur donation: MA1715 is a sulfur donor for MA1821, MA3300 is a protein with homology to MoaD/ThiS proteins with high proximity to MA3299 (FIG. 21b), or the source could be from hydrogen sulfide itself.(18-20) The atomic radius of a sulfur atom is 1A and a sulfur-sulfur (S—S) bond is considered to be flexible with bond lengths ranging from 1.8-3 Å.(21, 22) If the distance between Cys200 and Cys202 is 7.5 Å then between a 1 Å radius from each cysteine sulfur in addition to a 2 Å diameter of a sulfur atom situated between them leaves 4.5 Å for bonds. If there are two S—S bonds then the space remaining leaves 2.25 Å for each bond, consistent with the observed S—S bond length ranges reported depending on the angle of the bonds and flexibility of the peptide backbone. Ser404 is an additional interesting feature which could either coordinate the aldehyde group of SAA or act as an additional point of contact for the sulfur reagent. Regardless of the exact mechanism, we are confident that catalysis would require electron donation from the Fe—S clusters. The residues we hypothesize establish the catalytic site rest between 13-15 Å from the nearest Fe—S cluster placing it near the limit of effective electron tunnelling (FIG. 31f).(23) The nearby t-stacked phenylalanine residues suggest a mechanism which could assist this electron tunnelling.

TABLE 6
Strains, primers, and genetic parts used in this study.
Primers

| oNB# | 5′→3′ | Purpose | Source |
|---|---|---|---|
| 924 | Tcatatgcacctccttcttaaagttaaacaaaattatttc | Amplification of | This study |
| | (SEQ ID NO: 44) | pET24a for pCH003 | |
| 925 | Tgatgagctcactgagatccggctgctaac (SEQ ID | Amplification of | This study |
| | NO: 45) | pET24a for pCH003 | |
| 926 | Catcggatcctcctcatcaatgatgatgg (SEQ ID | Amplification of | This study |
| | NO: 46) | sNB34 for pCH003 | |
| 927 | Taagaaggaggtgcatatgacctatgataaag (SEQ | Amplification of | This study |
| | ID NO: 47) | sNB34 for pCH003 | |
| 928 | Ggatctcagtgagctcatcagtgatgatgatg (SEQ | Amplification of | This study |
| | ID NO: 48) | sNB25 for pCH003 | |
| 929 | Ttgatgaggaggatccgatgaatggtgc (SEQ ID | Amplification of | This study |
| | NO: 49) | sNB25 for pCH003 | |

Plasmids

| Name | Feature and Use | Source |
|---|---|---|
| pET24a | Vector backbone for moderate copy number and kanamycin | Novagen |
| | resistance cassette | |
| pCH003 | Expression vector for enzymes Tpa (sNB34) and ComF (sNB25) | This study |
| | derived from pET24a | |
| pCH019 | Expression vector for enzyme ComF (sNB25) derived from pET24a | This study |
| pCH020 | Expression vector for enzyme Taurine-pyruvate aminotransferase | This study |
| | (sNB34) derived from pET24a | |
| pCP20 | Expression vector for FLP recombinase to remove kanamycin | (28) |
| | resistance from Keio strains | |
| pDB1282 | Expression vector for the isc operon from Azotobacter vinelandii, | (81) |
| | to increase biosynthesis of Fe-S clusters and their incorporation into | |
| | proteins | |

Strains

| NB# | Genotype | Characteristics | Source |
|---|---|---|---|
| NB003 | F′ proA+B+ lacIq Δ(lacZ) | DH5α. E. coli for | New |
| | M15 zzf:: Tn10 (TetR)/ | plasmid cloning and | England |
| | fhuA2Δ(argF-lacZ)U169 phoA glnV44 | propagation | Biolabs |
| | Φ80Δ(lacZ)M15 gyrA96 recA1 endA1 | | |
| | thi-1 hsdR17 | | |
| NB008 | MiniF lysY lacIq(CamR)/fhuA2 | BL21 E. coli for | New |
| | lacZ::T7 gene1 [[Ion]] ompT gal sulA11 | protein | England |
| | R(mcr-73::miniTn10-TetS)2 [[dcm]] | overexpression | Biolabs |
| | R(zgb-210::Tn10-TetS) endA1 Δ(mcrC- | | |
| | mrr)114::IS10 | | |
| NB398 | F−, Δ(araD-araB)567, ΔlacZ4787(::rrnB- | E. coli K-12 | Coli Genetic |
| | 3), λ−, rph-1, Δ(rhaD-rhaB)568, hsdR514 | wild-type | Stock Center |
| | | | (CGSC) |
| | | | BW25113 |
| NB494 | F−, Δ(araD-araB)567, ΔlacZ4787(::rrnB- | Kanamycin resistant | Coli Genetic |
| | 3), λ−, rph-1, Δ(rhaD-rhaB)568, | E. coli K-12 with | Stock Center |
| | hsdR514, ΔtauD736::kan | taurine dioxygenase | (CGSC) |
| | | deletion (ΔtauD) | JW0360-3 |
| NB499 | F−, Δ(araD-araB)567, ΔlacZ4787(::rrnB- | Derivative of NB494 | This study |
| | 3), λ−, rph-1, Δ(rhaD-rhaB)568, | via FLP recombinase | |
| | hsdR514, ΔtauD736 | to remove the | |
| | | kanamycin resistance | |
| | | cassette | |
| NB506 | F−, Δ(araD-araB)567, ΔlacZ4787(::rrnB- | Lysogenized DE3 λ | This study |
| | 3), λ−, rph-1, Δ(rhaD-rhaB)568, | phage derivative of | |
| | hsdR514, λ(DE3) | NB398 to express T7 | |
| | | polymerase | |
| NB510 | F−, Δ(araD-araB)567, ΔlacZ4787(::rrnB- | Lysogenized DE3 λ | This study |
| | 3), λ−, rph-1, Δ(rhaD-rhaB)568, | phage derivative of | |
| | hsdR514, ΔtauD736, λ(DE3) | NB499 to express T7 | |
| | | polymerase | |
| 515 | F−, Δ(araD-araB)567, ΔlacZ4787(::rrnB- | ΔtauD vector-only | This study |
| | 3), λ−, rph-1, Δ(rhaD-rhaB)568, | control | |
| | hsdR514, ΔtauD736, λ(DE3)/pET24a | | |
| 521 | F−, Δ(araD-araB)567, ΔlacZ4787(::rrnB- | com^{syn} | This study |
| | 3), λ−, rph-1, Δ(rhaD-rhaB)568, | | |
| | hsdR514, λ(DE3)/pCH003 | | |
| 525 | F−, Δ(araD-araB)567, ΔlacZ4787(::rrnB- | ΔtauD com^{syn} | This study |
| | 3), λ−, rph-1, Δ(rhaD-rhaB)568, | | |
| | hsdR514, ΔtauD736, λ(DE3)/pCH003 | | |

TABLE 7
Defined Media Components.

| | | Concentration | |
|---|---|---|---|
| Chemical | Formula | (mM) | Reference |
| Potassium Phosphate | K₂HPO₄ | 45.9 | (24) |
| (dibasic) | | | |
| Potassium Phosphate | KH₂PO₄ | 33.1 | (24) |
| (monobasic) | | | |
| Sodium Citrate | Na₃C₆H₅O₇ | 1.7 | (24) |
| Casamino Acids | | 0.2% | (25) |
| L-Tryptophan | C₁₁H₁₂N₂O₂ | 0.5 | (25) |
| Ammonium Sulfate | NH₄SO₄ | 7.5711 | (24) |
| Magnesium Sulfate | MgSO₄ | 1 | (26) |
| Calcium Chloride | CaCl₂ | 0.1 | (26) |
| Glucose | C₆H₁₂O₆ | 0.2% | (25, 26) |
| Thiamine | | 0.003 | (26, 27) |
| Biotin | | 0.0041 | (26) |
| Ethylenediaminetetraacetic | C₁₀H₁₆N₂O₈ | 0.134 | (28) |
| acid (EDTA) | | | |
| Iron(II) sulfate | FeSO₄ | 0.031 | (28) |
| Zinc chloride | ZnCl₂ | 0.0062 | (28) |
| Copper(II) chloride | CuCl₂ | 0.00076 | (28) |
| Cobalt(II) chloride | CoCl₂ | 0.00042 | (28) |
| Boric acid | H₃BO₃ | 0.00162 | (28) |
| Manganese(II) chloride | MnCl₂ | 0.000081 | (28) |

TABLE 8
Accession numbers (NCBI) for proteins of the COG1900d family.

| Host Genome | Accession Number |
|---|---|
| Methanosarcina acetivorans C2A | AAM06669.1 |
| Methanosarcina mazei Go1 | AAM29831.1 |
| Methanosarcina barkeri CM1 | AKJ37213.1 |
| Methanohalobium evestigatum Z-7303 | ADI73780.1 |
| Methanohalophilus mahii DSM 5219 | ADE37251.1 |
| Methanococcoides burtonii DSM 6242 | ABE52367.1 |
| Methanocella arvoryzae MRE50 | CAJ37933.1 |
| Methanosphaerula palustris E1-9c | ACL17998.1 |
| Methanoculleus marisnigri JR1 | ABN56045.1 |
| Methanocorpusculum labreanum Z | ABN06676.1 |
| Methanolacinia petrolearia DSM 11571 | ADN35058.1 |
| Methanospirillum hungatei JF-1 | ABD42665.1 |
| Methanothermobacter thermautotrophicus str. ΔH | AAB86156.1 |
| Methanothermobacter marburgensis str. Marburg | ADL57871.1 |
| Methanothermus fervidus DSM 2088 | ADP76951.1 |
| Methanobrevibacter smithii ATCC 35061 | ABQ86290.1 |
| Methanobacterium formicicum DSM 3637 | EKF85189.1 |
| Methanocaldococcus jannaschii DSM 2661 | AAB99702.1 |
| Methanococcus voltae PS | MCS3921535.1 |
| Methanococcus maripaludis S2 | CAF31159.1 |
| Methanotorris formicicus Mc-S-70 | EHP87423.1 |
| Methanothermococcus okinawensis IH1 | AEH06645.1 |
| Methanococcus aeolicus Nankai-3 | ABR56944.1 |
| Methanopyrus kandleri AV19 | AAM01573.1 |

### REFERENCES FOR EXAMPLE 2

1. Sies H, Berndt C, & Jones D P (2017) Oxidative stress. Annual review of biochemistry 86(1):715-748.
2. Lu Z & Imlay J A (2021) When anaerobes encounter oxygen: mechanisms of oxygen toxicity, tolerance and defence. Nature reviews microbiology 19(12):774-785.
3. Graupner M, Xu H, & White R H (2000) Identification of the gene encoding sulfopyruvate decarboxylase, an enzyme involved in biosynthesis of coenzyme M. Journal of Bacteriology 182(17):4862-4867.
4. Lobo V, Patil A, Phatak A, & Chandra N (2010) Free radicals, antioxidants and functional foods: Impact on human health. Pharmacognosy reviews 4(8):118.
5. Wehrs M, et al. (2019) Engineering robust production microbes for large-scale cultivation. Trends in microbiology 27(6):524-537.
6. Chevallier V, Andersen M R, & Malphettes L (2020) Oxidative stress-alleviating strategies to improve recombinant protein production in CHO cells. Biotechnology and bioengineering 117(4):1172-1186.
7. Harada M, et al. (2021) Evolution of superoxide dismutases and catalases in cyanobacteria: Occurrence of the antioxidant enzyme genes before the rise of atmospheric oxygen. Journal of Molecular Evolution 89:527-543.
8. He H, et al. (2021) An abiotic source of Archean hydrogen peroxide and oxygen that pre-dates oxygenic photosynthesis. Nature Communications 12(1):6611.
9. Haqq-Misra J, Kasting J F, & Lee S (2011) Availability of 02 and H₂O₂ on pre-photosynthetic Earth. Astrobiology 11(4):293-302.
10. Flint D H, Tuminello J, & Emptage M (1993) The inactivation of Fe—S cluster containing hydro-lyases by superoxide. Journal of Biological Chemistry 268(30):22369-22376.
11. Fahey R C (2013) Glutathione analogs in prokaryotes. Biochimica et Biophysica Acta (BBA)-General Subjects 1830(5):3182-3198.
12. Lu S C (2009) Regulation of glutathione synthesis. Molecular aspects of medicine 30(1-2):42-59.
13. Helbig K, Bleuel C, Krauss G J, & Nies D H (2008) Glutathione and transition-metal homeostasis in Escherichia coli. Journal of bacteriology 190(15):5431-5438.
14. Janssen D B, Dinkla I J, Poelarends G J, & Terpstra P (2005) Bacterial degradation of xenobiotic compounds: evolution and distribution of novel enzyme activities. Environmental microbiology 7(12):1868-1882.
15. Lee C & Park C (2017) Bacterial responses to glyoxal and methylglyoxal: reactive electrophilic species. International journal of molecular sciences 18(1):169.
16. Newton G L, et al. (1996) Distribution of thiols in microorganisms: mycothiol is a major thiol in most actinomycetes. Journal of bacteriology 178(7):1990-1995.
17. Pivato M, Fabrega-Prats M, & Masi A (2014) Low-molecular-weight thiols in plants: Functional and analytical implications. Archives of biochemistry and biophysics 560:83-99.
18. Gressier B, et al. (1995) Scavenging of reactive oxygen species by letosteine, a molecule with two blocked-SH groups: Comparison with free-SH drugs. Pharmacy World and Science 17:76-80.
19. Mashiach E, et al. (2001) Mesna: a novel renoprotective antioxidant in ischaemic acute renal failure. Nephrology Dialysis Transplantation 16(3):542-551.
20. Buan N R & Metcalf W W (2010) Methanogenesis by Methanosarcina acetivorans involves two structurally and functionally distinct classes of heterodisulfide reductase. Mol Microbiol 75(4):843-853.
21. Salvi A M, Hines C J, & Buan N R (Unpublished.
22. Jasso-Chavez R, et al. (2015) Air-adapted Methanosarcina acetivorans shows high methane production and develops resistance against oxygen stress. PLoS One 10(2):e0117331.
23. Graham D E, Xu H, & White R H (2002) Identification of coenzyme M biosynthetic phosphosulfolactate synthase: a new family of sulfonate-biosynthesizing enzymes. Journal of Biological Chemistry 277(16):13421-13429.
24. Graham D E, Graupner M, Xu H, & White R H (2001) Identification of coenzyme M biosynthetic 2-phosphosulfolactate phosphatase. A member of a new class of Mg2+-dependent acid phosphatases. European Journal of Biochemistry 268(19):5176-5188.
25. Graupner M, Xu H, & White R H (2000) Identification of an archaeal 2-hydroxy acid dehydrogenase catalyzing reactions involved in coenzyme biosynthesis in methanoarchaea. Journal of Bacteriology 182(13):3688-3692.
26. Helgadóttir S, Rosas-Sandoval G, Söll D, & Graham D E (2007) Biosynthesis of phosphoserine in the Methanococcales. Journal of bacteriology 189(2):575-582.
27. Graham D E, Taylor S M, Wolf R Z, & Namboori S C (2009) Convergent evolution of coenzyme M biosynthesis in the Methanosarcinales: cysteate synthase evolved from an ancestral threonine synthase. Biochemical Journal 424(3):467-478.
28. Cherepanov P P & Wackernagel W (1995) Gene disruption in Escherichia coli: TcR and KmR cassettes with the option of Flp-catalyzed excision of the antibiotic-resistance determinant. Gene 158(1):9-14.
29. Miller J (1978) H. (1972) Experiments in molecular genetics. ColdSpring Harbor Laboratory. Cold Spring Harbor, NY:328-330.
30. Anjem A, Varghese S, & Imlay J A (2009) Manganese import is a key element of the OxyR response to hydrogen peroxide in Escherichia coli. Molecularmicrobiology 72(4):844-858.
31. Monk J W, et al. (2017) Rapid and inexpensive evaluation of nonstandard amino acid incorporation in Escherichia coli. ACS synthetic biology 6(1):45-54.
32. Elbing K & Brent R (2002) Media preparation and bacteriological tools. Current Protocols in Molecular Biology 59(1):1.1. 1-1.1. 7.
33. Flamholz A I, et al. (2020) Functional reconstitution of a bacterial CO2 concentrating mechanism in Escherichia coli. Elife 9:e59882.
34. Laemmli U K (1970) Cleavage of structural proteins during the assembly of the head of bacteriophage T4. nature 227(5259):680-685.
35. Wilson C M (1983) [18] Staining of proteins on gels: Comparisons of dyes and procedures. Methods in enzymology, (Elsevier), Vol 91, pp 236-247.
36. Cicchillo R M, et al. (2004) Lipoyl synthase requires two equivalents of S-adenosyl-L-methionine to synthesize one equivalent of lipoic acid. Biochemistry 43(21):6378-6386.
37. Minocha R, Thangavel P, Dhankher O P, & Long S (2008) Separation and quantification of monothiols and phytochelatins from a wide variety of cell cultures and tissues of trees and other plants using high performance liquid chromatography. Journal of Chromatography A 1207(1-2):72-83.
38. Schneider C A, Rasband W S, & Eliceiri K W (2012) NIH Image to ImageJ: 25 years of image analysis. Nature methods 9(7):671-675.
39. Van der Ploeg J, et al. (1996) Identification of sulfate starvation-regulated genes in Escherichia coli: a gene cluster involved in the utilization of taurine as a sulfur source. Journal of bacteriology 178(18):5438-5446.
40. van der Ploeg J R, Iwanicka-Nowicka R, Bykowski T, Hryniewicz M M, & Leisinger T (1999) The Escherichia coli ssuEADCB gene cluster is required for the utilization of sulfur from aliphatic sulfonates and is regulated by the transcriptional activator Cbl. Journal of Biological Chemistry 274(41):29358-29365.
41. Eichhorn E, van der Ploeg J R, Kertesz M A, & Leisinger T (1997) Characterization of α-ketoglutarate-dependent taurine dioxygenase from Escherichia coli. Journal of Biological Chemistry 272(37):23031-23036.
42. Eichhorn E, van der Ploeg J R, & Leisinger T (1999) Characterization of a two-component alkanesulfonate monooxygenase from Escherichia coli. Journal of Biological Chemistry 274(38):26639-26646.
43. Nishikawa M, Shen L, & Ogawa Ki (2018) Taurine dioxygenase (tauD)-independent taurine assimilation in Escherichia coli. Microbiology 164(11):1446-1456.
44. Mutoh N, Kawabata M, & Kitajima S (2005) Effects of four oxidants, menadione, 1-chloro-2, 4-dinitrobenzene, hydrogen peroxide and cumene hydroperoxide, on fission yeast Schizosaccharomyces pombe. Journal of biochemistry 138(6):797-804.
45. Kurth J M, Op den Camp H J, & Welte C U (2020) Several ways one goal-methanogenesis from unconventional substrates. Applied Microbiology and Biotechnology 104(16):6839-6854.
46. EI-Mansi M (2023) Control of central metabolism's architecture in Escherichia coli: An overview. Microbiological Research 266:127224.
47. Jumper J, et al. (2021) Highly accurate protein structure prediction with AlphaFold. Nature 596(7873):583-589.
48. Morris G M, et al. (2009) AutoDock4 and AutoDockTools4: Automated docking with selective receptor flexibility. J Comput Chem 30(16):2785-2791.
49. Kanehisa M & Goto S (2000) KEGG: kyoto encyclopedia of genes and genomes. Nucleic Acids Res 28(1):27-30.
50. Schomburg I, et al. (2013) BRENDA in 2013: integrated reactions, kinetic data, enzyme function data, improved disease classification: new options and contents in BRENDA. Nucleic Acids Res 41(Database issue):D764-772.
51. Fritz K S & Petersen D R (2013) An overview of the chemistry and biology of reactive aldehydes. Free Radical Biology and Medicine 59:85-91.
52. Laue H & Cook A M (2000) Biochemical and molecular characterization of taurine: pyruvate aminotransferase from the anaerobe Bilophila wadsworthia. European Journal of Biochemistry 267(23):6841-6848.
53. Baron E J, et al. (1989) Bilophila wadsworthia, gen. nov. and sp. nov., a unique gram-negative anaerobic rod recovered from appendicitis specimens and human faeces. Microbiology 135(12):3405-3411.
54. White R H (2019) Identification of an Enzyme Catalyzing the Conversion of Sulfoacetaldehyde to 2-Mercaptoethanesulfonic Acid in Methanogens. Biochemistry 58(15):1958-1962.
55. Jeffery C J (2023) Current successes and remaining challenges in protein function prediction. Frontiers in Bioinformatics 3:1222182.
56. White R H (1986) Intermediates in the biosynthesis of coenzyme M (2-mercaptoethanesulfonic acid). Biochemistry 25(18):5304-5308.
57. van der Ploeg J R, Eichhorn E, & Leisinger T (2001) Sulfonate-sulfur metabolism and its regulation in Escherichia coli. Archives of microbiology 176(1-2):1-8.
58. Reshetnyak V I (2013) Physiological and molecular biochemical mechanisms of bile formation. World journal of gastroenterology: WJG 19(42):7341.
59. Sowers K R, Baron S F, & Ferry J G (1984) Methanosarcina acetivorans sp. nov., an acetotrophic methane-producing bacterium isolated from marine sediments. Applied and Environmental Microbiology 47(5):971-978.
60. Qu F, ElOmari K, Wagner A, De Simone A, & Beis K (2019) Desolvation of the substrate-binding protein TauA dictates ligand specificity for the alkanesulfonate ABC importer TauABC. Biochemical Journal 476(23):3649-3660.
61. Tófoli de Araujo F, et al. (2013) Structural and physiological analyses of the alkanesulphonate-binding protein (SsuA) of the citrus pathogen Xanthomonas citri. PLoS One 8(11):e80083.
62. Eichhorn E, van der Ploeg J R, & Leisinger T (2000) Deletion analysis of the Escherichia coli taurine and alkanesulfonate transport systems. Journal of Bacteriology 182(10):2687-2695.
63. Bollinger Jr J M, Price J C, Hoffart L M, Barr E W, & Krebs C (2005) Mechanism of taurine: α-ketoglutarate dioxygenase (TauD) from Escherichia coli. European journal ofinorganic chemistry 2005(21):4245-4254.
64. Imlay J A (2019) Where in the world do bacteria experience oxidative stress? Environmental microbiology 21(2):521-530.
65. Laird M G, Adlung N, Koivisto J J, & Scheller S (2023) Thiol-Disulfide Exchange Kinetics and Redox Potential of the Coenzyme M and Coenzyme B Heterodisulfide, an Electron Acceptor Coupled to Energy Conservation in Methanogenic Archaea. ChemBioChem 25(1):e202300595.
66. Millis K K, Weaver K H, & Rabenstein D L (1993) Oxidation/reduction potential of glutathione. The Journal of Organic Chemistry 58(15):4144-4146.
67. Scheulen M E, Niederle N, Bremer K, Schutte J, & Seeber S (1983) Efficacy of ifosfamide in refractory malignant diseases and uroprotection by mesna: results of a clinical phase II-study with 151 patients. Cancer Treatment Reviews 10:93-101.
68. Denaro V, et al. (2008) Effectiveness of a mucolythic agent as a local adjuvant in revision lumbar spine surgery. European Spine Journal 17:1752-1756.
69. Casale M, Di Martino A, Salvinelli F, Trombetta M, & Denaro V (2010) MESNA for chemically assisted tissue dissection. Expert Opinion on Investigational Drugs 19(6):699-707.
70. Keeney J T, et al. (2018) Doxorubicin-induced elevated oxidative stress and neurochemical alterations in brain and cognitive decline: protection by MESNA and insights into mechanisms of chemotherapy-induced cognitive impairment (“chemobrain”). Oncotarget 9(54):30324.
71. Zhu Q, Sun Z, Jiang Y, Chen F, & Wang M (2011) Acrolein scavengers: reactivity, mechanism and impact on health. Molecular nutrition & food research 55(9):1375-1390.
72. Pfohl-Leszkowicz A, et al. (2002) MESNA protects rats against nephrotoxicity but not carcinogenicity induced by ochratoxin A, implicating two separate pathways. Facta Univ. Ser. Med. Biol 9:57-63.
73. Ghaisas S, Maher J, & Kanthasamy A (2016) Gut microbiome in health and disease: Linking the microbiome-gut-brain axis and environmental factors in the pathogenesis of systemic and neurodegenerative diseases. Pharmacology & therapeutics 158:52-62.
74. Singh P, et al. (2023) Taurine deficiency as a driver of aging. Science 380(6649):eabn9257.
75. Huxtable R (1992) Physiological actions of taurine. Physiological reviews 72(1):101-163.
76. Cipak A, Jaganjac M, Tehlivets O, Kohlwein S D, & Zarkovic N (2008) Adaptation to oxidative stress induced by polyunsaturated fatty acids in yeast. Biochimica et Biophysica Acta (BBA)-Molecular and Cell Biology of Lipids 1781(6-7):283-287.
77. Andrisic L, et al. (2015) Transcriptional and antioxidative responses to endogenous polyunsaturated fatty acid accumulation in yeast. Molecular and cellular biochemistry 399(1-2):27-37.
78. Rojas L A, et al. (2011) Characterization of the metabolically modified heavy metal-resistant Cupriavidus metallidurans strain MSR33 generated for mercury bioremediation. PloS one 6(3):e17555.
79. Sher S & Rehman A (2019) Use of heavy metals resistant bacteria—a strategy for arsenic bioremediation. Applied microbiology and biotechnology 103(15):6007-6021.
80. Farnberger J E, et al. (2018) Biocatalytic methylation and demethylation via a shuttle catalysis concept involving corrinoid proteins. Communications Chemistry 1(1):82.
81. Frazzon J, Fick J, & Dean D (2002) Biosynthesis of iron-sulphur clusters is a complex and highly conserved process. Biochemical Society Transactions 30(4):680-685.

### Example 3—Synthetic Operon for the Production of 2-Mercaptoethane Sulfonate (Coenzyme M)

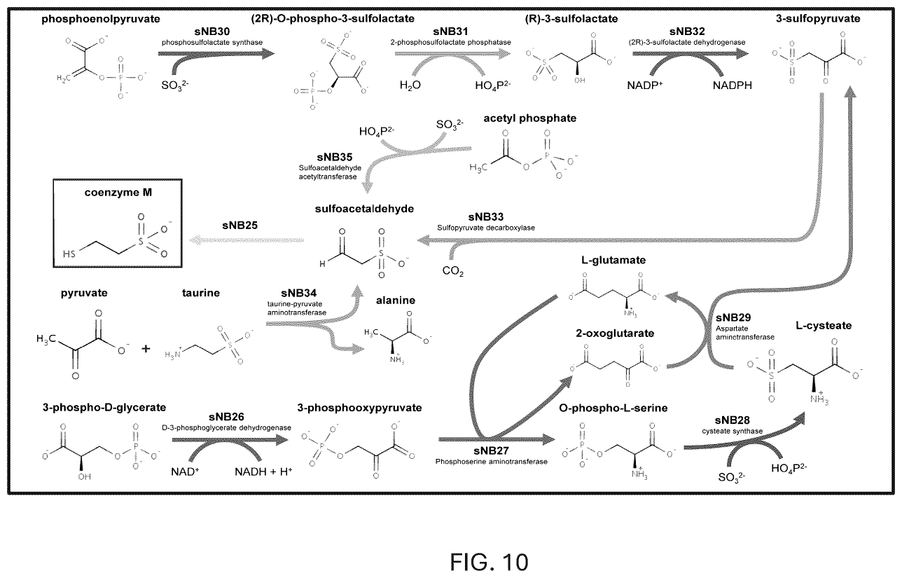

Seven synthetic pathways have been designed to allow bacteria, archaea, and eukarya (including plants) to synthesize 2-mercaptoethanesulfonate, also known as coenzyme M or commercially as MESNA (FIG. 36). In this disclosure, seven synthetic operons were designed to co-express genes in E. coli using the pET24a plasmid vector as a proof-of principle demonstration. The first four genes of Operon 1 utilize a known metabolic pathway for sulfoacetaldehyde production in methanogens with a fifth gene catalyzing the conversion of sulfoacetaldehyde into coenzyme M. Operon 1 contains genes of five enzymes from four different bacteria and archaea: phosphosulfolactate synthase from Methanocaldococcus jannaschii (sNB30), 2-phosphosulfolactate phosphatase from M. jannaschii (sNB31), (2R)-3-sulfolactate dehydrogenase from Chromohalobacter salexigens (sNB32), sulfopyruvate decarboxylase from Methanosarcina acetivorans (sNB33), and an uncharacterized enzyme from M. acetivorans (sNB25) (FIG. 8-A). This combination of enzymes converts phophoenolpyruvate to (2R)—O-phospho-3-sulfolactate by sNB30, then to (R)-3-sulfolactate by sNB31, then to 3-sulfopyruvate by sNB32, then to sulfoacetaldehyde by sNB33, and finally to coenzyme M by sNB25 (FIG. 10). Synthetic operon 2.0 principally utilizes three enzymes: cysteate synthase from Methanoculleus marisnigri (sNB28) chosen for it's high aliphatic index predicted by ExPASy ProtParam, sNB33, and sNB25 (FIG. 8-B). This pathway utilizes sNB28 to convert O-phospho-L-serine into L-cysteate which may be converted into 3-sulfopyruvate by the endogenous Escherichia coli enzyme aspartate aminotransferase (Uniprot: JW0911) and 3-sulfopyruvate will be converted to coenzyme M by both sNB33 and sNB25 as previously described (FIG. 10). Operon 2.1 (FIG. 8-C) is designed to use an aspartate aminotransferase from M. jannaschii (sNB29) which may catalyze the conversion of L-cysteate to 3-sulfopyruvate with higher efficacy than the E. coli endogenous enzyme. While Operons 2.0 and 2.1 begin with O-phospho-L-serine, the metabolic pathway being utilized can be traced backwards to 3-phospho-D-glycerate which is a high flux metabolite in plants. Operon 6 has been designed to incorporate two additional enzymes into Operon 2.1 in order to allow greater flux towards coenzyme M production. The two additional enzymes are D-3-phosphoglycerate dehydrogenase from M. jannaschii (sNB26) and phosphoserine aminotransferase from Methanosarcina barkeri (sNB27) (FIG. 8-G). sNB26 catalyzes the conversion of 3-phospho-D-glycerate to 3-phosphooxypyruvate while sNB27 catalyzes the conversion of 3-phosphooxypyruvate into O-phospho-L-serine (FIG. 10). E. coli does possess homologs of both enzymes however this may not hold true for other organisms which would potentially benefit from coenzyme M production. Operon 3 consists of two genes, the first encodes the enzyme taurine-pyruvate aminotransferase from the organism Bilophila wadsworthia (sNB34) with the second being sNB25 (FIG. 8-D). The purpose of this operon is to introduce a novel metabolic pathway where supplemental taurine and the high flux metabolite pyruvate can be converted to sulfoacetaldehyde by sNB34 and the sulfoacetaldehyde can be converted to coenzyme M by sNB25 (FIG. 10). This operon would be particularly useful in fish and animals where taurine is a more common metabolite [1]. Operon 4 converts the central metabolite acetylphosphate to sulfoacetaldehyde (FIG. 10) using sulfoacetaldehyde acetyltransferase from Castellaniella defragrans (sNB35), selected for the organism's strictly aerobic nature, in combination with sNB25 (FIG. 8-E). An operon for the production of coenzyme M using phosphoenolpyruvate (Operon 1) or acetylphosphate (Operon 4) as its initial substrate would lend itself towards utilization in any organism due to their integral roles to glycolysis and the tricarboxylic acid (TCA) cycle respectively. Operon 5 has been generated with both sNB34 and sNB33 with the hypothesis that sNB25 may not be necessary in the production of coenzyme M and that perhaps sNB33 possesses the catalytic activity necessary to produce coenzyme M from sulfoacetaldehyde.

There are several innovations that were introduced to create a functional synthetic operon. To express genes as an operon rather than as a monocistronic transcript, as is the common use for pET24a vectors, is itself an innovation. The synthetic operons contain several innovative features, including a 5′ leading sequence to the ribosome binding site generated using the RBS Calculator by De Novo DNA which was then manually trimmed to streamline the genetic element while maintaining high gene expression. Additional short ribosome binding sequences were placed in between each gene of a particular operon with the belief that these sequences would lead to higher retention of the ribosome as it processes the transcript. For the first gene in each operon an NdeI site (CATATG) was added prior to the gene as part of spacer sequence between the Shine Dalgarno sequence with a built-in start codon (ATG). This is intended not only to conserve space between the ribosome binding sequence (TAAGGAGGT) and the RNA sequence which would be transcribed but also to allow modularity for the insertion of other genes if necessary. The 3′ end of each gene was changed to utilize opal and ochre stop codons (TGATAA) as it has been observed that ribosomes recognize an opal codon as a soft stop and an ochre codon as a hard stop and experience difficulty terminating translation with the presence of only one stop codon [2-4]. We hypothesized that dual ochre codons would initiate an undesired disconnection of the ribosome from the transcript. It was also hypothesized that the two 3′ adenines of an ochre codon would lead to the formation of the Shine Dalgarno sequence (AAGGAGG) being placed in between the two genes to promote ribosome retention once the translation of the prior gene is completed. Between the genes, restriction enzyme (RE) sites were placed in the space between the Shine Dalgarno sequence in the following order: BamHI (GGATCC), SpeI (ACTAGT), XhoI (CTCGAG), Bsp1407I (TGTACA), and Sad (GAGCTC) with the last site of any operon being HindIII (AAGCTT). These RE sites were chosen for their ability to create overhangs, to be utilized as a start codon (i.e. NdeI), to be worked into Shine Dalgarno sequences (i.e. first three bases of BamHI, first base of SpeI, Bsp14071, and SacI), and to be integrated into the final stop codon of an operon (i.e. HindIII). We hypothesized that this selection of endonuclease sites would lend themselves to the conservation of space between genes while ensuring that the desired modularity would not negatively impact ribosome retention on the mRNA as the ribosome moves between the two genes of the constructed operon.

The protein sequences were obtained through Uniprot and the corresponding DNA sequences were generated for optimal expression in E. coli. Vector NTI software (Invitrogen) was then used to identify recognition sites of the restriction enzymes being utilized. These sites were eliminated by manually altering the codons through replacing nucleic bases with the intention of maintaining the amino acid sequence, using high abundance codons to maximize translation efficiency, and attempting to minimize the possibility of stable hairpin loop formation by nascent transcripts. Additionally, a 4×Gly 6×His tag was added to the N terminus of each protein in order to facilitate possible purification and identification; these tags were designed to have different coding sequences with the express purpose of avoiding substantial homology which would lead to issues with operon construction. A summary of the genes used and the design features incorporated to allow synthetic operon construction is found in Table 9. As a result of these efforts, the nucleic acid sequences are novel synthetic constructions that would not be predicted using E. coli gene expression optimization algorithms.

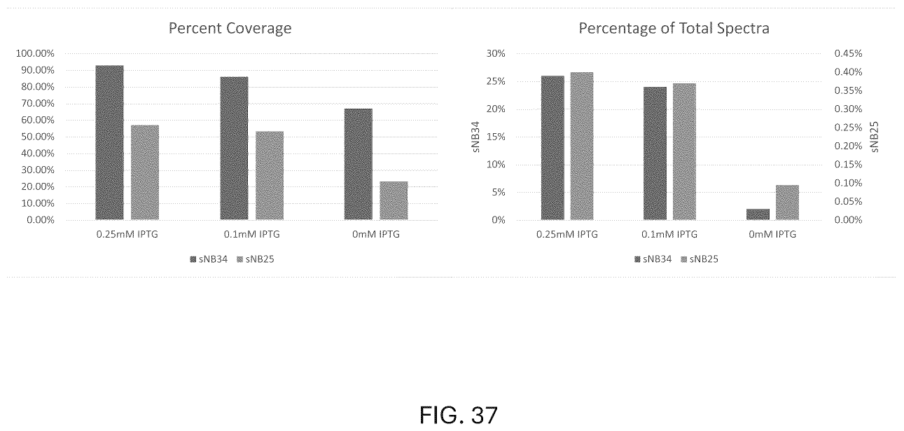

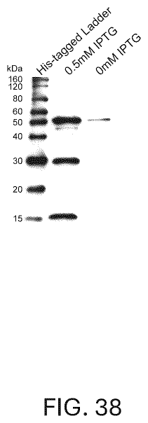

The utilization of an operon for the expression of the final enzyme (MA3299) of our pathways was critical towards achieving enzymatic activity and the production of Coenzyme M. Direct protein overexpression of MA3299 was observed to be impossible, a result which aligns with the experiences of other researchers who have tried overexpressing archaeal iron-sulfur (Fe—S) cluster enzymes [5]. BL21 E. coli would quickly silence the promoters involved in overexpression to prevent the production of MA3299 and no protein was observed through Western Blotting. Pairing MA3299 into an operon with Q9APM5 (pCH003 in FIG. 36) with MA3299 being the second protein would result in a lower amount of MA3299 expressed while allowing the E. coli to produce another enzyme important for establishing a metabolic pathway to Coenzyme M. However, there were several more nonobvious changes necessary to express and detect the enzymatic activity of MA3299. Growth in rich medias, e.g. the standard recommended Lysogeny Broth and Super Optimal Broth, showed no production of MA3299 under expression within the operon. The switch to a Defined Media for a higher doubling time proved successful in the expression of both proteins as could be observed through Western Blot analysis (FIG. 37) and LC-MS/MS peptide analysis (FIG. 38)

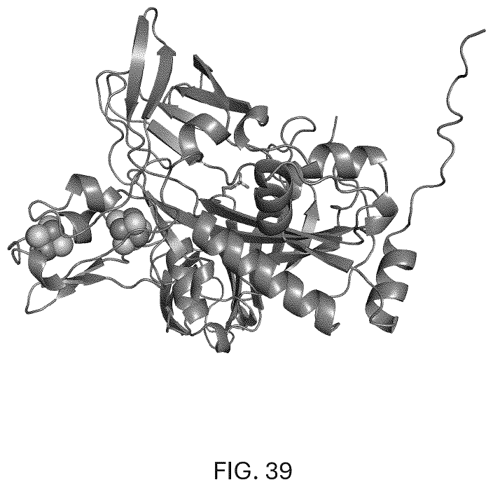

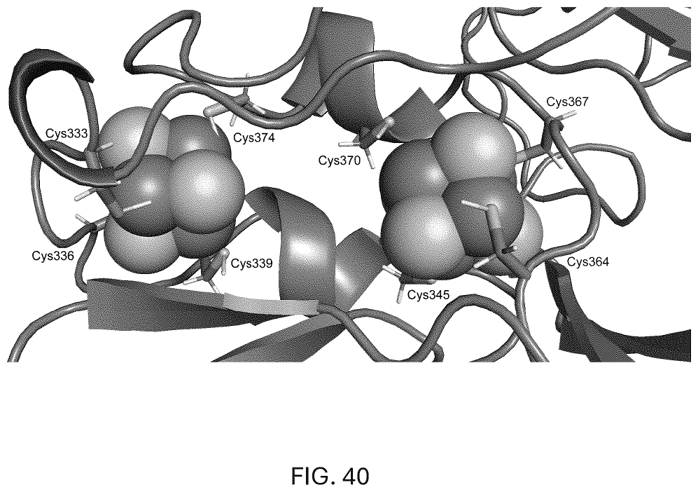

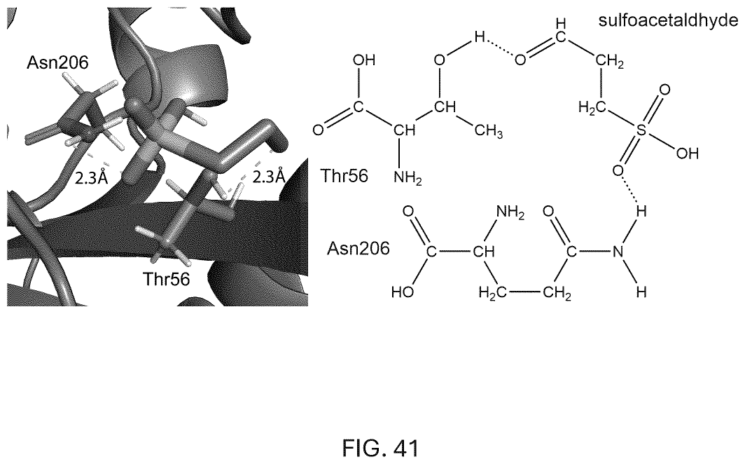

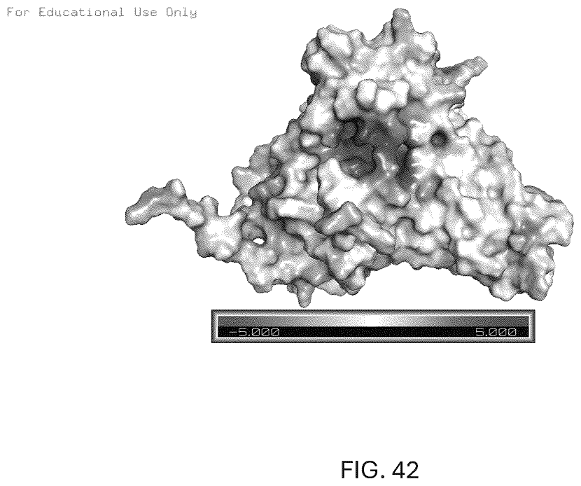

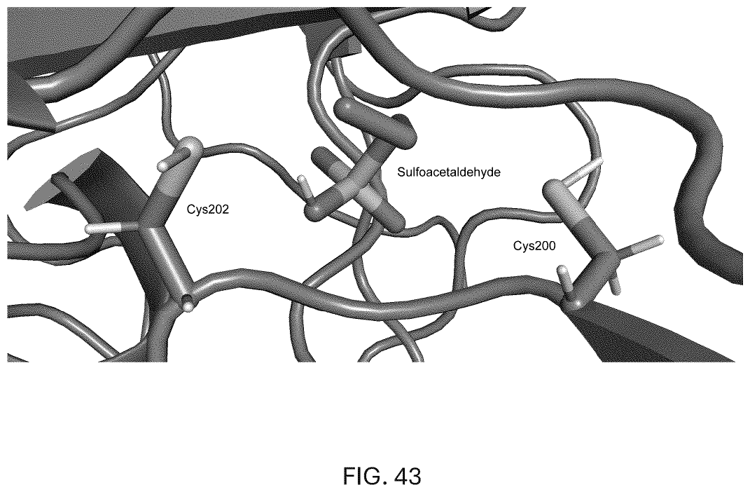

We then modeled MA3299 structure to determine if our difficulty in expressing MA3299 could have been caused by the need for one or more Fe—S clusters. After generating protein structures of MA3299 with Alphafold [6], the best fit structure was used with Autodock [7] to fit 4Fe-4S clusters and sulfoacetaldehyde into the protein structure (FIG. 39). Two different locations were found with a high affinity for 4Fe-4S Clusters: the first location utilizes Cys333, Cys336, Cys339, and Cys374 while the second location utilizes Cys345, Cys364, Cys367, and Cys370 (FIG. 40). Sulfoacetaldehyde has the potential to hydrogen bond with Thr56 and Asn206 within the center of MA3299 (FIG. 41) through a hydrophilic pocket lined with basic residues (FIG. 42). These basic residues allow potential electron donors to reach lower standard redox potentials and assist with electron transfer. This area could also assist with sulfur transfer between a sulfur donor and one of the two cysteines, Cys200 or Cys202 which sit within the catalytic site (FIG. 43). To further improve expression and enzymatic activity of MA3299, the plasmid pCH003 was co-transformed with pDB1282 into BL21 E. coli to allow for expression of the isc operon under an arabinose inducible promoter. pDB1282 is typically used with the expression of Fe—S cluster proteins in order to provide the overexpressed proteins with the Fe—S clusters they require for enzymatic activity [8].

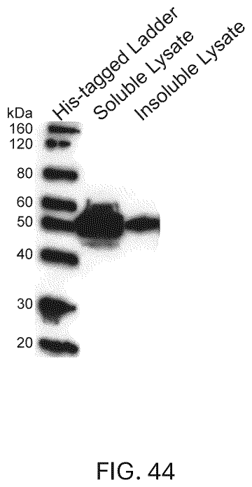

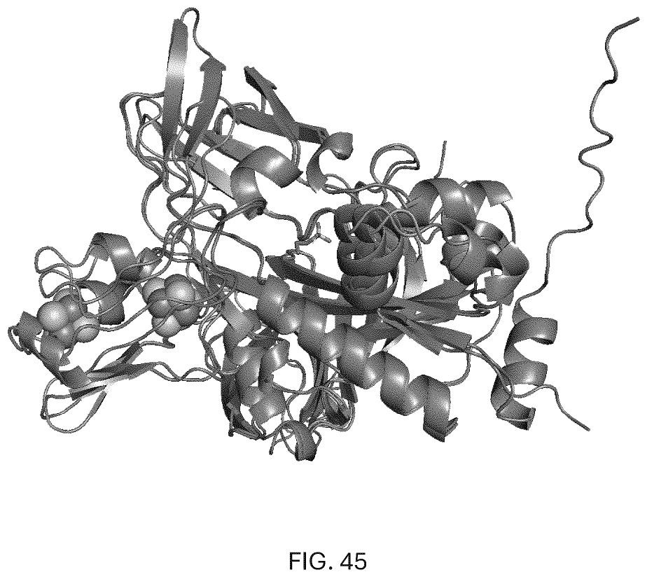

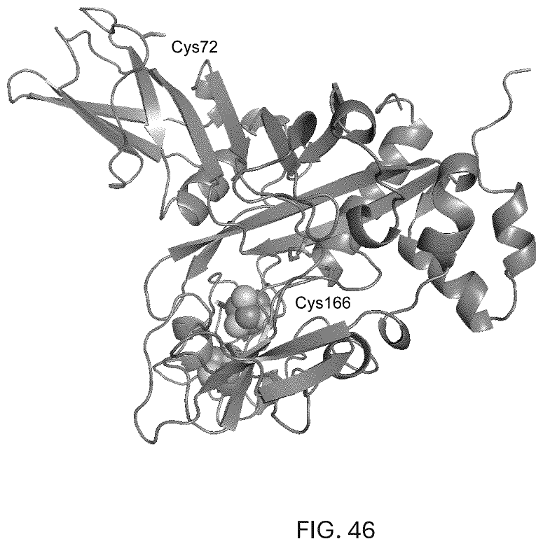

Following the recommended Fe—S protein expression protocol, expression of MA3299 was once again mitigated by the expression of pDB1282 in conjunction with supplemental 1 mM Iron (II) Sulfate, 1 mM Ferric Ammonium Sulfate, and 2 mM Cysteine. The suppression of expression was alleviated by withholding the supplements and as a result we were able to see the highest expression of MA3299 in conjunction with Q9APM5 observed thus far. These results indicate that MA3299, as a holoenzyme, is unstable in E. coli when overexpressed while the expression of MA3299, as an apoenzyme, remains a viable avenue for overproduction of the enzyme (FIG. 44). The function of MA3299 and similar Fe—S proteins have remained a mystery as a knockout of Mmp1603 in Methanococcus maripaludis did not show reliance on the absence or supplementation of CoM addition to the medium [5]. The enzymatic activity of a similar Fe—S protein, MJ1681 from Methanocaldococcus jannashii, is hypothesized to metabolize the product of ComDE, sulfoacetaldehyde into coenzyme M through a pathway proposed by Robert H. White [5]. While the two enzymes, MA3299 and MJ1681, only possess 41.34% similarity with one another, their overall Alphafold models align with each other enough to superimpose their peptide backbones (FIG. 45). White points towards two cysteine residues within MJ1681 that he believes mediate the sulfur transfer to produce CoM, Cys72 and Cys166, however an Alphafold construct of MJ1681 reveals that Cys72 in located on the outside of the enzyme, too distant from Cys166 to mediate this reaction (FIG. 46). Using the Alphafold construct of MJ1681, the only other cysteine within proximity with Cys166 to function within the mechanism proposed by White is Cys36, however, this cysteine is located on the opposite side of the active site. While there are some similarities in the position of what is proposed to be a critical cysteine residue, there are significant differences with other key residues, suggesting MA3299 and MJ1681 could have different enzyme mechanisms. Specifically, the placement of Cys202 in MA3299 and Cys166 in MJ1681 are at the same location however the two cysteines necessary for the catalytic mechanism proposed by White are too close in sequence to form a disulfide bond with one another, indicating that the dual cysteine mechanism for catalyzing CoM in the manner White has proposed is not possible in MA3299 or MJ1681 (FIG. 43). White overexpressed MJ1681 in E. coli however he was unable to detect MJ1681 in soluble fractions of cell lysate, suggesting the protein was mostly misfolded. White was only able to confirm CoM synthesis after incubating the overexpression strain with 17 mM sulfoacetaldehyde and concentrating a 10 mL culture 300× to 600 μL. It should be noted that the extent to which White was able to produce CoM is unknown and the LC-MS measurements are not provided in the manuscript.

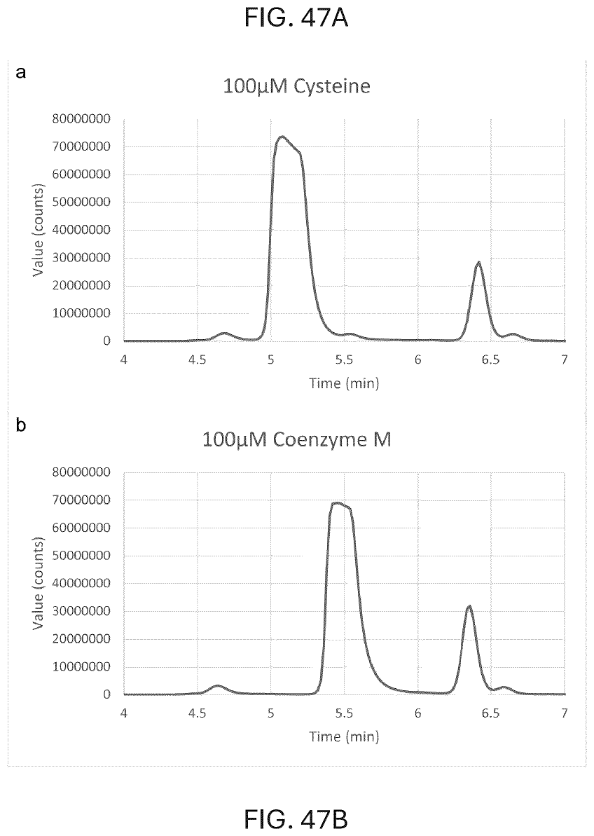

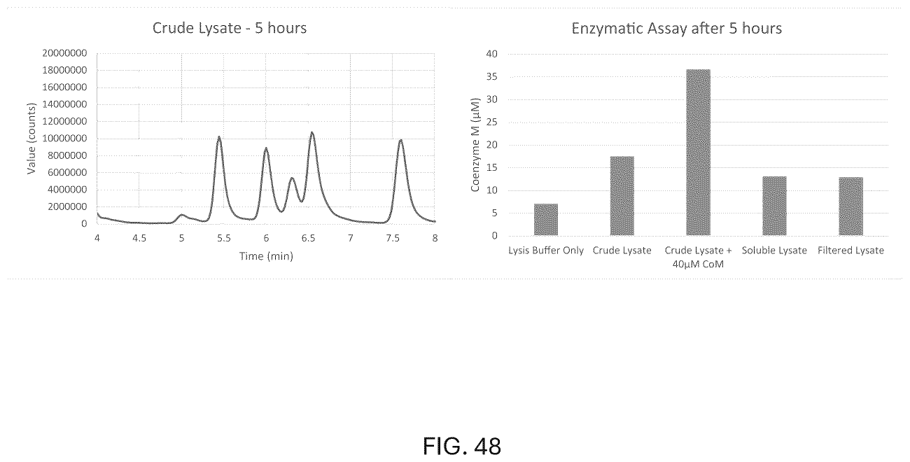

We were able to confirm function of Q9APM5 and MA3299 activity through co-expression of pCH003 with pDB1282 in 500 mL Defined Media in non-baffled flasks with kanamycin and ampicillin grown at 37° C. and 250 rpm. 2% Arabinose was given at OD600=0.5 and 0.1 mM IPTG was given 15 minutes after then incubated at 18° C. and 250 rpm for 18 hours. The final OD600 for the culture was 5.4 and the culture was pelleted in a centrifuge at 8,000×g and 4° C. for 10 minutes. 1.56 g of this pellet was transferred into an anaerobic chamber then solubilized in 10 mL of anaerobic Lysis Buffer. This cell suspension was then lysed by sonication with 40% amplitude and 30 s on/off cycles for 10 minutes. Aliquots were reserved in 2 mL glass autosampler vials and kept on ice while the rest of the lysate was removed from the chamber in an anaerobic Oakridge bottle and centrifuged at 10,000×g and 4° C. for 10 minutes then brought back into the chamber. More aliquots were taken then the soluble fraction was filtered using a 0.2 μm SFCA/PF filter. 1 mL of each of these samples were then given 10 mM Iron (II) Sulfate Heptahydrate and 10 mM Sodium Sulfide Nonahydrate and incubated at room temperature for 20 minutes to allow the overexpressed isc proteins to generate additional Fe—S Clusters and allow them to be loaded into MA3299 [9]. These samples were placed in a 37° C. heat block and given 10 mM Sodium Pyruvate, 10 mM Taurine, and 50 mM Potassium Borohydride to initiate the enzyme assay (This study). 250 μL samples were taken at 1 minute, 10 minutes, 30 minutes, and 5 hours then immediately derivatized using monobromobimane following the procedure outlined by Rijstenbil and Wijnholds [10]. These samples were stored at 4° C. until analyzed using a Supelcosil LC-18 column with a Fluorescence detector recording 470 nm emissions with 380 nm excitation. The chromatographic separation conditions follow those set by Minocha et al. with the retention times of Cysteine at 5 minutes and Coenzyme M at 5.5 minutes (FIG. 47) [11]. Within our samples taken at 5 hours, a Coenzyme M peak is registered in the Lysis Buffer only sample with a concentration of 7.06 μM representing an autocatalysis of the compound. The crude lysate sample registered a Coenzyme M peak with a concentration of 17.49 μM, demonstrating a 148% increase in the production of Coenzyme M. A sample of the same crude lysate supplemented with 40 μM Coenzyme M shows a peak at the same retention time demonstrating that this peak contains Coenzyme M (FIG. 48). The soluble protein lysate samples which had been removed from the chamber showed Coenzyme M peaks corresponding to 13.07 μM (unfiltered lysate) and 12.88 μM (filtered lysate), aligning with the autocatalysis observed in the Lysis Buffer only sample. Removing the cell lysate from the anaerobic chamber for centrifugation reduced the enzymatic activity of MA3299 by ˜60%. Catalysis was not observed using Q9APM5 (sNB34) alone. Considering that we began with a 500 mL culture which yielded a 4.4 g pellet and only 1.56 g of that pellet was solubilized in 10 mL Lysis Buffer, when compared to the assay White performed using concentrated sulfoacetaldehyde, our assay required only a 17.7× concentration of cell lysate as opposed to the 300× White needed to register enzyme-derived production of CoM.

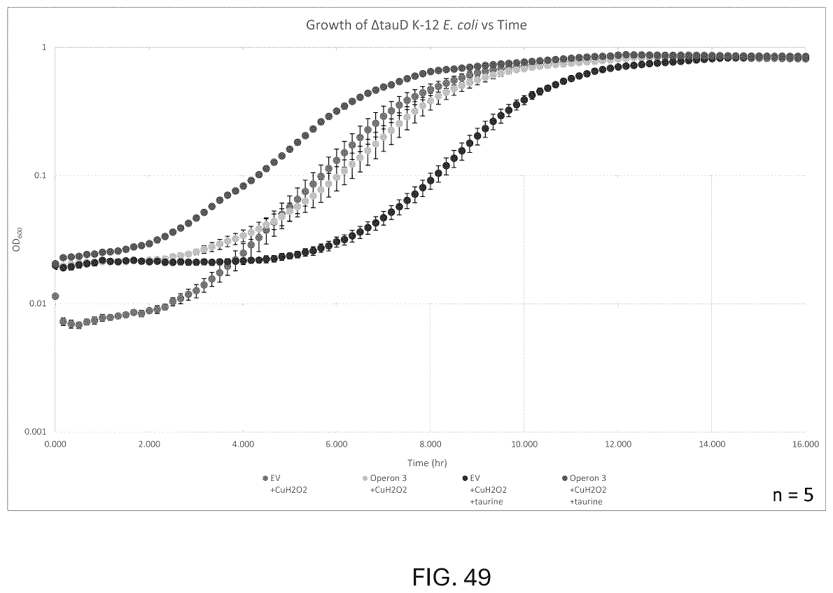

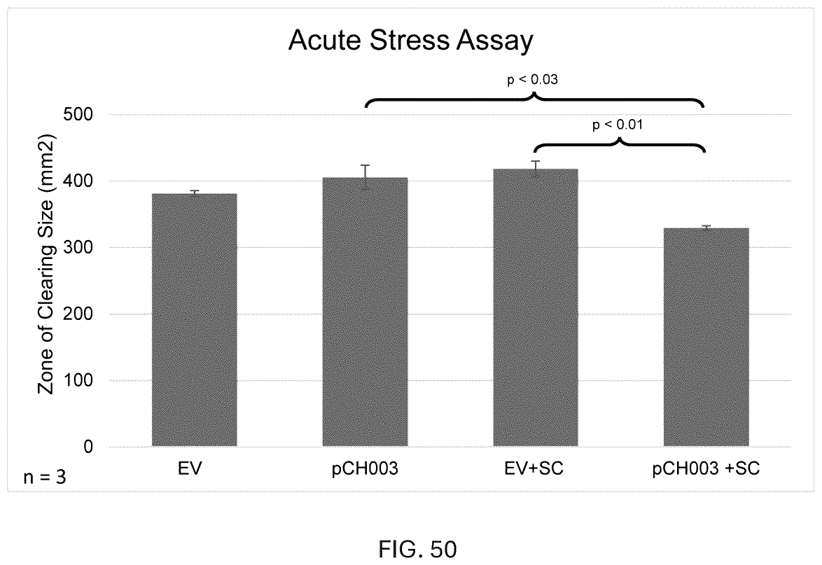

As CoM is a thiol with proposed antioxidant qualities, we tested the ability of operon 3 to protect E. coli from oxidative stress. ΔtauD K-12 E. coli were made kanamycin sensitive through the use of the pCP20 plasmid [12] and given a T7 Polymerase using a λDE3 Lysogenization Kit (Novagen). This strain was transformed with pCH003 and expression of operon 3 in the presence of taurine was shown to accelerate the recovery of ΔtauD E. coli when oxidatively stressed by exposure to 180 μM cumene hydroperoxide (FIG. 49). Expression of operon 3 was induced for 2 hours using 100 μM IPTG and cultures were incubated with 100 μM taurine for 70 minutes before being used as inoculum in a 96 well plate with cumene hydroperoxide. Optical density (OD) measurements at 600 nm were taken every 6 minutes for 17.5 hours using a Tecan Microplate Reader. A Kirby-Bauer assay was developed which examined the zone of clearing around sterile absorption disks dosed with 5 μL 2.45M hydrogen peroxide. These assays showed resistance of operon 3 expressing K-12 E. coli which had been grown with 0.1 mM taurine allowing the E. coli to grow closer in proximity to the disk represented by a decrease in the zone of clearing (FIG. 1750

Our study shows that we were successful in generating a synthetic operon to produce a non-native antioxidant, coenzyme M, in E. coli, and that expression of the operon resulted in increased resistance to oxidative stress in both chronic and acute stress tests.

TABLE 9
Genes synthesized

| | Modified | | | |
|---|---|---|---|---|
| | from | String | | |
| Enzyme Name | Accession # | ID¹ | Operon | Types of Modification² |
| uncharacterized enzyme | Q8TKU5 | sNB25 | 1, 2.0, 2.1, | RE site removal, hairpin elimination, |
| | | | 3, 4, 6 | His tag |
| D-3-phosphoglycerate | Q58424 | sNB26 | 6 | RE site removal, hairpin elimination, |
| dehydrogenase | | | | RBS insertion, His tag |
| phosphoserine | P52878 | sNB27 | 6 | RE site removal, hairpin elimination, |
| aminotransferase | | | | RBS insertion, His tag |
| cysteate synthase | A3CRP6 | sNB28 | 2.0, 2.1, | RE site removal, hairpin elimination, |
| | | | 6 | RBS w/leader insertion, His tag |
| aspartate | Q60317 | sNB29 | 2.1, 6 | RE site removal, hairpin elimination, |
| aminotransferase | | | | RBS insertion, His tag |
| phosphosulfolactate | Q57703 | sNB30 | 1 | RE site removal, hairpin elimination, |
| synthase | | | | RBS w/leader insertion, His tag |
| 2-phosphosulfolactate | Q58540 | sNB31 | 1 | RE site removal, hairpin elimination, |
| phosphatase | | | | RBS insertion, His tag |
| (2R)-3-sulfolactate | Q1QWN5 | sNB32 | 1 | RE site removal, hairpin elimination, |
| dehydrogenase | | | | RBS insertion, His tag |
| sulfopyruvate | Q8TKU6 | sNB33 | 1, 2.0, 2.1, | RE site removal, hairpin elimination, |
| decarboxylase | | | 5, 6 | His tag |
| taurine-pyruvate | Q9APM5 | sNB34 | 3, 5 | RE site removal, hairpin elimination, |
| aminotransferase | | | | His tag |
| sulfoacetaldehyde | Q84H44 | sNB35 | 4 | RE site removal, hairpin elimination, |
| acetyltransferase | | | | RBS w/leader insertion, His tag |

TABLE 10
Disclosed operons and exemplary descriptions

| Operon # | Plasmid | Features |
|---|---|---|
| 1 | pCH008 | Utilizes known metabolic pathway in |
| | | methanogens |
| 2 | pCH009 | Utilizes an endogenous E. coli enzyme |
| 2.1 | pCH010 | Alteration to #2 for higher efficiency enzyme |
| | | compared to endogenous E. coli enzyme → more |
| | | CoM production |
| 3 | pCH003 | Novel pathway for production where taurine and |
| | | high flux pyruvate can be converted to CoM |
| | | → Useful in fish & animals where taurine is a |
| | | more common metabolite |
| 4 | pCH004 | Utilizes initial substrates that lend themselves |
| | | towards utilization in any organism due to integral |
| | | roles in glycolysis and TCA cycle |
| 5 | pCH005 | Experimental operon that suggest sNB25 is not |
| | | necessary for CoM production |
| 6 | pCH011 | = Operon 2.1 + two additional enzymes that other |
| | | organisms may not have for greater CoM |
| | | production → Can be traced back to 3-phospho- |
| | | D-glycerate which is a high flux plant metabolite |

Sequences

| SEQ ID NO | Description |
|---|---|
| 1 | sNB25, ComF, MA3299, without 4xG and 6xH tags |
| 2 | sNB25, ComF, MA3299, with 4xG and 6xH tags |
| 3 | sNB34, Tpa, Q9APM5 without 4xG and 6xH tags |
| 4 | sNB34, Tpa, Q9APM5 with 4xG and 6xH tags |
| 5 | sNB35, sulfoacetaldehyde acetyltransferase |
| 6 | sNB33, sulfopyruvate decarboxylase |
| 7 | sNB26, D-3-phosphoglycerate dehydrogenase |
| 8 | sNB27, phosphoserine aminotransferase |
| 9 | sNB28, cysteate synthase |
| 10 | sNB29, aspartate aminotransferase |
| 11 | sNB30, phosphosulfolactate synthase |
| 12 | sNB31, 2-phosphosulfolactate phosphatase |
| 13 | sNB32, (2R)-3-sulfolactate dehydrogenase |
| 14 | sNB35, sulfoacetaldehyde acetyltransferase his tag |
| 15 | sNB33, sulfopyruvate decarboxylase his tag |
| 16 | sNB26, D-3-phosphoglycerate dehydrogenase his tag |
| 17 | sNB27, phosphoserine aminotransferase his tag |
| 18 | sNB28, cysteate synthase his tag |
| 19 | sNB29, aspartate aminotransferase his tag |
| 20 | sNB30, phosphosulfolactate synthase his tag |
| 21 | sNB31, 2-phosphosulfolactate phosphatase his tag |
| 22 | sNB32, (2R)-3-sulfolactate dehydrogenase his tag |
| 23 | sNB25 DNA, E. coli optimized |
| 24 | sNB34 DNA, E. coli optimized |
| 25 | sNB35, sulfoacetaldehyde acetyltransferase |
| 26 | sNB33, sulfopyruvate decarboxylase |
| 27 | sNB26, D-3-phosphoglycerate dehydrogenase |
| 28 | sNB27, phosphoserine aminotransferase |
| 29 | sNB28, cysteate synthase |
| 30 | sNB29, aspartate aminotransferase |
| 31 | sNB30, phosphosulfolactate synthase |
| 32 | sNB31, 2-phosphosulfolactate phosphatase |
| 33 | sNB32, (2R)-3-sulfolactate dehydrogenase |
| 34 | pCH003 plasmid |
| 35 | pCH019 plasmid |
| 36 | pCH020 plasmid |
| 37 | CMV promoter |
| 38 | EF1a promoter |
| 39 | CAG promoter |
| 40 | PGK promoter |
| 41 | TRE promoter |
| 42 | U6 promoter |
| 43 | UAS promoter |
| 44 | oNB924 |
| 45 | oNB925 |
| 46 | oNB926 |
| 47 | oNB927 |
| 48 | oNB928 |
| 49 | oNB929 |
| 50 | oNB52 |
| 51 | oNB53 |
| 52 | oNB54 |
| 53 | oNB55 |
| 54 | oNB95 |
| 55 | oNB96 |
| 56 | oNB98 |
| 57 | oNB99 |
| 58 | oNB103 |
| 59 | oNB104 |
| 60 | oNB121 |
| 61 | oNB122 |
| 62 | oNB123 |
| 63 | oNB 124 |
| 64 | Peptide sequence on p. 61 of spec |
| 65 | Lac operator |
| 66 | Full sequence operon 1 |
| 67 | Full sequence operon 2 |
| 68 | Full sequence operon 2.1 |
| 69 | Full sequence operon 3 |
| 70 | Full sequence operon 4 |
| 71 | Full sequence operon 5 |
| 72 | Full sequence operon 6 |
| 73 | 5′ leading sequence for operon 1 |
| 74 | 5′ leading sequence for operon 2 |
| 75 | 5′ leading sequence for operon 3 |
| 76 | 5′ leading sequence for operon 4 |
| 77 | 5′ leading sequence for operon 5 |
| 78 | M. mazei Go1 |
| 79 | >M. barkeri CM1 |
| 80 | >M. evestigatum Z7303 |
| 81 | >M. mahii DSM 5219 |
| 82 | >M. burtonii DSM 6242 |
| 83 | >M. arvoryzae MRE50 |
| 84 | >M. palustris E19c |
| 85 | >M. marisnigri JR1 |
| 86 | >M. labreanum Z |
| 87 | >M. petrolearia DSM 11571 |
| 88 | >M. hungatei JF1 |
| 89 | >M. thermautotrophicus str. Delta H |
| 90 | >M. marburgensis str. Marburg |
| 91 | >M. fervidus DSM 2088 |
| 92 | M. smithii ATCC 35061 |
| 93 | >M. formicicum DSM 3637 |
| 94 | >M. jannaschii DSM 2661 |
| 95 | >M. voltae PS |
| 96 | >M. maripaludis S2 |
| 97 | >M. formicicus McS70 |
| 98 | >M. okinawensis IH1 |
| 99 | >M. aeolicus Nankai3 |
| 100 | >M. kandleri AV19 |

We claim:

1. A polynucleotide comprising a sequence encoding a coenzyme M synthase (ComF) operably linked to at least one heterologous regulatory element.

2. The polynucleotide of claim 1, wherein the ComF is Methanosarcina acetivorans ComF.

3. The polynucleotide of claim 2, wherein the ComF comprises SEQ ID NO: 1 or a sequence with at least 90% identity to SEQ ID NO: 1.

4. The polynucleotide of claim 1, wherein the polynucleotide further comprises a sequence encoding a sulfoacetaldehyde-producing enzyme.

5. The polynucleotide of claim 4, wherein the sulfoacetaldehyde-producing enzyme comprises a taurine-pyruvate aminotransferase, a sulfoacetaldehyde acetyl transferase, or a sulfopyruvate decarboxylase.

6. The polynucleotide of claim 5, wherein the taurine-pyruvate aminotransferase comprises SEQ ID NO: 3 or a sequence with at least 90% identity to SEQ ID NO: 3.

7. The polynucleotide of claim 5, wherein the sulfoacetaldehyde acetyl transferase comprises SEQ ID NO: 5 or a sequence with at least 80% identity to SEQ ID NO: 5.

8. The polynucleotide of claim 5, wherein the sulfopyruvate decarboxylase comprises SEQ ID NO: 6 or a sequence with at least 80% identity to SEQ ID NO: 6.

9. The polynucleotide of claim 8, wherein the polynucleotide further comprises at least one sequence encoding a D-3-phosphoglycerate dehydrogenase, a phosphoserine aminotransferase, a cysteate synthase, an aspartate aminotransferase, a phosphosulfolactate synthase, a 2-phosphosulfolactate phosphatase, a (2R)-3-sulfolactate dehydrogenase, or a taurine-pyruvate aminotransferase.

10. The polynucleotide of claim 8, wherein the polynucleotide further comprises sequences encoding a phosphosulfolactate synthase, a 2-phosphosulfolactate phosphatase, and a (2R)-3-sulfolactate dehydrogenase.

11. The polynucleotide of claim 8, wherein the polynucleotide further comprises a sequence encoding a cysteate synthase.

12. The polynucleotide of claim 11, wherein the polynucleotide further comprises a sequence encoding an aspartate aminotransferase.

13. The polynucleotide of claim 8, wherein the polynucleotide further comprises sequences encoding a cysteate synthase, an aspartate aminotransferase, a D-3-phosphoglycerate dehydrogenase, and a phosphoserine aminotransferase.

14. The polynucleotide of claim 9, wherein
(i) the D-3-phosphoglycerate dehydrogenase comprises SEQ ID NO: 7, or a sequence with at least 90% identity to SEQ ID NO: 7;
(ii) the phosphoserine aminotransferase comprises SEQ ID NO: 8, or a sequence with at least 90% identity to SEQ ID NO: 8;
(iii) the cysteate synthase comprises SEQ ID NO: 9, or a sequence with at least 90% identity to SEQ ID NO: 9;
(iv) the aspartate aminotransferase comprises SEQ ID NO: 10, or a sequence with at least 90% identity to SEQ ID NO: 10;
(v) the phosphosulfolactate synthase comprises SEQ ID NO: 11, or a sequence with at least 90% identity to SEQ ID NO: 11;
(vi) the 2-phosphosulfolactate phosphatase comprises SEQ ID NO: 12, or a sequence with at least 90% identity to SEQ ID NO: 12;
(vii) the (2R)-3-sulfolactate dehydrogenase comprises SEQ ID NO: 13, or a sequence with at least 90% identity to SEQ ID NO: 13, or
(viii) the taurine-pyruvate aminotransferase comprises SEQ ID NO: 3, or a sequence with at least 90% identity to SEQ ID NO: 3.

15. A cell comprising the polynucleotide of claim 1.

16. A method comprising introducing the polynucleotide of claim 1 into a cell.

17. A method of increasing aerial tissue growth in a plant, the method comprising expressing the polynucleotide of claim 1 in cells of the plant and growing the plant.

18. A method of increasing dry weight of a plant comprising expressing the polynucleotide of claim 1 in cells of the plant and growing the plant.

19. A method of producing coenzyme M (CoM), the method comprising culturing cells comprising the polynucleotide of claim 1 and harvesting the CoM from the cultured cells.

20. A recombinant archaeal cell comprising a polynucleotide encoding a cysteate synthase or a sequence encoding a sulfopyruvate decarboxylase, wherein the sequence encoding a cysteate synthase or the sulfopyruvate decarboxylase is operably linked to a heterologous regulatory element.


SEQUENCE LISTING (100 sequences)

SEQ ID NO: 1 - protein, 438 aa, Methanosarcina acetivorans
MNGALDNTEKPRTIQEIQVKIDAGEAVVLTAEEIGAKVRAGGEIRLEDIDVVTTATRGIMSGTYAVFSFKVSEPDSFVKASKVLLNGVPAIVGPCPNERLGVLDLIVLGTAHSETDSRYGGGHLFRDMVEGKSITVDVTTSVGDFFSVETRLSEMPFARLYATRHAFKNYRAFVNPGKESIKTIFHALPFEGEFRELTFCGCGELNPIQNDPKLETIGIGTRVLINGAEGFVTGQGTRSAPDNPNLTGFADLHDMTPEYMGGFVTSAGPEIVNTWAVPIPVLSREMLENILQLDSQIPLKLVDLAGRLPLCEITYGDVWDNTDLSVKYEPEKCIDCKICCVAEACPMGAVSKGENGAVHNPELCFNCGLCISRCKGEAFSAKLGTVSCAVGGCFRDIKVTLRQSDRARAVKAAEELKAQVLSGKFRLKEPVEKISWRE

SEQ ID NO: 2 - protein, 448 aa, synthetic construct
MNGALDNTEKPRTIQEIQVKIDAGEAVVLTAEEIGAKVRAGGEIRLEDIDVVTTATRGIMSGTYAVFSFKVSEPDSFVKASKVLLNGVPAIVGPCPNERLGVLDLIVLGTAHSETDSRYGGGHLFRDMVEGKSITVDVTTSVGDFFSVETRLSEMPFARLYATRHAFKNYRAFVNPGKESIKTIFHALPFEGEFRELTFCGCGELNPIQNDPKLETIGIGTRVLINGAEGFVTGQGTRSAPDNPNLTGFADLHDMTPEYMGGFVTSAGPEIVNTWAVPIPVLSREMLENILQLDSQIPLKLVDLAGRLPLCEITYGDVWDNTDLSVKYEPEKCIDCKICCVAEACPMGAVSKGENGAVHNPELCFNCGLCISRCKGEAFSAKLGTVSCAVGGCFRDIKVTLRQSDRARAVKAAEELKAQVLSGKFRLKEPVEKISWREGGGGHHHHHH

SEQ ID NO: 3 - protein, 456 aa, Bilophila wadsworthia
MTYDKAELVALDKKYVWHHLTQHKNFEPAIYVKGEGMRITDIDGKTYLDAVSGGVWTVNVGYGRKEIVDAVAKQMMEMCYFANGIGNVPTIKFSEKLISKMPGMSRVYLSNSGSEANEKAFKIVRQIGQLKHGGKKTGILYRARDYHGTTIGTLSACGQFERKVQYGPFAPGFYEFPDCDVYRSKFGDCADLGVKMAKQLEEVILTVGPDELGAVIVEPMTAGGGILVPPAGYYETIREICDKYELLLIIDEVVCGLGRTGKWFGYQHFNVQPDIVTMAKGVASGYAPISCTVTTEKVFQDFVNDPADTDAYFRDISTFGGCTSGPAAALANIEIIERENLLENCTKMGDRLLEGLKGLMAKHPIIGDVRGKGLFAGIEIVKDRATKEPIAEAVANAMVGAAKQAGVLIGKTSRSFREFNNTLTLCPALIATEADIDEIVAGIDKAFTTVEQKFGL

SEQ ID NO: 4 - protein, 466 aa, synthetic construct
MTYDKAELVALDKKYVWHHLTQHKNFEPAIYVKGEGMRITDIDGKTYLDAVSGGVWTVNVGYGRKEIVDAVAKQMMEMCYFANGIGNVPTIKFSEKLISKMPGMSRVYLSNSGSEANEKAFKIVRQIGQLKHGGKKTGILYRARDYHGTTIGTLSACGQFERKVQYGPFAPGFYEFPDCDVYRSKFGDCADLGVKMAKQLEEVILTVGPDELGAVIVEPMTAGGGILVPPAGYYETIREICDKYELLLIIDEVVCGLGRTGKWFGYQHFNVQPDIVTMAKGVASGYAPISCTVTTEKVFQDFVNDPADTDAYFRDISTFGGCTSGPAAALANIEIIERENLLENCTKMGDRLLEGLKGLMAKHPIIGDVRGKGLFAGIEIVKDRATKEPIAEAVANAMVGAAKQAGVLIGKTSRSFREFNNTLTLCPALIATEADIDEIVAGIDKAFTTVEQKFGLGGGGHHHHHH

SEQ ID NO: 5 - protein, 598 aa, Castellaniella denitrificans
MANDTRQVVQGVQEMTPSEAFVETMVANGVTEIFGIMGSAFMDAMDIFAPAGIKLIPVVHEQGAAHMADGFARVSGRTGVVIGQNGPGISNCVTAIAAAYWAHTPVVIVTPEAGTTGIGLGGFQEARQLPMFQEFTKYQGHVTHPARMAEYTARCFARARDEMGPAQLNIPRDYFYGKIKCEIPLPQPLDRGPGGAQSLDAAARLLAEAKFPVIISGGGVVMGDAVEECKALAERLGAPVVNSYLHNDSFPASHPLWCGPLGYQGSKAAMKLLADADVVLALGTRLGPFGTLPQHGLDYWPKNARIIQVDADSKMLGLVKKITVGVCGDAKASAAEISRRIDGMKLACDANKAERAARIQAEKDAWEQELTDWTHERDPFSLDMIEEQSKEEGNWLHPRQVLRELEKAMPEDVMVSTDIGNINSVANSYLRFEKPRSFFAAMSWGNCGYAFPTIIGAKVAAPHRPAVSYAGDGAWGMSMSEIMTCVRHDIPVTAVVFHNRQWGAEKKNQVDFYNRRFVAGELESESFAGIARAMGAEGVVVDRIEDVGPALKKAIDAQMNDRKTTVIEIMCTRELGDPFRRDALSKPVRLLEKYRDYT

SEQ ID NO: 6 - protein, 387 aa, Methanosarcina acetivorans
MYVVNPEEKVIEIMKQTGIDLAATLPCDRIKNLLPLVSENFPEIKLTREENGVGICAGIYLAGGKPMMLIQSTGLGNMINALESLNVTCKIPLPILASWRGVYKEGIEAQVPLGAHLPSILEGAGLTYTIIGETEKLPLLENVILDAFENSRPHIALVSPKVWEASECCAWQAAGMPIKPEIMERTCRFSLTSGTLKPFMLRNDAICTLASELDDEITVTNLGVPCKELYACRDRELNFYMFGSMGLVSSIGLGLALRSEKTVITFDGDGSLLMNPNALLEIAKEAPKNLIIIALDNGAYGSTGSQETCALRYIDLEIFANACGIQNTAKVNSKEGVIEAFRKFKAMRELSFIHVILKPGNTNAPNIPMSPEEATKRFKETLDVKKF

SEQ ID NO: 7 - protein, 524 aa, Methanocaldococcus jannaschii
MVKILVTDPLHEDAIKILEEVGEVEVATGLTKEELLEKIKDADVLVVRSGTKVTRDVIEKAEKLKVIGRAGVGVDNIDVEAATEKGIIVVNAPDASSISVAELTMGLMLAAARNIPQATASLKRGEWDRKRFKGIELYGKTLGVIGLGRIGQQVVKRAKAFGMNIIGYDPYIPKEVAESMGVELVDDINELCKRADFITLHVPLTPKTRHIIGREQIALMKKNAIIVNCARGGLIDEKALYEALKEGKIRAAALDVFEEEPPKDNPLLTLDNVIGTPHQGASTEEAQKAAGTIVAEQIKKVLRGELAENVVNMPNIPQEKLGKLKPYMLLAEMLGNIVMQVLDGSVNRVELIYSGELAKEKTDLIKRAFLKGLLSPILLAGINLVNAPIIAKNRNINVVESSTSEEKYGNAIKITAESDKKKFSIVGAIINNKPVILEVDGYEVSFIPEGVLAIIKHIDRPGTIGRVCITLGDYGINIASMQVGRKEPGGESVMLLNLDHTVPEEVIEKIKEIPNIKDVAVINL

SEQ ID NO: 8 - protein, 370 aa, Methanosarcina barkeri
MKPTRVPNNPCFSSGPCAKHPGYSIEELKDTPFGRSHRSNLGKEKLAEAIKKTRDMLGLPDDYLVGIVPASDTGAFEMCLWSMLGCRGVDVLVWESFSKGWATDITKQLKLKDVRVFEAEYGKLPDLKKVDFKNDVVFVWNGTTSGVKVPNGDWIPENREGLTLCDATSAIFAMDIPYHKLDVITFSWQKVLGGEGAHGMLILSPRAVQRLESYTPAWPLPKIFRLTKGGKLNKKIFEGSTINTPSMLANEDWLATLKWAESVGGLKPLIQRTNDNLAVFEAFVAKNNWIHFLAETKEIRSSTSVCFKVDLSDEKLKELIKTLEKEKVAYDIGSYRDAPSGLRIWCGATVEKEDLQCLCEWIEWAYNLVK

SEQ ID NO: 9 - protein, 430 aa, Methanoculleus marisnigri
MREKYLLHCPGCGRLFPDNYTLDCPLGCNALLRTVYAEHRLTLRDLPGIFRYSSWLPIEGHLRIDAGPVSYASEGLARELGLSNLTVTFSGYWPERGGRMETCSFKELEAQPTVLRLGEKGAGVLQISSAGNTGRAFCQVSALTGAPVVVVVPASAADRLWTTVPAPNVCLITVEGDYSDSIAFGREVCSLPGIVPEGGAKNVARRDGMGTVMLDAALFAGRLPDAYFQAIGSGTGGIAAWEAAERLVADGRFGSRLPTLHLSQNLPFVPMVRAWEAGRREIVPEVDMPDAEASIVRVSADVLTNRHPPWEVRGGVYDALAASGGRMYAVANDDTRSAGRLFEATEEIDLDPAAAVAVASLIRAAEEGFIGPDDHILLNVTGGGYARAAEDLDRYPVEPYLRVRAGEAFAGDVRDAVRGWLAEQEVVVRA

SEQ ID NO: 10 - protein, 375 aa, Methanocaldococcus jannaschii
MISSRCKNIKPSAIREIFNLATSDCINLGIGEPDFDTPKHIIEAAKRALDEGKTHYSPNNGIPELREEISNKLKDDYNLDVDKDNIIVTCGASEALMLSIMTLIDRGDEVLIPNPSFVSYFSLTEFAEGKIKNIDLDENFNIDLEKVKESITKKTKLIIFNSPSNPTGKVYDKETIKGLAEIAEDYNLIIVSDEVYDKIIYDKKHYSPMQFTDRCILINGFSKTYAMTGWRIGYLAVSDELNKELDLINNMIKIHQYSFACATTFAQYGALAALRGSQKCVEDMVREFKMRRDLIYNGLKDIFKVNKPDGAFYIFPDVSEYGDGVEVAKKLIENKVLCVPGVAFGENGANYIRFSYATKYEDIEKALGIIKEIFE

SEQ ID NO: 11 - protein, 251 aa, Methanocaldococcus jannaschii [sequence omitted]

SEQ ID NO: 12 - protein, 224 aa, Methanocaldococcus jannaschii
MITLCNRFTEYKCGNVAIVVDVLRASTTITTLLSFIDEVYITTSTSKKENAIYIGERKGRKIEGFDFGNSPTEILANKDIIKERYENGEKVILTTTNGTRVLKSLDAEHIFIGAIVNAKYVAKAVEDFEDVSLVPCHRENNFAIDDFIGCGVIAKYLNGEFDEFIKAALELTKHDWMSLILNSSSAENLKNLGYEKDVTFAILENSIDAVGIYKKDKSKVVRFK

SEQ ID NO: 13 - protein, 333 aa, Chromohalobacter israelensis
MSRMISLREAETLAVAALEAVGVPRWEAEVTARALIDAERDGLASHGLSRLPFYLAQARSGKVVADAQARVEVAGSVIRVDARHGLAFPAIARGVERAIPLARELGLVAVAIGGSHHFGVAGAPVERLAREGLVAMAFSNAPSAMAPWGGKRPLYGTNPIAFATPRRGTDPLVIDLSLSKVARGKVMLAKKAGEPIPEGWALDIEGRPTTDPDAAIAGSMVPAGDAKGASLALMVELLTAGLTGSHFGFQASSFFEPEGEAPSVGHLMLAFDPAHFSDGYLEHIEALFQAMLEQEGVRLPGTRRHALRRERGESLELPEAVVDELRAYAVSRV

SEQ ID NO: 14 - protein, 608 aa, Castellaniella denitrificans
MANDTRQVVQGVQEMTPSEAFVETMVANGVTEIFGIMGSAFMDAMDIFAPAGIKLIPVVHEQGAAHMADGFARVSGRTGVVIGQNGPGISNCVTAIAAAYWAHTPVVIVTPEAGTTGIGLGGFQEARQLPMFQEFTKYQGHVTHPARMAEYTARCFARARDEMGPAQLNIPRDYFYGKIKCEIPLPQPLDRGPGGAQSLDAAARLLAEAKFPVIISGGGVVMGDAVEECKALAERLGAPVVNSYLHNDSFPASHPLWCGPLGYQGSKAAMKLLADADVVLALGTRLGPFGTLPQHGLDYWPKNARIIQVDADSKMLGLVKKITVGVCGDAKASAAEISRRIDGMKLACDANKAERAARIQAEKDAWEQELTDWTHERDPFSLDMIEEQSKEEGNWLHPRQVLRELEKAMPEDVMVSTDIGNINSVANSYLRFEKPRSFFAAMSWGNCGYAFPTIIGAKVAAPHRPAVSYAGDGAWGMSMSEIMTCVRHDIPVTAVVFHNRQWGAEKKNQVDFYNRRFVAGELESESFAGIARAMGAEGVVVDRIEDVGPALKKAIDAQMNDRKTTVIEIMCTRELGDPFRRDALSKPVRLLEKYRDYTGGGGHHHHHH

SEQ ID NO: 15 - protein, 397 aa, Methanosarcina acetivorans
MYVVNPEEKVIEIMKQTGIDLAATLPCDRIKNLLPLVSENFPEIKLTREENGVGICAGIYLAGGKPMMLIQSTGLGNMINALESLNVTCKIPLPILASWRGVYKEGIEAQVPLGAHLPSILEGAGLTYTIIGETEKLPLLENVILDAFENSRPHIALVSPKVWEASECCAWQAAGMPIKPEIMERTCRFSLTSGTLKPFMLRNDAICTLASELDDEITVTNLGVPCKELYACRDRELNFYMFGSMGLVSSIGLGLALRSEKTVITFDGDGSLLMNPNALLEIAKEAPKNLIIIALDNGAYGSTGSQETCALRYIDLEIFANACGIQNTAKVNSKEGVIEAFRKFKAMRELSFIHVILKPGNTNAPNIPMSPEEATKRFKETLDVKKFGGGGHHHHHH

SEQ ID NO: 16 - protein, 534 aa, Methanocaldococcus jannaschii
MVKILVTDPLHEDAIKILEEVGEVEVATGLTKEELLEKIKDADVLVVRSGTKVTRDVIEKAEKLKVIGRAGVGVDNIDVEAATEKGIIVVNAPDASSISVAELTMGLMLAAARNIPQATASLKRGEWDRKRFKGIELYGKTLGVIGLGRIGQQVVKRAKAFGMNIIGYDPYIPKEVAESMGVELVDDINELCKRADFITLHVPLTPKTRHIIGREQIALMKKNAIIVNCARGGLIDEKALYEALKEGKIRAAALDVFEEEPPKDNPLLTLDNVIGTPHQGASTEEAQKAAGTIVAEQIKKVLRGELAENVVNMPNIPQEKLGKLKPYMLLAEMLGNIVMQVLDGSVNRVELIYSGELAKEKTDLIKRAFLKGLLSPILLAGINLVNAPIIAKNRNINVVESSTSEEKYGNAIKITAESDKKKFSIVGAIINNKPVILEVDGYEVSFIPEGVLAIIKHIDRPGTIGRVCITLGDYGINIASMQVGRKEPGGESVMLLNLDHTVPEEVIEKIKEIPNIKDVAVINLGGGGHHHHHH

SEQ ID NO: 17 - protein, 380 aa, synthetic construct
MKPTRVPNNPCFSSGPCAKHPGYSIEELKDTPFGRSHRSNLGKEKLAEAIKKTRDMLGLPDDYLVGIVPASDTGAFEMCLWSMLGCRGVDVLVWESFSKGWATDITKQLKLKDVRVFEAEYGKLPDLKKVDFKNDVVFVWNGTTSGVKVPNGDWIPENREGLTLCDATSAIFAMDIPYHKLDVITFSWQKVLGGEGAHGMLILSPRAVQRLESYTPAWPLPKIFRLTKGGKLNKKIFEGSTINTPSMLANEDWLATLKWAESVGGLKPLIQRTNDNLAVFEAFVAKNNWIHFLAETKEIRSSTSVCFKVDLSDEKLKELIKTLEKEKVAYDIGSYRDAPSGLRIWCGATVEKEDLQCLCEWIEWAYNLVKGGGGHHHHHH

SEQ ID NO: 18 - protein, 440 aa, synthetic construct
MREKYLLHCPGCGRLFPDNYTLDCPLGCNALLRTVYAEHRLTLRDLPGIFRYSSWLPIEGHLRIDAGPVSYASEGLARELGLSNLTVTFSGYWPERGGRMETCSFKELEAQPTVLRLGEKGAGVLQISSAGNTGRAFCQVSALTGAPVVVVVPASAADRLWTTVPAPNVCLITVEGDYSDSIAFGREVCSLPGIVPEGGAKNVARRDGMGTVMLDAALFAGRLPDAYFQAIGSGTGGIAAWEAAERLVADGRFGSRLPTLHLSQNLPFVPMVRAWEAGRREIVPEVDMPDAEASIVRVSADVLTNRHPPWEVRGGVYDALAASGGRMYAVANDDTRSAGRLFEATEEIDLDPAAAVAVASLIRAAEEGFIGPDDHILLNVTGGGYARAAEDLDRYPVEPYLRVRAGEAFAGDVRDAVRGWLAEQEVVVRAGGGGHHHHHH

SEQ ID NO: 19 - protein, 385 aa, synthetic construct
MISSRCKNIKPSAIREIFNLATSDCINLGIGEPDFDTPKHIIEAAKRALDEGKTHYSPNNGIPELREEISNKLKDDYNLDVDKDNIIVTCGASEALMLSIMTLIDRGDEVLIPNPSFVSYFSLTEFAEGKIKNIDLDENFNIDLEKVKESITKKTKLIIFNSPSNPTGKVYDKETIKGLAEIAEDYNLIIVSDEVYDKIIYDKKHYSPMQFTDRCILINGFSKTYAMTGWRIGYLAVSDELNKELDLINNMIKIHQYSFACATTFAQYGALAALRGSQKCVEDMVREFKMRRDLIYNGLKDIFKVNKPDGAFYIFPDVSEYGDGVEVAKKLIENKVLCVPGVAFGENGANYIRFSYATKYEDIEKALGIIKEIFEGGGGHHHHHH

SEQ ID NO: 20 - protein, 261 aa, synthetic construct
MKAFEFLYEDFQRGLTVVLDKGLPPKFVEDYLKVCGDYIDFVKFGWGTSAVIDRDVVKEKINYYKDWGIKVYPGGTLFEYAYSKGKFDEFLNECEKLGFEAVEISDGSSDISLEERKNAIKRAKDNGFMVLTEVGKKMPDKDKQLTIDDRIKLINFDLDAGADYVIIEGRESGKGIGLFDKEGKVKENELDVLAKNVDINKVIFEAPQKSQQVAFILKFGSSVNLANIAFDEVISLETLRRGLRGDTFGKVGGGGHHHHHH

SEQ ID NO: 21 - protein, 234 aa, synthetic construct
MITLCNRFTEYKCGNVAIVVDVLRASTTITTLLSFIDEVYITTSTSKKENAIYIGERKGRKIEGFDFGNSPTEILANKDIIKERYENGEKVILTTTNGTRVLKSLDAEHIFIGAIVNAKYVAKAVEDFEDVSLVPCHRENNFAIDDFIGCGVIAKYLNGEFDEFIKAALELTKHDWMSLILNSSSAENLKNLGYEKDVTFAILENSIDAVGIYKKDKSKVVRFKGGGGHHHHHH

SEQ ID NO: 22 - protein, 343 aa, synthetic construct
MSRMISLREAETLAVAALEAVGVPRWEAEVTARALIDAERDGLASHGLSRLPFYLAQARSGKVVADAQARVEVAGSVIRVDARHGLAFPAIARGVERAIPLARELGLVAVAIGGSHHFGVAGAPVERLAREGLVAMAFSNAPSAMAPWGGKRPLYGTNPIAFATPRRGTDPLVIDLSLSKVARGKVMLAKKAGEPIPEGWALDIEGRPTTDPDAAIAGSMVPAGDAKGASLALMVELLTAGLTGSHFGFQASSFFEPEGEAPSVGHLMLAFDPAHFSDGYLEHIEALFQAMLEQEGVRLPGTRRHALRRERGESLELPEAVVDELRAYAVSRVGGGGHHHHHH

SEQ ID NO: 23 - DNA, 1350 bp, synthetic construct
  source 1..1350: mol_type = other DNA
atgaatggtgcactggataataccgagaaaccgcgtaccattcaagaaatccaggttaaaattgatgccggtgaagcagttgttctgacagccgaagaaattggtgcaaaagttcgtgccggtggtgaaattcgtctggaagatattgatgttgttaccaccgcaacacgtggcattatgagcggcacctatgcagtttttagctttaaagttagcgaaccggatagctttgttaaagcaagcaaagttctgctgaatggtgttccggcaattgttggtccgtgtccgaatgaacgtctgggtgttctggatctgattgttctgggcaccgcacatagcgaaaccgatagccgttatggtggtggtcacctgtttcgtgatatggttgaaggtaaaagcattaccgttgatgtgaccacctcagttggtgatttcttcagcgttgaaacccgtctgagcgaaatgccgtttgcacgtctgtatgcaacccgtcatgcctttaagaactatcgtgcatttgtgaatccgggtaaagaaagcatcaaaaccatatttcatgccctgccgtttgaaggtgaatttcgtgaactgaccttttgtggttgtggtgaactgaacccgattcagaatgatccgaaactggaaaccattggtattggcacccgtgttctgattaatggtgcagaaggttttgttaccggtcagggcacccgttcagcaccggataacccgaatctgaccggttttgccgatctgcatgatatgacaccggaatatatgggtggttttgtgaccagcgcaggtccggaaattgttaatacctgggcagttccgattccggttctgagccgtgaaatgctggaaaatattctgcagctggatagccagattccgctgaaactggttgatctggcaggtcgtctgccgctgtgtgaaattacctatggtgatgtttgggataatacagacctgagcgttaaatatgagccggagaaatgcatcgattgcaaaatttgttgtgttgccgaagcatgtccgatgggtgcagttagcaaaggtgaaaatggtgcggttcataatccggaactgtgttttaattgtggtctgtgtattagccgctgtaaaggtgaagcatttagcgcaaaactgggtacagtgagctgtgcagttggtggttgctttcgcgatattaaagttaccctgcgtcagagcgatcgtgcacgtgccgttaaagcagcagaagaactgaaagcacaggtgctgagcggtaaatttcgtctgaaagaaccggttgagaaaattagctggcgtgaaggtggcggtggtcatcaccatcatcatcactgatga

SEQ ID NO: 24 - DNA, 1404 bp, synthetic construct
  source 1..1404: mol_type = other DNA
atgacctatgataaagcagaactggttgccctggataagaagtatgtttggcatcatctgacccagcacaagaactttgaaccggcaatttatgtgaaaggtgaaggtatgcgtatcaccgatattgatggtaaaacctatctggatgcagttagcggtggtgtttggaccgttaatgttggttatggtcgtaaagaaattgttgatgccgttgcaaagcagatgatggaaatgtgttattttgccaatggtattggtaatgtgccgaccatcaaatttagcgagaaactgattagcaagatgcctggtatgagccgtgtttatctgagcaatagcggtagcgaagcaaatgagaaagcctttaaaatcgtgcgtcagattggtcagctgaaacatggtggtaagaagaccggtattctgtatcgtgcacgtgattatcatggcaccaccattggtacactgagcgcatgtggtcagtttgaacgtaaagttcagtatggtccgtttgcaccgggtttctatgaatttccggattgtgatgtgtaccgcagcaaatttggtgattgtgcagatctgggtgttaaaatggcaaaacagctggaagaagttattctgaccgttggtccggatgaactgggtgcagttattgttgaaccgatgaccgcaggcggtggtattctggttccgcctgcaggttattatgaaaccattcgtgaaatctgcgacaaatatgaactgctgctgattattgatgaagttgtttgtggtctgggtcgtaccggtaaatggtttggttatcagcatttcaatgtgcagccggatattgttaccatggccaaaggtgttgcaagcggttatgcaccgattagctgtaccgttaccaccgagaaagtatttcaggattttgtgaatgatccggcagataccgatgcatattttcgtgatattagcacctttggtggttgtaccagcggtccggcagcagcactggcaaatattgaaattattgaacgcgaaaacctgctggaaaactgtaccaaaatgggtgatcgtctgctggaaggtctgaaaggcctgatggcgaaacatccgattattggtgatgttcgtggtaaaggtctgtttgcaggcattgaaattgttaaagatcgtgcaaccaaagaaccgattgccgaagcagttgcaaatgcaatggttggtgcagcaaaacaggcaggcgttctgattggtaaaacaagccgtagctttcgcgaatttaacaataccctgacactgtgtccggcactgattgcaaccgaagcagatatcgatgaaattgtggcaggtatcgataaagcatttaccaccgttgaacagaaatttggcctgggtggtggcggtcatcatcaccatcatcattgatga

SEQ ID NO: 25 - DNA, 1870 bp, synthetic construct
  source 1..1870: mol_type = other DNA
agataggcggaagctatttatacggttaaggaggtttcatatggcaaatgatacccgtcaggttgttcagggtgttcaagaaatgaccccgagcgaagcatttgttgaaaccatggtggcaaatggtgtgaccgaaatatttggtattatgggtagcgcatttatggatgccatggatatatttgcaccggcaggtattaaactgattccggttgtgcatgaacagggtgcagcacacatggcagatggttttgcacgtgttagcggtcgtaccggtgttgttattggtcagaatggtccgggtattagcaattgtgttaccgcaattgcagcagcatattgggcacatacaccggttgtgattgttacaccggaagcaggcaccaccggtattggtttaggtggttttcaagaagcacgtcagctgccgatgtttcaagagttcaccaaatatcagggtcatgttacccatccggcacgtatggccgaatataccgcacgttgttttgcccgtgcacgtgatgaaatgggtcctgcacagctgaatattccgcgtgattatttctacggcaaaatcaaatgtgaaattccgctgccgcagccgctggatcgtggtcctggtggtgcacagagcctggatgcagcagcccgtctgctggcagaagcaaaatttccggttattatcagcggtggtggtgttgtgatgggtgatgcagttgaagaatgtaaagcactggcagaacgtctgggtgcaccggtggttaatagctatctgcataatgatagctttccggcaagccatccgctgtggtgtggtccgctgggttatcagggtagcaaagcagcaatgaaactgctggccgatgcagatgttgttctggcactgggcacccgtctgggtccgtttggtacactgcctcagcatggtctggattattggccgaagaatgcacgtattattcaggttgatgccgacagcaaaatgctgggtttagtgaagaaaatcaccgttggtgtttgtggtgatgccaaagcaagcgcagcagaaattagccgtcgtattgatggtatgaaactggcatgtgatgcaaataaagcagaacgtgcagcacgtattcaggcagagaaagatgcatgggaacaagaactgaccgattggacccatgaacgtgatccgtttagcctggatatgattgaagaacagagcaaagaagaaggcaattggctgcatccgcgtcaggttctgcgtgaactggagaaagcaatgccggaagatgttatggttagcaccgatattggtaacattaatagcgtggccaattcctatctgcgttttgagaaaccgcgtagcttctttgcagccatgagctggggtaattgtggttatgcatttccgaccattattggtgcaaaagttgcagcaccgcatcgtccggcagttagctatgccggtgatggtgcatggggtatgagcatgagcgaaattatgacctgtgttcgtcatgatattccggttaccgcagttgtgtttcataatcgtcagtggggtgcagaaaagaagaatcaggtggatttctacaaccgtcgttttgttgcaggcgaactggaatcagagagctttgcaggtattgcacgtgccatgggtgccgaaggtgttgttgttgatcgtattgaagatgttggtcctgcgctgaagaaagccattgatgcacagatgaatgatcgtaaaaccaccgtgattgaaatcatgtgcacacgcgaactgggcgatccgtttcgtcgtgatgcactgagcaaaccggtgcgcctgctggagaaatatcgtgattatacaggtggtggcggtcatcatcaccatcatcattgataa

SEQ ID NO: 26 - DNA, 1197 bp, synthetic construct
  source 1..1197: mol_type = other DNA
atgtatgtggtgaacccggaagagaaagtgatcgaaattatgaaacagaccggcattgatctggcagcaaccctgccgtgtgatcgtatcaagaatctgctgccgctggttagcgaaaacttcccggaaattaaactgacgcgtgaagaaaatggtgttggtatttgtgcaggtatttatctggcaggcggtaaaccgatgatgctgattcagagcaccggtctgggtaatatgattaatgcactggaaagcctgaatgtgacctgtaaaattccgctgccgattctggcaagctggcgtggtgtttataaagaaggtattgaagcacaggttccgctgggtgcacatctgccgagcattctggaaggtgcgggtctgacctataccattattggtgaaaccgagaaactgccgctgctggaaaatgttattctggatgcatttgaaaattcccgtccgcatattgcactggttagcccgaaagtttgggaagcaagcgaatgttgtgcatggcaggcagcaggtatgccgattaaaccggaaatcatggaacgtacctgtcgttttagcctgaccagcggtacactgaaaccgtttatgctgcgtaatgatgcaatttgtaccctggcaagcgaactggatgatgaaattaccgttaccaatctgggtgttccgtgtaaagaactgtatgcatgtcgtgatcgtgagctgaacttttatatgtttggtagcatgggtcttgtgagcagcattggtctgggtttagcactgcgtagcgaaaagaccgttattacctttgatggtgatggtagcctgctgatgaatccgaatgcactgttagaaattgcaaaagaagcaccgaagaacctgattattatcgcactggataatggtgcgtacggtagcaccggtagccaagaaacctgtgcactgcgttatatcgatctggaaatctttgcaaatgcctgcggcattcagaataccgcaaaagttaatagcaaagaaggtgtgattgaagccttccgtaaattcaaagcaatgcgtgaactgagctttatccatgtgattctgaaaccgggtaataccaatgcaccgaatattccgatgagtccggaagaagcaaccaaacgttttaaagaaaccctggacgtgaagaagtttggtggtggcggtcatcatcaccatcatcattgatga

SEQ ID NO: 27 - DNA, 1619 bp, synthetic construct
  source 1..1619: mol_type = other DNA
ggaggtgtacaatggtgaaaattctggttaccgatccgctgcatgaagatgcaatcaaaatcctggaagaagttggcgaagttgaagttgcaaccggtctgaccaaagaagaactgctggagaagatcaaagatgccgatgttctggttgttcgtagcggcaccaaagttacccgtgatgttattgagaaggccgagaaactgaaagttattggtcgtgccggtgttggtgttgataatattgatgttgaagcagccaccgagaagggcattattgttgttaatgcaccggatgcaagcagcattagcgttgcagaactgaccatgggtctgatgctggcagcagcacgtaatattccgcaggcaaccgcaagcctgaaacgtggtgaatgggatcgtaaacgttttaaaggcattgagctgtatggtaaaaccctgggtgtgattggtctgggtcgtattggtcagcaggttgttaaacgtgcaaaagcatttggcatgaacatcattggttatgatccgtatattccgaaagaagttgcagaaagcatgggtgttgaactggtggatgatattaatgaactgtgtaaacgtgccgattttatcaccctgcatgttccgctgacaccgaaaacacgtcatatcattggtcgcgagcagattgcactgatgaagaagaacgccattattgtgaattgtgcacgcggtggtctgatcgatgagaaagcactgtatgaagcactgaaagaaggtaaaattcgcgcagcagccctggatgtctttgaagaagaaccgcctaaagataatccgctgctgaccctggataatgttattggtacaccgcatcagggtgcaagcaccgaagaagcacagaaagcagcaggcaccattgttgcagagcagatcaagaaagttctgcgtggtgaactggcagaaaatgttgtgaatatgccgaacattccgcaagagaaactgggtaaactgaaaccgtatatgctgctggccgaaatgctgggtaatattgttatgcaggttctggatggtagcgttaatcgtgtggaactgatttattcaggcgaactggcaaaagaaaagaccgatctgattaaacgcgcatttctgaaaggtctgctgagcccgattctgctggcaggtattaatctggtgaatgcaccgattattgccaagaaccgtaatatcaatgttgtcgaaagctcgaccagcgaagagaaatatggtaacgccatcaaaattaccgcagagagcgataagaagaaattcagcattgtgggtgccatcatcaataacaaaccggttattctggaagtggatggttatgaagttagctttattccggaaggtgttctggccattatcaaacatattgatcgtccgggtacaattggccgtgtttgtattaccctgggcgattatggtattaacattgcaagcatgcaggttggtcgtaaagaacctggtggtgaaagcgtgatgctgctgaatctggatcacaccgtgccggaagaagtgattgaaaagattaaagaaatcccgaacatcaaagacgtggccgtgattaatctgggcggtggtggtcatcaccatcaccatcattgataa

SEQ ID NO: 28 - DNA, 1160 bp, synthetic construct
  source 1..1160: mol_type = other DNA
ggaggagctcatgaaaccgacacgtgttccgaataatccgtgttttagcagcggtccgtgtgcaaaacatccgggttatagcattgaagaactgaaagataccccgtttggtcgtagccatcgtagcaatctgggtaaagagaaactggcagaagccatcaagaagacccgtgatatgctgggtctgccggatgattatctggttggtattgttccggcaagcgataccggtgcatttgaaatgtgtctgtggtcaatgctgggttgtcgtggtgttgatgttctggtttgggaaagttttagcaaaggttgggcaaccgatattaccaaacagctgaaactgaaggatgtgcgtgtcttcgaagccgaatatggtaaactgccggatctgaagaaggtggatttcaagaacgatgtggtgtttgtgtggaatggtacaaccagcggtgttaaagttccgaatggtgattggattccggaaaatcgtgaaggtctgaccctgtgtgatgcaaccagcgcaatctttgcaatggatatcccgtatcataaactggatgtgattacctttagctggcagaaagttttaggtggtgaaggtgcacatggtatgctgattctgagtccgcgtgcagttcagcgtctggaaagctatacaccggcatggcctctgccgaaaatctttcgtctgaccaaaggtggcaaactgaacaagaagatctttgaaggcagcaccattaataccccgagcatgctggcaaatgaagattggctggcaaccctgaaatgggcagaaagcgttggtggtctgaaaccgctgattcagcgtaccaatgataatctggcagtgtttgaagcatttgtggccaagaataactggattcattttctggccgaaaccaaagaaattcgtagcagcaccagcgtttgctttaaagttgatctgagtgacgaaaagctgaaagagctgattaaaaccctggagaaagaaaaggtggcctatgatattggtagctatcgtgatgcaccgagcggtctgcgtatttggtgtggtgcaaccgttgagaaagaagatctgcagtgtctgtgtgaatggattgaatgggcatataacctggtgaaaggtggtggcggccaccatcatcaccaccactgataagctt

SEQ ID NO: 29 - DNA, 1375 bp, synthetic construct
  source 1..1375: mol_type = other DNA
aatatactctatagagaaaattaagggcgtctttttaaggaggttacatatgcgcgagaagtaccttcttcattgtccaggttgtggacgtttgttcccggataattacacattggattgtccgttaggatgcaatgctttgctgcgcacagtttatgcggaacaccgcttaaccttgcgcgacttgcctggcatcttccgctattcatcctggcttcctatcgagggacatttacgcatcgatgccggacccgtgagttacgcaagtgaaggtttggctcgtgaattggggttgtctaaccttaccgtcaccttcagtggatattggccggagcgcggtggacgcatggaaacttgttctttcaaagagctggaagcccaaccaacggtacttcgtttgggtgaaaagggagccggcgtccttcagatcagcagtgccggaaacacgggccgtgccttctgtcaagtttcagcccttacgggtgctccggtagttgtagttgtacctgccagtgcagcagatcgcttgtggacgacggtgccagcacctaacgtttgtctgatcacggtagaaggcgactacagcgacagcatcgctttcgggcgtgaggtatgttcccttccaggtattgtacccgagggaggggcgaagaacgtcgctcgtcgcgacggaatgggtaccgtaatgttagatgcagcccttttcgctggacgcttgcctgacgcatactttcaagccattgggtctggcacgggcggcattgctgcatgggaagcggccgagcgtttagtggcagatggacgcttcgggtcgcgcttgcctacactgcacttatctcagaacttaccgtttgtgcctatggttcgtgcctgggaagccggccgtcgtgaaattgtccccgaggtggatatgccagatgcagaggcgtctatcgtacgtgtatcggcagacgttctgaccaaccgccatccgccttgggaggttcgtggcggcgtttacgacgcccttgctgcatcaggtggtcgtatgtatgctgtcgctaatgacgatacccgttcggcaggtcgtcttttcgaagctacggaagaaattgacctggacccagccgccgccgtcgctgttgcctctctgattcgcgcagctgaagaaggattcattggcccggatgaccatattttgttgaacgtcactggcggtggatatgcgcgcgcagcagaggacctggatcgttaccccgtcgagccctatcttcgcgttcgcgcgggtgaagcatttgccggggatgtacgtgacgccgtccgtggttggctggctgaacaggaggtggtagtccgcgccggcggcggcggtcaccaccatcatcaccattgataa

SEQ ID NO: 30 - DNA, 1176 bp, synthetic construct
  source 1..1176: mol_type = other DNA
ggaggctcgagatgattagcagccgctgcaagaacattaaaccgagcgcaattcgcgaaatctttaatctggcaaccagcgattgtattaatcttggtattggtgaaccggatttcgataccccgaaacatattattgaagcagcaaaacgtgcactggatgaaggtaaaacccattatagcccgaataatggtattccggaactgcgtgaagaaatcagcaataaactgaaagatgactacaacctggatgtggacaaagataacattattgttacctgtggtgcaagcgaagcactgatgctgagcattatgaccctgattgatcgtggtgatgaagttctgattccgaatccgagctttgttagctattttagcctgaccgaatttgccgaaggcaaaatcaagaatatcgatctggatgagaacttcaacatcgacctggagaaagtgaaagagagcatcaccaagaagaccaaactgatcatctttaacagcccgagcaatccgaccggtaaagtttatgataaagaaaccattaaaggcctggccgaaattgcggaagattataacctgattattgtgagcgacgaggtgtacgataaaatcatctatgacaagaaacactacagcccgatgcagtttaccgatcgttgtattctgattaacggcttcagcaaaacctatgcaatgaccggttggcgtattggttatctggcagttagtgatgaactgaacaaagaactggatctgatcaacaacatgatcaagatccaccagtatagctttgcatgtgcaaccacctttgcacagtatggtgcactggcagcactgcgtggtagccagaaatgtgttgaagatatggttcgcgagtttaaaatgcgtcgtgatctgatttataacggcctgaaagacatcttcaaagtgaataaaccggatggtgccttttatatctttccggatgttagcgaatatggtgatggtgttgaagtggccaagaagctgattgaaaataaggttctgtgtgttccgggtgttgcatttggtgaaaatggtgcaaactatatccgttttagctacgccaccaaatatgaggatattgagaaagccctgggcatcatcaaagaaatctttgaaggcggcggtggtcaccatcatcatcatcactgataagctt

SEQ ID NO: 31 - DNA, 824 bp, synthetic construct
  source 1..824: mol_type = other DNA
taacggcaacgtagttagtagtaaggaggttacatatgaaagcctttgagttcctgtatgaagattttcagcgtggtctgaccgttgttctggataaaggtctgcctccgaaatttgttgaggattatctgaaagtgtgcggagattatatcgactttgtgaaatttggttggggcaccagcgcagttattgatcgtgatgttgtgaaagagaaaatcaactactataaggactggggcatcaaagtttatcctggtggcaccctgtttgaatatgcctatagcaaaggcaaattcgacgagtttctgaacgaatgtgagaaactgggttttgaagccgtggaaattagtgatggtagcagcgatattagcctggaagaacgtaagaatgcaatcaaacgtgccaaagataacggctttatggttctgaccgaagtgggcaagaagatgccggataaagataaacagctgaccattgatgatcgcatcaaactgattaactttgatctggatgccggtgccgattatgttattattgaaggtcgtgaaagcggtaaaggtattggcctgtttgataaagagggcaaagtcaaagaaaacgaactggatgttctggccaagaacgtggatattaacaaagtgatctttgaagcaccgcagaaaagccagcaggttgcatttattctgaaattcggtagcagtgtgaatctggccaatattgcatttgatgaagttatcagcctggaaaccctgcgtcgtggtctgcgtggtgatacctttggtaaagttggcggtggtggtcatcaccaccaccatcattgataa

SEQ ID NO: 32 - DNA, 719 bp, synthetic construct
  source 1..719: mol_type = other DNA
ggaggatccttatgattaccctgtgcaatcgcttcaccgagtataaatgtggtaatgttgccattgttgttgatgttctgcgtgcaagcaccaccattacaaccctgctgagctttattgatgaagtgtatattaccacgagcaccagcaagaaagaaaacgccatctatattggtgaacgcaaaggtcgtaaaatcgagggttttgattttggtaatagcccgaccgaaattctggccaacaaagatattatcaaagaacgctatgagaacggcgaaaaggttattctgaccaccaccaatggcacccgtgttctgaaaagcctggatgcagaacacatctttatcggtgcaattgtgaacgccaaatatgttgcaaaagccgtggaagattttgaagatgttagcctggttccgtgtcatcgtgaaaataactttgccatcgatgattttattggctgcggtgtgattgccaaatatctgaatggtgagttcgacgagtttatcaaagcagcactggaactgaccaaacatgattggatgagcctgattctgaatagcagcagcgcagaaaatctgaagaatctgggctatgaaaaggatgtgaccttcgccattctggaaaatagcattgatgcagtgggcatctacaagaaagacaaaagcaaagtggtgcgctttaaaggtggtggcggccaccatcatcaccaccactgataa

SEQ ID NO: 33 - DNA, 1050 bp, synthetic construct
  source 1..1050: mol_type = other DNA
ggaggactagtatgagccgtatgattagcctgcgtgaagcagaaaccctggcagttgcagcactggaagcagttggtgttccgcgttgggaagccgaagttaccgcacgtgcactgattgatgcagaacgtgatggtctggcaagccatggtctgagccgtctgccgttttatctggcacaggcacgtagtggtaaagttgttgcagatgcccaggcacgtgttgaagttgcaggtagcgttattcgtgttgatgcacgtcatggcctggcatttccggcaattgcacgtggtgttgaacgtgccattccgctggcacgtgaactgggtttagttgcagttgcaattggtggtagccatcattttggtgttgcgggtgcaccggttgaacgtctggcacgcgaaggtctggttgcaatggcatttagcaatgcaccgagcgcaatggcaccgtggggtggtaaacgtccgctgtatggcaccaatccgattgcatttgcaacaccgcgtcgtggcaccgatccgctggttattgatctgagcctgagcaaagttgcacgcggtaaagttatgctggcaaagaaggccggtgaaccgattccggaaggttgggcattagatattgaaggtcgtccgaccacagatccggatgcagcaattgcgggtagcatggttcctgccggtgatgcaaaaggtgcaagcctggcactgatggttgaactgctgaccgcaggtctgaccggtagccattttggttttcaggcaagcagcttctttgaaccggaaggtgaagcaccgagtgttggtcatctgatgctggcatttgatccggcacattttagtgatggttatctggaacatatcgaagcactgtttcaggccatgctggaacaagaaggtgttcgtctgcctggtacacgtcgtcatgcactgcgtcgtgaacgtggtgaaagtctggaactgccggaagccgttgttgatgaactgcgtgcatacgccgttagccgtgttggcggcggcggtcaccaccatcatcaccattgataagctt

SEQ ID NO: 34 - DNA, 7978 bp, synthetic construct
  source 1..7978: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtgagctcatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcatcggatcctcctcatcaatgatgatggtgatgatgaccgccaccacccaggccaaatttctgttcaacggtggtaaatgctttatcgatacctgccacaatttcatcgatatctgcttcggttgcaatcagtgccggacacagtgtcagggtattgttaaattcgcgaaagctacggcttgttttaccaatcagaacgcctgcctgttttgctgcaccaaccattgcatttgcaactgcttcggcaatcggttctttggttgcacgatctttaacaatttcaatgcctgcaaacagacctttaccacgaacatcaccaataatcggatgtttcgccatcaggcctttcagaccttccagcagacgatcacccattttggtacagttttccagcaggttttcgcgttcaataatttcaatatttgccagtgctgctgccggaccgctggtacaaccaccaaaggtgctaatatcacgaaaatatgcatcggtatctgccggatcattcacaaaatcctgaaatactttctcggtggtaacggtacagctaatcggtgcataaccgcttgcaacacctttggccatggtaacaatatccggctgcacattgaaatgctgataaccaaaccatttaccggtacgacccagaccacaaacaacttcatcaataatcagcagcagttcatatttgtcgcagatttcacgaatggtttcataataacctgcaggcggaaccagaataccaccgcctgcggtcatcggttcaacaataactgcacccagttcatccggaccaacggtcagaataacttcttccagctgttttgccattttaacacccagatctgcacaatcaccaaatttgctgcggtacacatcacaatccggaaattcatagaaacccggtgcaaacggaccatactgaactttacgttcaaactgaccacatgcgctcagtgtaccaatggtggtgccatgataatcacgtgcacgatacagaataccggtcttcttaccaccatgtttcagctgaccaatctgacgcacgattttaaaggctttctcatttgcttcgctaccgctattgctcagataaacacggctcataccaggcatcttgctaatcagtttctcgctaaatttgatggtcggcacattaccaataccattggcaaaataacacatttccatcatctgctttgcaacggcatcaacaatttctttacgaccataaccaacattaacggtccaaacaccaccgctaactgcatccagataggttttaccatcaatatcggtgatacgcataccttcacctttcacataaattgccggttcaaagttcttgtgctgggtcagatgatgccaaacatacttcttatccagggcaaccagttctgctttatcataggtcatatgcacctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 35 - DNA, 6637 bp, synthetic construct
  source 1..6637: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatgtcatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcagcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcatgtgcagctcctctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 36 - DNA, 6691 bp, synthetic construct
  source 1..6691: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgcgacccatttgctgtccaccagtcatgctagccatatgtcatcaatgatgatggtgatgatgaccgccaccacccaggccaaatttctgttcaacggtggtaaatgctttatcgatacctgccacaatttcatcgatatctgcttcggttgcaatcagtgccggacacagtgtcagggtattgttaaattcgcgaaagctacggcttgttttaccaatcagaacgcctgcctgttttgctgcaccaaccattgcatttgcaactgcttcggcaatcggttctttggttgcacgatctttaacaatttcaatgcctgcaaacagacctttaccacgaacatcaccaataatcggatgtttcgccatcaggcctttcagaccttccagcagacgatcacccattttggtacagttttccagcaggttttcgcgttcaataatttcaatatttgccagtgctgctgccggaccgctggtacaaccaccaaaggtgctaatatcacgaaaatatgcatcggtatctgccggatcattcacaaaatcctgaaatactttctcggtggtaacggtacagctaatcggtgcataaccgcttgcaacacctttggccatggtaacaatatccggctgcacattgaaatgctgataaccaaaccatttaccggtacgacccagaccacaaacaacttcatcaataatcagcagcagttcatatttgtcgcagatttcacgaatggtttcataataacctgcaggcggaaccagaataccaccgcctgcggtcatcggttcaacaataactgcacccagttcatccggaccaacggtcagaataacttcttccagctgttttgccattttaacacccagatctgcacaatcaccaaatttgctgcggtacacatcacaatccggaaattcatagaaacccggtgcaaacggaccatactgaactttacgttcaaactgaccacatgcgctcagtgtaccaatggtggtgccatgataatcacgtgcacgatacagaataccggtcttcttaccaccatgtttcagctgaccaatctgacgcacgattttaaaggctttctcatttgcttcgctaccgctattgctcagataaacacggctcataccaggcatcttgctaatcagtttctcgctaaatttgatggtcggcacattaccaataccattggcaaaataacacatttccatcatctgctttgcaacggcatcaacaatttctttacgaccataaccaacattaacggtccaaacaccaccgctaactgcatccagataggttttaccatcaatatcggtgatacgcataccttcacctttcacataaattgccggttcaaagttcttgtgctgggtcagatgatgccaaacatacttcttatccagggcaaccagttctgctttatcataggtcatgtgcagctcctctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 37 - DNA, 508 bp, synthetic construct
  source 1..508: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 38 - DNA, 1264 bp, synthetic construct
  source 1..1264: mol_type = other DNA
aagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 39 - DNA, 584 bp, synthetic construct
  source 1..584: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 40 - DNA, 500 bp, synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 41 - DNA, 312 bp, synthetic construct
  source 1..312: mol_type = other DNA
tccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagag

SEQ ID NO: 42 - DNA, 26 bp, synthetic construct
  source 1..26: mol_type = other DNA
gatacaaggctgttagagagataatt

SEQ ID NO: 43 - DNA, 95 bp, synthetic construct
  source 1..95: mol_type = other DNA
cggagtactgtcctccgagcggagtactgtcctccgagcggagtactgtcctccgagcggagtactgtcctccgagcggagtactgtcctccgag

SEQ ID NO: 44 - DNA, 40 bp, synthetic construct
  source 1..40: mol_type = other DNA
tcatatgcacctccttcttaaagttaaacaaaattatttc

SEQ ID NO: 45 - DNA, 30 bp, synthetic construct
  source 1..30: mol_type = other DNA
tgatgagctcactgagatccggctgctaac

SEQ ID NO: 46 - DNA, 29 bp, synthetic construct
  source 1..29: mol_type = other DNA
catcggatcctcctcatcaatgatgatgg

SEQ ID NO: 47 - DNA, 32 bp, synthetic construct
  source 1..32: mol_type = other DNA
taagaaggaggtgcatatgacctatgataaag

SEQ ID NO: 48 - DNA, 32 bp, synthetic construct
  source 1..32: mol_type = other DNA
ggatctcagtgagctcatcagtgatgatgatg

SEQ ID NO: 49 - DNA, 28 bp, synthetic construct
  source 1..28: mol_type = other DNA
ttgatgaggaggatccgatgaatggtgc

SEQ ID NO: 50 - DNA, 20 bp, synthetic construct
  source 1..20: mol_type = other DNA
gaagcttccccttgaccaat

SEQ ID NO: 51 - DNA, 20 bp, synthetic construct
  source 1..20: mol_type = other DNA
ttgattcggataccctgagc

SEQ ID NO: 52 - DNA, 21 bp, synthetic construct
  source 1..21: mol_type = other DNA
gcaaagaaaagccagtatgga

SEQ ID NO: 53 - DNA, 20 bp, synthetic construct
  source 1..20: mol_type = other DNA
tttttcgtctcagccaatcc

SEQ ID NO: 54 - DNA, 43 bp, synthetic construct
  source 1..43: mol_type = other DNA
aaaaaaaaaaaaggcgcgccttccgcattttggacagacgaaa

SEQ ID NO: 55 - DNA, 44 bp, synthetic construct
  source 1..44: mol_type = other DNA
aaaaaaaaaaaaggatccgagatcctttgcgcttttctacgaaa

SEQ ID NO: 56 - DNA, 31 bp, synthetic construct
  source 1..31: mol_type = other DNA
acctcttaccgtgcatatgtcttgagtttag

SEQ ID NO: 57 - DNA, 31 bp, synthetic construct
  source 1..31: mol_type = other DNA
aacgaaatttttcgtagaaaagcgcaaagga

SEQ ID NO: 58 - DNA, 44 bp, synthetic construct
  source 1..44: mol_type = other DNA
aaaaaaaaaaaacatatgtacgtggtaaacccggaagaaaaagt

SEQ ID NO: 59 - DNA, 44 bp, synthetic construct; identical to SEQ ID NO: 55
  source 1..44: mol_type = other DNA

SEQ ID NO: 60 - DNA, 19 bp, synthetic construct
  source 1..19: mol_type = other DNA
gcacccaggcacattgttc

SEQ ID NO: 61 - DNA, 20 bp, synthetic construct
  source 1..20: mol_type = other DNA
tactggggtttctgggagac

SEQ ID NO: 62 - DNA, 20 bp, synthetic construct
  source 1..20: mol_type = other DNA
atgccctctccgtaaatgag

SEQ ID NO: 63 - DNA, 20 bp, synthetic construct
  source 1..20: mol_type = other DNA
gattcaagcacactgcgatc

SEQ ID NO: 64 - protein, 12 aa, synthetic construct
GPCPNEXXGXXD

SEQ ID NO: 65 - DNA, 35 bp, synthetic construct
  source 1..35: mol_type = other DNA
ccttaacaataggcgagtgttaaggggatatcact

SEQ ID NO: 66 - DNA, 10332 bp, synthetic construct
  source 1..10332: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcattgtacacctccttatcaatgatgatggtgatgatgaccgccaccaccaaacttcttcacgtccagggtttctttaaaacgtttggttgcttcttccggactcatcggaatattcggtgcattggtattacccggtttcagaatcacatggataaagctcagttcacgcattgctttgaatttacggaaggcttcaatcacaccttctttgctattaacttttgcggtattctgaatgccgcaggcatttgcaaagatttccagatcgatataacgcagtgcacaggtttcttggctaccggtgctaccgtacgcaccattatccagtgcgataataatcaggttcttcggtgcttcttttgcaatttctaacagtgcattcggattcatcagcaggctaccatcaccatcaaaggtaataacggtcttttcgctacgcagtgctaaacccagaccaatgctgctcacaagacccatgctaccaaacatataaaagttcagctcacgatcacgacatgcatacagttctttacacggaacacccagattggtaacggtaatttcatcatccagttcgcttgccagggtacaaattgcatcattacgcagcataaacggtttcagtgtaccgctggtcaggctaaaacgacaggtacgttccatgatttccggtttaatcggcatacctgctgcctgccatgcacaacattcgcttgcttcccaaactttcgggctaaccagtgcaatatgcggacgggaattttcaaatgcatccagaataacattttccagcagcggcagtttctcggtttcaccaataatggtataggtcagacccgcaccttccagaatgctcggcagatgtgcacccagcggaacctgtgcttcaataccttctttataaacaccacgccagcttgccagaatcggcagcggaattttacaggtcacattcaggctttccagtgcattaatcatattacccagaccggtgctctgaatcagcatcatcggtttaccgcctgccagataaatacctgcacaaataccaacaccattttcttcacgcgtcagtttaatttccgggaagttttcgctaaccagcggcagcagattcttgatacgatcacacggcagggttgctgccagatcaatgccggtctgtttcataatttcgatcactttctcttccgggttcaccacatacatctcgagcctccttatcaatggtgatgatggtggtgaccgccgccgccaacacggctaacggcgtatgcacgcagttcatcaacaacggcttccggcagttccagactttcaccacgttcacgacgcagtgcatgacgacgtgtaccaggcagacgaacaccttcttgttccagcatggcctgaaacagtgcttcgatatgttccagataaccatcactaaaatgtgccggatcaaatgccagcatcagatgaccaacactcggtgcttcaccttccggttcaaagaagctgcttgcctgaaaaccaaaatggctaccggtcagacctgcggtcagcagttcaaccatcagtgccaggcttgcaccttttgcatcaccggcaggaaccatgctacccgcaattgctgcatccggatctgtggtcggacgaccttcaatatctaatgcccaaccttccggaatcggttcaccggccttctttgccagcataactttaccgcgtgcaactttgctcaggctcagatcaataaccagcggatcggtgccacgacgcggtgttgcaaatgcaatcggattggtgccatacagcggacgtttaccaccccacggtgccattgcgctcggtgcattgctaaatgccattgcaaccagaccttcgcgtgccagacgttcaaccggtgcacccgcaacaccaaaatgatggctaccaccaattgcaactgcaactaaacccagttcacgtgccagcggaatggcacgttcaacaccacgtgcaattgccggaaatgccaggccatgacgtgcatcaacacgaataacgctacctgcaacttcaacacgtgcctgggcatctgcaacaactttaccactacgtgcctgtgccagataaaacggcagacggctcagaccatggcttgccagaccatcacgttctgcatcaatcagtgcacgtgcggtaacttcggcttcccaacgcggaacaccaactgcttccagtgctgcaactgccagggtttctgcttcacgcaggctaatcatacggctcatactagtcctccttatcagtggtggtgatgatggtggccgccaccacctttaaagcgcaccactttgcttttgtctttcttgtagatgcccactgcatcaatgctattttccagaatggcgaaggtcacatccttttcatagcccagattcttcagattttctgcgctgctgctattcagaatcaggctcatccaatcatgtttggtcagttccagtgctgctttgataaactcgtcgaactcaccattcagatatttggcaatcacaccgcagccaataaaatcatcgatggcaaagttattttcacgatgacacggaaccaggctaacatcttcaaaatcttccacggcttttgcaacatatttggcgttcacaattgcaccgataaagatgtgttctgcatccaggcttttcagaacacgggtgccattggtggtggtcagaataaccttttcgccgttctcatagcgttctttgataatatctttgttggccagaatttcggtcgggctattaccaaaatcaaaaccctcgattttacgacctttgcgttcaccaatatagatggcgttttctttcttgctggtgctcgtggtaatatacacttcatcaataaagctcagcagggttgtaatggtggtgcttgcacgcagaacatcaacaacaatggcaacattaccacatttatactcggtgaagcgattgcacagggtaatcataaggatcctccttatcaatgatggtggtggtgatgaccaccaccgccaactttaccaaaggtatcaccacgcagaccacgacgcagggtttccaggctgataacttcatcaaatgcaatattggccagattcacactgctaccgaatttcagaataaatgcaacctgctggcttttctgcggtgcttcaaagatcactttgttaatatccacgttcttggccagaacatccagttcgttttctttgactttgccctctttatcaaacaggccaatacctttaccgctttcacgaccttcaataataacataatcggcaccggcatccagatcaaagttaatcagtttgatgcgatcatcaatggtcagctgtttatctttatccggcatcttcttgcccacttcggtcagaaccataaagccgttatctttggcacgtttgattgcattcttacgttcttccaggctaatatcgctgctaccatcactaatttccacggcttcaaaacccagtttctcacattcgttcagaaactcgtcgaatttgcctttgctataggcatattcaaacagggtgccaccaggataaactttgatgccccagtccttatagtagttgattttctctttcacaacatcacgatcaataactgcgctggtgccccaaccaaatttcacaaagtcgatataatctccgcacactttcagataatcctcaacaaatttcggaggcagacctttatccagaacaacggtcagaccacgctgaaaatcttcatacaggaactcaaaggctttcatatgtaacctccttactactaactacgttgccgttaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 67 - DNA, 9118 bp, synthetic construct
  source 1..9118: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcatactagtcctccttatcaatgatgatggtgatgatgaccgccaccaccaaacttcttcacgtccagggtttctttaaaacgtttggttgcttcttccggactcatcggaatattcggtgcattggtattacccggtttcagaatcacatggataaagctcagttcacgcattgctttgaatttacggaaggcttcaatcacaccttctttgctattaacttttgcggtattctgaatgccgcaggcatttgcaaagatttccagatcgatataacgcagtgcacaggtttcttggctaccggtgctaccgtacgcaccattatccagtgcgataataatcaggttcttcggtgcttcttttgcaatttctaacagtgcattcggattcatcagcaggctaccatcaccatcaaaggtaataacggtcttttcgctacgcagtgctaaacccagaccaatgctgctcacaagacccatgctaccaaacatataaaagttcagctcacgatcacgacatgcatacagttctttacacggaacacccagattggtaacggtaatttcatcatccagttcgcttgccagggtacaaattgcatcattacgcagcataaacggtttcagtgtaccgctggtcaggctaaaacgacaggtacgttccatgatttccggtttaatcggcatacctgctgcctgccatgcacaacattcgcttgcttcccaaactttcgggctaaccagtgcaatatgcggacgggaattttcaaatgcatccagaataacattttccagcagcggcagtttctcggtttcaccaataatggtataggtcagacccgcaccttccagaatgctcggcagatgtgcacccagcggaacctgtgcttcaataccttctttataaacaccacgccagcttgccagaatcggcagcggaattttacaggtcacattcaggctttccagtgcattaatcatattacccagaccggtgctctgaatcagcatcatcggtttaccgcctgccagataaatacctgcacaaataccaacaccattttcttcacgcgtcagtttaatttccgggaagttttcgctaaccagcggcagcagattcttgatacgatcacacggcagggttgctgccagatcaatgccggtctgtttcataatttcgatcactttctcttccgggttcaccacatacataaggatcctccttatcaatggtgatgatggtggtgaccgccgccgccggcgcggactaccacctcctgttcagccagccaaccacggacggcgtcacgtacatccccggcaaatgcttcacccgcgcgaacgcgaagatagggctcgacggggtaacgatccaggtcctctgctgcgcgcgcatatccaccgccagtgacgttcaacaaaatatggtcatccgggccaatgaatccttcttcagctgcgcgaatcagagaggcaacagcgacggcggcggctgggtccaggtcaatttcttccgtagcttcgaaaagacgacctgccgaacgggtatcgtcattagcgacagcatacatacgaccacctgatgcagcaagggcgtcgtaaacgccgccacgaacctcccaaggcggatggcggttggtcagaacgtctgccgatacacgtacgatagacgcctctgcatctggcatatccacctcggggacaatttcacgacggccggcttcccaggcacgaaccataggcacaaacggtaagttctgagataagtgcagtgtaggcaagcgcgacccgaagcgtccatctgccactaaacgctcggccgcttcccatgcagcaatgccgcccgtgccagacccaatggcttgaaagtatgcgtcaggcaagcgtccagcgaaaagggctgcatctaacattacggtacccattccgtcgcgacgagcgacgttcttcgcccctccctcgggtacaatacctggaagggaacatacctcacgcccgaaagcgatgctgtcgctgtagtcgccttctaccgtgatcagacaaacgttaggtgctggcaccgtcgtccacaagcgatctgctgcactggcaggtacaactacaactaccggagcacccgtaagggctgaaacttgacagaaggcacggcccgtgtttccggcactgctgatctgaaggacgccggctcccttttcacccaaacgaagtaccgttggttgggcttccagctctttgaaagaacaagtttccatgcgtccaccgcgctccggccaatatccactgaaggtgacggtaaggttagacaaccccaattcacgagccaaaccttcacttgcgtaactcacgggtccggcatcgatgcgtaaatgtccctcgataggaagccaggatgaatagcggaagatgccaggcaagtcgcgcaaggttaagcggtgttccgcataaactgtgcgcagcaaagcattgcatcctaacggacaatccaatgtgtaattatccgggaacaaacgtccacaacctggacaatgaagaaggtacttctcgcgcatatgtaacctccttaaaaagacgcccttaattttctctatagagtatattggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 68 - DNA, 10290 bp, synthetic construct
  source 1..10290: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcagtgatgatgatgatggtgaccaccgccgccttcaaagatttctttgatgatgcccagggctttctcaatatcctcatatttggtggcgtagctaaaacggatatagtttgcaccattttcaccaaatgcaacacccggaacacacagaaccttattttcaatcagcttcttggccacttcaacaccatcaccatattcgctaacatccggaaagatataaaaggcaccatccggtttattcactttgaagatgtctttcaggccgttataaatcagatcacgacgcattttaaactcgcgaaccatatcttcaacacatttctggctaccacgcagtgctgccagtgcaccatactgtgcaaaggtggttgcacatgcaaagctatactggtggatcttgatcatgttgttgatcagatccagttctttgttcagttcatcactaactgccagataaccaatacgccaaccggtcattgcataggttttgctgaagccgttaatcagaatacaacgatcggtaaactgcatcgggctgtagtgtttcttgtcatagatgattttatcgtacacctcgtcgctcacaataatcaggttataatcttccgcaatttcggccaggcctttaatggtttctttatcataaactttaccggtcggattgctcgggctgttaaagatgatcagtttggtcttcttggtgatgctctctttcactttctccaggtcgatgttgaagttctcatccagatcgatattcttgattttgccttcggcaaattcggtcaggctaaaatagctaacaaagctcggattcggaatcagaacttcatcaccacgatcaatcagggtcataatgctcagcatcagtgcttcgcttgcaccacaggtaacaataatgttatctttgtccacatccaggttgtagtcatctttcagtttattgctgatttcttcacgcagttccggaataccattattcgggctataatgggttttaccttcatccagtgcacgttttgctgcttcaataatatgtttcggggtatcgaaatccggttcaccaataccaagattaatacaatcgctggttgccagattaaagatttcgcgaattgcgctcggtttaatgttcttgcagcggctgctaatcatctcgagcctccttatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcatactagtcctccttatcaatgatgatggtgatgatgaccgccaccaccaaacttcttcacgtccagggtttctttaaaacgtttggttgcttcttccggactcatcggaatattcggtgcattggtattacccggtttcagaatcacatggataaagctcagttcacgcattgctttgaatttacggaaggcttcaatcacaccttctttgctattaacttttgcggtattctgaatgccgcaggcatttgcaaagatttccagatcgatataacgcagtgcacaggtttcttggctaccggtgctaccgtacgcaccattatccagtgcgataataatcaggttcttcggtgcttcttttgcaatttctaacagtgcattcggattcatcagcaggctaccatcaccatcaaaggtaataacggtcttttcgctacgcagtgctaaacccagaccaatgctgctcacaagacccatgctaccaaacatataaaagttcagctcacgatcacgacatgcatacagttctttacacggaacacccagattggtaacggtaatttcatcatccagttcgcttgccagggtacaaattgcatcattacgcagcataaacggtttcagtgtaccgctggtcaggctaaaacgacaggtacgttccatgatttccggtttaatcggcatacctgctgcctgccatgcacaacattcgcttgcttcccaaactttcgggctaaccagtgcaatatgcggacgggaattttcaaatgcatccagaataacattttccagcagcggcagtttctcggtttcaccaataatggtataggtcagacccgcaccttccagaatgctcggcagatgtgcacccagcggaacctgtgcttcaataccttctttataaacaccacgccagcttgccagaatcggcagcggaattttacaggtcacattcaggctttccagtgcattaatcatattacccagaccggtgctctgaatcagcatcatcggtttaccgcctgccagataaatacctgcacaaataccaacaccattttcttcacgcgtcagtttaatttccgggaagttttcgctaaccagcggcagcagattcttgatacgatcacacggcagggttgctgccagatcaatgccggtctgtttcataatttcgatcactttctcttccgggttcaccacatacataaggatcctccttatcaatggtgatgatggtggtgaccgccgccgccggcgcggactaccacctcctgttcagccagccaaccacggacggcgtcacgtacatccccggcaaatgcttcacccgcgcgaacgcgaagatagggctcgacggggtaacgatccaggtcctctgctgcgcgcgcatatccaccgccagtgacgttcaacaaaatatggtcatccgggccaatgaatccttcttcagctgcgcgaatcagagaggcaacagcgacggcggcggctgggtccaggtcaatttcttccgtagcttcgaaaagacgacctgccgaacgggtatcgtcattagcgacagcatacatacgaccacctgatgcagcaagggcgtcgtaaacgccgccacgaacctcccaaggcggatggcggttggtcagaacgtctgccgatacacgtacgatagacgcctctgcatctggcatatccacctcggggacaatttcacgacggccggcttcccaggcacgaaccataggcacaaacggtaagttctgagataagtgcagtgtaggcaagcgcgacccgaagcgtccatctgccactaaacgctcggccgcttcccatgcagcaatgccgcccgtgccagacccaatggcttgaaagtatgcgtcaggcaagcgtccagcgaaaagggctgcatctaacattacggtacccattccgtcgcgacgagcgacgttcttcgcccctccctcgggtacaatacctggaagggaacatacctcacgcccgaaagcgatgctgtcgctgtagtcgccttctaccgtgatcagacaaacgttaggtgctggcaccgtcgtccacaagcgatctgctgcactggcaggtacaactacaactaccggagcacccgtaagggctgaaacttgacagaaggcacggcccgtgtttccggcactgctgatctgaaggacgccggctcccttttcacccaaacgaagtaccgttggttgggcttccagctctttgaaagaacaagtttccatgcgtccaccgcgctccggccaatatccactgaaggtgacggtaaggttagacaaccccaattcacgagccaaaccttcacttgcgtaactcacgggtccggcatcgatgcgtaaatgtccctcgataggaagccaggatgaatagcggaagatgccaggcaagtcgcgcaaggttaagcggtgttccgcataaactgtgcgcagcaaagcattgcatcctaacggacaatccaatgtgtaattatccgggaacaaacgtccacaacctggacaatgaagaaggtacttctcgcgcatatgtaacctccttaaaaagacgcccttaattttctctatagagtatattggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 69 - DNA, 7978 bp, synthetic construct; identical to SEQ ID NO: 34
  source 1..7978: mol_type = other DNA

SEQ ID NO: 70 - DNA, 8401 bp, synthetic construct
  source 1..8401: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcataaggatcctccttatcaatgatgatggtgatgatgaccgccacctcctgtataatcacgatatttctccagcaggcgcaccggtttgctcagtgcatcacgacgaaacggatcgcccagttcgcgtgtgcacatgatttcaatcacggtggttttacgatcattcatctgtgcatcaatggctttcttcagcgcaggaccaacatcttcaatacgatcaacaacaacaccttcggcacccatggcacgtgcaatacctgcaaagctctctgattccagttcgcctgcaacaaaacgacggttgtagaaatccacctgattcttcttttctgcaccccactgacgattatgaaacacaactgcggtaaccggaatatcatgacgaacacaggtcataatttcgctcatgctcataccccatgcaccatcaccggcatagctaactgccggacgatgcggtgctgcaacttttgcaccaataatggtcggaaatgcataaccacaattaccccagctcatggctgcaaagaagctacgcggtttctcaaaacgcagataggaattggccacgctattaatgttaccaatatcggtgctaaccataacatcttccggcattgctttctccagttcacgcagaacctgacgcggatgcagccaattgccttcttctttgctctgttcttcaatcatatccaggctaaacggatcacgttcatgggtccaatcggtcagttcttgttcccatgcatctttctctgcctgaatacgtgctgcacgttctgctttatttgcatcacatgccagtttcataccatcaatacgacggctaatttctgctgcgcttgctttggcatcaccacaaacaccaacggtgattttcttcactaaacccagcattttgctgtcggcatcaacctgaataatacgtgcattcttcggccaataatccagaccatgctgaggcagtgtaccaaacggacccagacgggtgcccagtgccagaacaacatctgcatcggccagcagtttcattgctgctttgctaccctgataacccagcggaccacaccacagcggatggcttgccggaaagctatcattatgcagatagctattaaccaccggtgcacccagacgttctgccagtgctttacattcttcaactgcatcacccatcacaacaccaccaccgctgataataaccggaaattttgcttctgccagcagacgggctgctgcatccaggctctgtgcaccaccaggaccacgatccagcggctgcggcagcggaatttcacatttgattttgccgtagaaataatcacgcggaatattcagctgtgcaggacccatttcatcacgtgcacgggcaaaacaacgtgcggtatattcggccatacgtgccggatgggtaacatgaccctgatatttggtgaactcttgaaacatcggcagctgacgtgcttcttgaaaaccacctaaaccaataccggtggtgcctgcttccggtgtaacaatcacaaccggtgtatgtgcccaatatgctgctgcaattgcggtaacacaattgctaatacccggaccattctgaccaataacaacaccggtacgaccgctaacacgtgcaaaaccatctgccatgtgtgctgcaccctgttcatgcacaaccggaatcagtttaatacctgccggtgcaaatatatccatggcatccataaatgcgctacccataataccaaatatttcggtcacaccatttgccaccatggtttcaacaaatgcttcgctcggggtcatttcttgaacaccctgaacaacctgacgggtatcatttgccatatgaaacctccttaaccgtataaatagcttccgcctatctggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccattgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 71 - DNA, 7825 bp, synthetic construct
  source 1..7825: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcaatgatgatggtgatgatgaccgccaccaccaaacttcttcacgtccagggtttctttaaaacgtttggttgcttcttccggactcatcggaatattcggtgcattggtattacccggtttcagaatcacatggataaagctcagttcacgcattgctttgaatttacggaaggcttcaatcacaccttctttgctattaacttttgcggtattctgaatgccgcaggcatttgcaaagatttccagatcgatataacgcagtgcacaggtttcttggctaccggtgctaccgtacgcaccattatccagtgcgataataatcaggttcttcggtgcttcttttgcaatttctaacagtgcattcggattcatcagcaggctaccatcaccatcaaaggtaataacggtcttttcgctacgcagtgctaaacccagaccaatgctgctcacaagacccatgctaccaaacatataaaagttcagctcacgatcacgacatgcatacagttctttacacggaacacccagattggtaacggtaatttcatcatccagttcgcttgccagggtacaaattgcatcattacgcagcataaacggtttcagtgtaccgctggtcaggctaaaacgacaggtacgttccatgatttccggtttaatcggcatacctgctgcctgccatgcacaacattcgcttgcttcccaaactttcgggctaaccagtgcaatatgcggacgggaattttcaaatgcatccagaataacattttccagcagcggcagtttctcggtttcaccaataatggtataggtcagacccgcaccttccagaatgctcggcagatgtgcacccagcggaacctgtgcttcaataccttctttataaacaccacgccagcttgccagaatcggcagcggaattttacaggtcacattcaggctttccagtgcattaatcatattacccagaccggtgctctgaatcagcatcatcggtttaccgcctgccagataaatacctgcacaaataccaacaccattttcttcacgcgtcagtttaatttccgggaagttttcgctaaccagcggcagcagattcttgatacgatcacacggcagggttgctgccagatcaatgccggtctgtttcataatttcgatcactttctcttccgggttcaccacatacataaggatcctccttatcaatgatgatggtgatgatgaccgccaccacccaggccaaatttctgttcaacggtggtaaatgctttatcgatacctgccacaatttcatcgatatctgcttcggttgcaatcagtgccggacacagtgtcagggtattgttaaattcgcgaaagctacggcttgttttaccaatcagaacgcctgcctgttttgctgcaccaaccattgcatttgcaactgcttcggcaatcggttctttggttgcacgatctttaacaatttcaatgcctgcaaacagacctttaccacgaacatcaccaataatcggatgtttcgccatcaggcctttcagaccttccagcagacgatcacccattttggtacagttttccagcaggttttcgcgttcaataatttcaatatttgccagtgctgctgccggaccgctggtacaaccaccaaaggtgctaatatcacgaaaatatgcatcggtatctgccggatcattcacaaaatcctgaaatactttctcggtggtaacggtacagctaatcggtgcataaccgcttgcaacacctttggccatggtaacaatatccggctgcacattgaaatgctgataaccaaaccatttaccggtacgacccagaccacaaacaacttcatcaataatcagcagcagttcatatttgtcgcagatttcacgaatggtttcataataacctgcaggcggaaccagaataccaccgcctgcggtcatcggttcaacaataactgcacccagttcatccggaccaacggtcagaataacttcttccagctgttttgccattttaacacccagatctgcacaatcaccaaatttgctgcggtacacatcacaatccggaaattcatagaaacccggtgcaaacggaccatactgaactttacgttcaaactgaccacatgcgctcagtgtaccaatggtggtgccatgataatcacgtgcacgatacagaataccggtcttcttaccaccatgtttcagctgaccaatctgacgcacgattttaaaggctttctcatttgcttcgctaccgctattgctcagataaacacggctcataccaggcatcttgctaatcagtttctcgctaaatttgatggtcggcacattaccaataccattggcaaaataacacatttccatcatctgctttgcaacggcatcaacaatttctttacgaccataaccaacattaacggtccaaacaccaccgctaactgcatccagataggttttaccatcaatatcggtgatacgcataccttcacctttcacataaattgccggttcaaagttcttgtgctgggtcagatgatgccaaacatacttcttatccagggcaaccagttctgctttatcataggtcatatgaaaacctccttattatgggtcgtaatcgacttgctaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 72 - DNA, 13065 bp, synthetic construct
  source 1..13065: mol_type = other DNA
atccggatatagttcctcctttcagcaaaaaacccctcaagacccgtttagaggccccaaggggttatgctagttattgctcagcggtggcagcagccaactcagcttcctttcgggctttgttagcagccggatctcagtaagcttatcagtggtggtgatgatggtggccgccaccacctttcaccaggttatatgcccattcaatccattcacacagacactgcagatcttctttctcaacggttgcaccacaccaaatacgcagaccgctcggtgcatcacgatagctaccaatatcataggccaccttttctttctccagggttttaatcagctctttcagcttttcgtcactcagatcaactttaaagcaaacgctggtgctgctacgaatttctttggtttcggccagaaaatgaatccagttattcttggccacaaatgcttcaaacactgccagattatcattggtacgctgaatcagcggtttcagaccaccaacgctttctgcccatttcagggttgccagccaatcttcatttgccagcatgctcggggtattaatggtgctgccttcaaagatcttcttgttcagtttgccacctttggtcagacgaaagattttcggcagaggccatgccggtgtatagctttccagacgctgaactgcacgcggactcagaatcagcataccatgtgcaccttcaccacctaaaactttctgccagctaaaggtaatcacatccagtttatgatacgggatatccattgcaaagattgcgctggttgcatcacacagggtcagaccttcacgattttccggaatccaatcaccattcggaactttaacaccgctggttgtaccattccacacaaacaccacatcgttcttgaaatccaccttcttcagatccggcagtttaccatattcggcttcgaagacacgcacatccttcagtttcagctgtttggtaatatcggttgcccaacctttgctaaaactttcccaaaccagaacatcaacaccacgacaacccagcattgaccacagacacatttcaaatgcaccggtatcgcttgccggaacaataccaaccagataatcatccggcagacccagcatatcacgggtcttcttgatggcttctgccagtttctctttacccagattgctacgatggctacgaccaaacggggtatctttcagttcttcaatgctataacccggatgttttgcacacggaccgctgctaaaacacggattattcggaacacgtgtcggtttcatgagctcctccttatcaatgatggtgatggtgatgaccaccaccgcccagattaatcacggccacgtctttgatgttcgggatttctttaatcttttcaatcacttcttccggcacggtgtgatccagattcagcagcatcacgctttcaccaccaggttctttacgaccaacctgcatgcttgcaatgttaataccataatcgcccagggtaatacaaacacggccaattgtacccggacgatcaatatgtttgataatggccagaacaccttccggaataaagctaacttcataaccatccacttccagaataaccggtttgttattgatgatggcacccacaatgctgaatttcttcttatcgctctctgcggtaattttgatggcgttaccatatttctcttcgctggtcgagctttcgacaacattgatattacggttcttggcaataatcggtgcattcaccagattaatacctgccagcagaatcgggctcagcagacctttcagaaatgcgcgtttaatcagatcggtcttttcttttgccagttcgcctgaataaatcagttccacacgattaacgctaccatccagaacctgcataacaatattacccagcatttcggccagcagcatatacggtttcagtttacccagtttctcttgcggaatgttcggcatattcacaacattttctgccagttcaccacgcagaactttcttgatctgctctgcaacaatggtgcctgctgctttctgtgcttcttcggtgcttgcaccctgatgcggtgtaccaataacattatccagggtcagcagcggattatctttaggcggttcttcttcaaagacatccagggctgctgcgcgaattttaccttctttcagtgcttcatacagtgctttctcatcgatcagaccaccgcgtgcacaattcacaataatggcgttcttcttcatcagtgcaatctgctcgcgaccaatgatatgacgtgttttcggtgtcagcggaacatgcagggtgataaaatcggcacgtttacacagttcattaatatcatccaccagttcaacacccatgctttctgcaacttctttcggaatatacggatcataaccaatgatgttcatgccaaatgcttttgcacgtttaacaacctgctgaccaatacgacccagaccaatcacacccagggttttaccatacagctcaatgcctttaaaacgtttacgatcccattcaccacgtttcaggcttgcggttgcctgcggaatattacgtgctgctgccagcatcagacccatggtcagttctgcaacgctaatgctgcttgcatccggtgcattaacaacaataatgcccttctcggtggctgcttcaacatcaatattatcaacaccaacaccggcacgaccaataactttcagtttctcggccttctcaataacatcacgggtaactttggtgccgctacgaacaaccagaacatcggcatctttgatcttctccagcagttcttctttggtcagaccggttgcaacttcaacttcgccaacttcttccaggattttgattgcatcttcatgcagcggatcggtaaccagaattttcaccattgtacacctccttatcagtgatgatgatgatggtgaccaccgccgccttcaaagatttctttgatgatgcccagggctttctcaatatcctcatatttggtggcgtagctaaaacggatatagtttgcaccattttcaccaaatgcaacacccggaacacacagaaccttattttcaatcagcttcttggccacttcaacaccatcaccatattcgctaacatccggaaagatataaaaggcaccatccggtttattcactttgaagatgtctttcaggccgttataaatcagatcacgacgcattttaaactcgcgaaccatatcttcaacacatttctggctaccacgcagtgctgccagtgcaccatactgtgcaaaggtggttgcacatgcaaagctatactggtggatcttgatcatgttgttgatcagatccagttctttgttcagttcatcactaactgccagataaccaatacgccaaccggtcattgcataggttttgctgaagccgttaatcagaatacaacgatcggtaaactgcatcgggctgtagtgtttcttgtcatagatgattttatcgtacacctcgtcgctcacaataatcaggttataatcttccgcaatttcggccaggcctttaatggtttctttatcataaactttaccggtcggattgctcgggctgttaaagatgatcagtttggtcttcttggtgatgctctctttcactttctccaggtcgatgttgaagttctcatccagatcgatattcttgattttgccttcggcaaattcggtcaggctaaaatagctaacaaagctcggattcggaatcagaacttcatcaccacgatcaatcagggtcataatgctcagcatcagtgcttcgcttgcaccacaggtaacaataatgttatctttgtccacatccaggttgtagtcatctttcagtttattgctgatttcttcacgcagttccggaataccattattcgggctataatgggttttaccttcatccagtgcacgttttgctgcttcaataatatgtttcggggtatcgaaatccggttcaccaataccaagattaatacaatcgctggttgccagattaaagatttcgcgaattgcgctcggtttaatgttcttgcagcggctgctaatcatctcgagcctccttatcagtgatgatgatggtgatgaccaccgccaccttcacgccagctaattttctcaaccggttctttcagacgaaatttaccgctcagcacctgtgctttcagttcttctgctgctttaacggcacgtgcacgatcgctctgacgcagggtaactttaatatcgcgaaagcaaccaccaactgcacagctcactgtacccagttttgcgctaaatgcttcacctttacagcggctaatacacagaccacaattaaaacacagttccggattatgaaccgcaccattttcacctttgctaactgcacccatcggacatgcttcggcaacacaacaaattttgcaatcgatgcatttctccggctcatatttaacgctcaggtctgtattatcccaaacatcaccataggtaatttcacacagcggcagacgacctgccagatcaaccagtttcagcggaatctggctatccagctgcagaatattttccagcatttcacggctcagaaccggaatcggaactgcccaggtattaacaatttccggacctgcgctggtcacaaaaccacccatatattccggtgtcatatcatgcagatcggcaaaaccggtcagattcgggttatccggtgctgaacgggtgccctgaccggtaacaaaaccttctgcaccattaatcagaacacgggtgccaataccaatggtttccagtttcggatcattctgaatcgggttcagttcaccacaaccacaaaaggtcagttcacgaaattcaccttcaaacggcagggcatgaaatatggttttgatgctttctttacccggattcacaaatgcacgatagttcttaaaggcatgacgggttgcatacagacgtgcaaacggcatttcgctcagacgggtttcaacgctgaagaaatcaccaactgaggtggtcacatcaacggtaatgcttttaccttcaaccatatcacgaaacaggtgaccaccaccataacggctatcggtttcgctatgtgcggtgcccagaacaatcagatccagaacacccagacgttcattcggacacggaccaacaattgccggaacaccattcagcagaactttgcttgctttaacaaagctatccggttcgctaactttaaagctaaaaactgcataggtgccgctcataatgccacgtgttgcggtggtaacaacatcaatatcttccagacgaatttcaccaccggcacgaacttttgcaccaatttcttcggctgtcagaacaactgcttcaccggcatcaattttaacctggatttcttgaatggtacgcggtttctcggtattatccagtgcaccattcatactagtcctccttatcaatgatgatggtgatgatgaccgccaccaccaaacttcttcacgtccagggtttctttaaaacgtttggttgcttcttccggactcatcggaatattcggtgcattggtattacccggtttcagaatcacatggataaagctcagttcacgcattgctttgaatttacggaaggcttcaatcacaccttctttgctattaacttttgcggtattctgaatgccgcaggcatttgcaaagatttccagatcgatataacgcagtgcacaggtttcttggctaccggtgctaccgtacgcaccattatccagtgcgataataatcaggttcttcggtgcttcttttgcaatttctaacagtgcattcggattcatcagcaggctaccatcaccatcaaaggtaataacggtcttttcgctacgcagtgctaaacccagaccaatgctgctcacaagacccatgctaccaaacatataaaagttcagctcacgatcacgacatgcatacagttctttacacggaacacccagattggtaacggtaatttcatcatccagttcgcttgccagggtacaaattgcatcattacgcagcataaacggtttcagtgtaccgctggtcaggctaaaacgacaggtacgttccatgatttccggtttaatcggcatacctgctgcctgccatgcacaacattcgcttgcttcccaaactttcgggctaaccagtgcaatatgcggacgggaattttcaaatgcatccagaataacattttccagcagcggcagtttctcggtttcaccaataatggtataggtcagacccgcaccttccagaatgctcggcagatgtgcacccagcggaacctgtgcttcaataccttctttataaacaccacgccagcttgccagaatcggcagcggaattttacaggtcacattcaggctttccagtgcattaatcatattacccagaccggtgctctgaatcagcatcatcggtttaccgcctgccagataaatacctgcacaaataccaacaccattttcttcacgcgtcagtttaatttccgggaagttttcgctaaccagcggcagcagattcttgatacgatcacacggcagggttgctgccagatcaatgccggtctgtttcataatttcgatcactttctcttccgggttcaccacatacataaggatcctccttatcaatggtgatgatggtggtgaccgccgccgccggcgcggactaccacctcctgttcagccagccaaccacggacggcgtcacgtacatccccggcaaatgcttcacccgcgcgaacgcgaagatagggctcgacggggtaacgatccaggtcctctgctgcgcgcgcatatccaccgccagtgacgttcaacaaaatatggtcatccgggccaatgaatccttcttcagctgcgcgaatcagagaggcaacagcgacggcggcggctgggtccaggtcaatttcttccgtagcttcgaaaagacgacctgccgaacgggtatcgtcattagcgacagcatacatacgaccacctgatgcagcaagggcgtcgtaaacgccgccacgaacctcccaaggcggatggcggttggtcagaacgtctgccgatacacgtacgatagacgcctctgcatctggcatatccacctcggggacaatttcacgacggccggcttcccaggcacgaaccataggcacaaacggtaagttctgagataagtgcagtgtaggcaagcgcgacccgaagcgtccatctgccactaaacgctcggccgcttcccatgcagcaatgccgcccgtgccagacccaatggcttgaaagtatgcgtcaggcaagcgtccagcgaaaagggctgcatctaacattacggtacccattccgtcgcgacgagcgacgttcttcgcccctccctcgggtacaatacctggaagggaacatacctcacgcccgaaagcgatgctgtcgctgtagtcgccttctaccgtgatcagacaaacgttaggtgctggcaccgtcgtccacaagcgatctgctgcactggcaggtacaactacaactaccggagcacccgtaagggctgaaacttgacagaaggcacggcccgtgtttccggcactgctgatctgaaggacgccggctcccttttcacccaaacgaagtaccgttggttgggcttccagctctttgaaagaacaagtttccatgcgtccaccgcgctccggccaatatccactgaaggtgacggtaaggttagacaaccccaattcacgagccaaaccttcacttgcgtaactcacgggtccggcatcgatgcgtaaatgtccctcgataggaagccaggatgaatagcggaagatgccaggcaagtcgcgcaaggttaagcggtgttccgcataaactgtgcgcagcaaagcattgcatcctaacggacaatccaatgtgtaattatccgggaacaaacgtccacaacctggacaatgaagaaggtacttctcgcgcatatgtaacctccttaaaaagacgcccttaattttctctatagagtatattggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 73 - DNA, 34 bp, synthetic construct
  source 1..34: mol_type = other DNA
cctaacggcaacgtagttagtagtaaggaggtta

SEQ ID NO: 74 - DNA, 48 bp, synthetic construct
  source 1..48: mol_type = other DNA
ccaatatactctatagagaaaattaagggcgtctttttaaggaggtta

SEQ ID NO: 75 - DNA, 13 bp, synthetic construct
  source 1..13: mol_type = other DNA
taagaaggaggtg

SEQ ID NO: 76 - DNA, 42 bp, synthetic construct
  source 1..42: mol_type = other DNA
ccagataggcggaagctatttatacggttaaggaggtttcat

SEQ ID NO: 77 - DNA, 38 bp, synthetic construct
  source 1..38: mol_type = other DNA
cctagcaagtcgattacgacccataataaggaggtttt

SEQ ID NO: 78 - protein, 438 aa, Methanosarcina mazei
MNGASANIEEHRTILEIQAKIDAGEAVVFTAEEISSRIRAGEEIGLEDVDVVTTATRGIMSGTYAVFSFKVSEPDSFIKASEVFLNGVPAVVGPCPNERLGVLDIIVLGTAHSVSDPRYGGGHLFRDMVEGKIIRVDVTTNEGKCFSVETCLSEIPFARLYATRHAFKNYRAFVNPGKDPIDTIFHALPFEGEFREMTFCGCGELNPIENVPGLETIGIGTRVLINGAEGFVTGQGTRSVPDNPNLTGFADLHDMVPEYMGGFVTSGGPEIINTWAVPIPVLSRSMLENILKLDSQIPLKLVDLAGRIPLCDITYGDVWDNADLRVKYDPGKCINCKVCPVAEACPMGAVSKGENGAEHNPELCFNCGLCISRCRGEAFRANLGTVRCEAGGCLRDIKVTLRQSDRARAVKAAQELKEQLLTGRFRLTEPVEKISWRE

SEQ ID NO: 79 - protein, 438 aa, Methanosarcina barkeri
MNGELDNTETSRTIPEIQDKIDAGDAIVLTAAEISEKIRAGEDIKLEDVDVVTTATRGIMSGTYAVLSFKVSEPDSFVKASEVLLNGVPAVVGPCPNERLGILDLIVLGTAHSKFDPNYGGGHLFREMVEGKNIKVDVTTSEGNHFSVETRLSEIPYAKLNATRHAFKNYRAFVNPGKESIKTIFHALPFEGEFKEMTFCGCGELNPIENDPKLETIGIGTRVLLNGADGFVTGAGTRSAPDNPNLTGFADMHEMTPEYMGGFITSAGPEIINTWAVPIPILHEGILENILKLDKEIPLKLVDLAGRIPLCEITYGDVWDNVDLNIKYDPEKCLNCKDCLVIEACPMGAVSRRENGAVHNPEFCFNCGLCISRCRGEAFSANLGSVRCSTGGCLRDIKVTLRQSDRARAIIAAEELKEKILTGRFRLNEPVEKISWRE

SEQ ID NO: 80 - protein, 431 aa, Methanohalobium evestigatum
MSDKERTYQEIQRKIDNGKAVVLTADEICNRVRSGEKINFEDVDVVTTATRGIMSGTYAVFSFKVAEPNSFIKASRVWLNNVPAQVGPCPNERLGVLDLIVHGTAHSKTNPNYGGGHLFREIVEGKPITVEIETVEGHWFSTHTNIKDMQYAKLFGTRHAFRNYLAFVNPVNESLPSIFHAAEFRGNLSEATFCGCGELNPIENDPDLQTIGIGTRVMINGNYGYVSGLGTRSSPGKPNLGGFSDMHEMIPEYMGGFVTSAGPEIINTWAVPIPVINQDILDNIVKINSDIELKVVDVSGRLPLCKTNYADVWSNVDEEVTYQPSECIDCRVCEVERTCPMGAVTHKRGESAAHNPILCFNCGLCTTNCAGNAFSAALGNIDYCTAETEKGIPVVVRQSDRLRALKAADELKTKIISGKFKISEPVEYINL

SEQ ID NO: 81 - protein, 426 aa, Methanohalophilus mahii
MERAVGHILEKINRKDATVLTSQELCDMIDAGDNVGFEDVEVVTAATRAIMSGTYAVLSFPVDTVEKFTRACTVTINGVDAHVGPCPNERLGILDLMVFGTAHSHERENYGGGHLFREIVEGNPVDVDVVTDEGRKYSDTVSIEDMPYAQLFATRHAFKNYSAFVNFSPSPVSTIFHATAFAPDATEATLSGCGQINPVKNDPQLHGIGVGSRILLNGAEGFILGSGTRSSPEKPNLIATADMHSMVADHMGGFATSAGPECIVSWAVAVPVVDDSVFDAIRQTDSAVPMPVMDADRRIKVAMTSYADAWKDVDLEVAFNPGNCRGCDVCEPMKRCPMESIYFNGDKMVLNRHTCFNCGLCSTLCSSVFKAELGSLKFDYEGKNMDVPIVVRQSDRKRALIMAEKLRDSIKNGSFILNGMVERISP

SEQ ID NO: 82 - protein, 427 aa, Methanococcoides burtonii
MEQSIEQINRKIEKGDAVVLTAQQVCDLVDSGEDIRAEDVDVVTAATRAIMSGTYAILSFPVKSSHSFLRAKEVYMNGVPAHVGPCPNERLGILDLMLFGTDHSVDDSRYGAGHLFRDLAERMTVEVKVITDNGESFKTQVTLDGMPYAMLYGTRHAFKNYSAFVNTSDEPAASIFHAMEFGPRLTEATISGCGQINPVKNDPLLESIGIGTRMLMNGSEGFIMGSGTRSSAEKPNLTAFADMHEMKPEYMGGFSTSAGPECIVSWAVPVPVTSDSVLESIKERDRQLKMPVMAVDNRACVGYSDYGDVWEGTDLAIDFSPDECIGCTLCEPAEMCPMASISFGDGEVKLDRYTCFNCGLCSTLCVTDVFTANLGSINFELGGESTNVPIVLRQSDKKRALELSEKLKEQILDGSFRMTSMVERIRT

SEQ ID NO: 83 - protein, 420 aa, Methanocella arvoryzae
MVTTKTIPEINERIEKGTVTVMTATEILAIAEKGERIPDVDVVTCGTKGIMSGTMASLSFKVAEKNAFIRAKGLWLNGVPAYPGPCPNERLGIVDAIVYGTAHSTYDHHYGGGHLFRDLVEGNEIEVEVDTGLAERLTTTVTLSDMGHARMIATRHAFKNYLAYVNPVAETLEKSIFSVGAFYGPYKELKFCGCGELNPIEKDPDLEVLGAGTRALINGAPGMITGAGTRSTREKPNFSAVADLKQMKSEYLGGFVTSEGPEVIASWAVPVPVLNDRVLKNILKTDKEIPLPVVDVRGRIELGRITYADVWKNCDFAVRFNPDLCDSCEPCKGELICPTAALSKESKRIDRSRCFNCGTCVGLCPAVSANMGSVEFNGMTIPITERHSDRLGAMKISNELKKQIASGEFKLSEIVQRITF

SEQ ID NO: 84 - protein, 414 aa, Methylocella palustris
MKTIDEINQKIREGTAVVLTADELKARIREGETVTPEKVDVVTTGTFGVMSGTMAVLSFSVAKPGTFQKAEQVLLNGVPAYPGPCPNERNGILDLVVYGTAHRDHRYGGGHLFRDIVAGHVIAVEVVAGEQTLETMITLNQMGLARIIATRMAFRNYHAMINRSVDPIQTIFSVTGLAGPCTEVSVSGCGEINPLENDPERRFIGTGTRVLLNGGTGYVIGEGTRSSQEKPNLSVYGDMKGMNPQMMGGFITSVGPECLTSLAAPIPVLDQAALDPLLVLDAEVNLPIAGINDRTGCAASKYSEVWQGTDHRIQNDPSGCVHCETCRAATLCPTGAISPGAVIDRDRCVNCGTCVQVCPGPAFSGILGAITYEGRQVPITLRQSDRIRGTRLCAELKERIRVGTFSLTRKVDSL

SEQ ID NO: 85 - protein, 413 aa, Methanoculleus marisnigri
MKTIAEIDEKIRNGSAVVYTAAEFKRLIREGADITAAEVDVVTTGTSGVMSGTAAILSVPVAEPGTFERAERAWLNGVPCMPGPCPNERLGILDLIVSGTAHAGASYGGGHLFREIVEGREIEVVVEAADRSIEARVTLDDFSYARLFTTRSAFRNYTAYVNTKPTRMKTIFSVTGLQGPCREVSVSGCGEINPLQNDPARLAIRDGTPVFLNGSAGIVTGEGTRSTPSRPNLTVIADMAGMQPRYMGGFATSAGPECITSLGVAIPVLDDRQVAALRVLDEEIPLPVADINTRTVLGAGTYADVWQRPDREVAYYPEWCEGCSACAAAVICPTGAFTRETGIDRDRCLGCTACMASCPNNALEAGEGSIRVRGRRVPITLRQSGRTLAEDLCRDVKERILDARFTFTGGGGR

SEQ ID NO: 86 - protein, 408 aa, Methanocorpusculum labreanum
MKTLAEINRKIADKTAVVITAKSLKERIRKGEIITPDDVDVVTCGTFGVMSGTSAFLAFQAGTPGTFRHVVSMTLNGVPAFIGPCPNESNGHVDCIIYGTSRAEQNPSYGGGHLFSDLAAGKPVTAEIITDTGTIRRLVSVDDMSAARLIVTRGGFKNYMGFVNRSEEEISTIFSTFPMKPKMQSASVSGCGEINPLENDPKLRYHVPGASALVNGSPALILGCGTRSSPEKPNLSLSADMQGMHPDMMGGFVTALGAECLTSIGTAIPVLDDETMDALRILDEDIPLPVADIQNRTPFAAATYADVWQNTDGRVRVDASRCVNECTECAKDLCPVAAIRPDLSIGKACMGCLTCITVCRHQVFSANAGTLHIPDADIRIGLRQSDRVRGNRAAALVRDRISSGAWRF

SEQ ID NO: 87 - protein, 412 aa, Methanolacinia petrolearia
MVKTVAEINKKIRDGTAVVLTAGEFKKKVRDGERLSPDDIDVVTCGTCGVMSGTMAILSVPVAEPGTFRRADSITLNGVPAFPGPCPNEGLGQVDLVVYGTSHASNSYGGGNLFRDIASAEKEIEVQTEAGGKNFECTVYGDELHYARMITSRSAFKNYSAFVNSGKDPVNTIFSVLPLSGGLTQATVSGCGEINPLQNDPDTRFLRSGAGVLLNGAEGLILGTGTRSSSEKPNLSVSAGMAGMNPEFMGEFKTSAGPECITSIAAAIPVIDNECIARLSVLDEEILLPIVDVFDRRTLGFSDYGRIWQSAGRHITVDPTKCLLCGECPAQEHCPADAIMPGGGILTSRCLSCGACLSTCIGEVYSMDTGSIALNGKEIPVVLRQSDRRKGEQICSNLKEKIESGKFTLGGM

SEQ ID NO: 88 - protein, 405 aa, Methanospirillum hungatei
MRSIDEIRTALADGSAVILTASELKKRITQGDTVSDVDVVTCGTCGVMSGTYAVLSVPVAPPGTFSRADTISLNGVPAIPGPCPNERLGLVDLMVFGTAHADDRYGGGHLFHDLIAGEEIHIAVTSAGKDYEAYVTLSDLPFARLFTTRSAFKNYSAIINQSSGPVKTIFSALPLQGTSTQATVSGCGEINPVENDPTLRFLSAGSPVILNGAGGRVIGTGTRSSPYRPNLSVHADLKEMDAHFCGGFRTAAGPECITSIGTAIPVVDTDVLSSLSVLDCDIAMPVVDISDRQPVGTASYEQVWTGTATRVLYHPERCLHCSPCDARTACPVDAIREDGTINQTQCFVCGTCVQICKGSVYEGNFGSITYEGAGIPIVLRQSDRRRAEILCRRVRDMLVRGEFQI

SEQ ID NO: 89 - protein, 431 aa, Methanothermobacter thermautotrophicus
MSLWPNRSRNWRVHCDCRGFNLDRHVSEINERILEGDAAVFTADEIKDMVRDGDVPSADEVDVVTTATCGVMSGTAAVMHLRVSEPGSFRKAASVELNGIPAYPGPCPNENLGSVDLFIYGTGHSREDPDYGGGFLFRDLLLGSEVEVRVTSVEGRVVESTVTMDDIETARIVGTRMAFRNYTALVNPGETPVKSIFNAFEMPENCGGLSFSGCGDINPLENDPSMETVHQGTGVLLNGARAIVLGEGTRSSPIKPNLMLSGDLHDMKPRYIGGFRTAAGPEIFNTVAVPVPVTSERVLENLMVLNSDIKLPVADVRDRSRVITEITYEDVWSGDVRPVHHPERCTDCAVCLAARRCPTHAIDNGLDLDRCFGCGVCAWSCPSGAYEMDTGTVRIGELAVPIICRQSDRLRARELSLELKKLIENGDFLMG

SEQ ID NO: 90 - protein, 412 aa, Methanothermobacter marburgensis
MRRTISEINERILDGEATVLTAGELKELVLNDEAPSADEVDVVTAATCGVMSGTAAVMHFRVSEPGSFRKAVSAELNGIPAYPGPCPNENLGSVDLFIYGTARSVRDPEYGGGFLLRDLLIGSEVDVTVKSEDGTSIESTVTLKDIETARIIGTRMAFRNYTAIVNPSDKPVRSIFNAFEMPPHCGGLSFSGCGDINPLENDPGMEAIRRGTSVLLNGARGLVLGEGTRSSPERPNLMLSGDLHDMKPDYIGGFRTAAGPEIFNTIAVPLPVTSERVFERLLVLNSDIGLPVADVRDRSRIIHDLTYADTWAGDERPTYNPERCADCLPCLAAERCPTHAIGDGLDTDRCFGCGVCAFSCPYGAYEMDTGEVRIGDRTVPIVCRQSDRVRASSLARELKELIEKGNFLIGGC

SEQ ID NO: 91 - protein, 408 aa, Methanothermus fervidus
MNSDKLKSISEINEKIEKGDVQVFTASEIKKMSKVSVEDVDVVTTGTCGIMSGTSAIFHFKISEPGAFRKVKEIYLNGVPGFPGPCPNENLGSVDLIVYGTSKSIYDHNYGGGFLFKDIIEGKNIQIEAKTVEGRKIETEINIEDMETSKMLGTRMAFQNYSAFLNSKKGIKKTIFSVTGLRGPFKELSFSGCGELNPLQNDPEMYVINVGSKALLNGSEAIILGLGTRSSKKPNLMIMAEMKDMESYYIGGFKTGSGPEIFNTIAIPIPILNKKILNNVLIKNSKIPLPILDVNDRREIGKITYADVWNGVDKRPKYKKDKCLKCKECKIEKICPTDAFKPKEGIDFKKCFGCSVCAHHCKAIEMKTGVVEFNSAKIPVTCRQSDIKRAEKLSLELKKRIENGEFYL

SEQ ID NO: 92 - protein, 415 aa, Methanobrevibacter smithii
MKERTIGQINKKIENNEAKIYTAEEFKKLIKEDNAPSFEEVDVVTAGTCGVMSGTAAIFNFTVSEPGVFMRAKNIYLNGVPANIGPCPNEWLGSVDLILHGTSKSVEDSSYGGGFLLKDILEGKDIDVKVEAINGDIIESTTNIGEITRAQMIGTRMAFKNYTAFVNPSAEQVSSIFSAIPLEGNLKGLTFSGCGDLNPLQNDPNNLIIGNGTKVLLNGAEAIVLGEGTRSSPEKPNLMVSGNLKDMEANYVGGFKTGEGGEVYDTLAIPIPVLNEEIYNNLLIKNSDIPLTVADIKGRHLPLTQTDYGAMWDNHELRPKYVSDNCGCDSCTVEDVCPTNAFKNKRLDLSKCFGCGMCVHYCKNNSFTMDAGNVDLEIGGKKHNIPIICRQSDALRANKLSLKLKKMIKNHEFKL

SEQ ID NO: 93 - protein, 418 aa, Methanobacterium formicicum
MKKSIPDINQKIKNGEATILTAEEVTRLVMDGEEPTVEDVDVVTTGTCGIMSGTAAIFHLPVAEPGSFKKSRKITLNGVPGFPGPCPNEWLGSVDLMVYGTSHSITDPQYGGGFLFKDLLRGEEIEIDVEDIDGNILKSSATLDDFGTAQMIGTRFAFKNYTAFINPTSQPVSSIFNAVDMDGPFKGISFSGCGELNPLQNDPQLNSIQKGTRLLINNSEGLFIGPGTRSSPEKPNMMITADMKDMDPHYLGGFRTGAGPEVYNSVATAIPILDDEILRKTFIKNEDIRLPVADIRGRHSVLSQTTYQVWRNVDERPTYEKELCQNCQTCMVEERCPTRAFQNQTLNPVRCFGCGMCAYSCPFGTFQMERGQVQIDWEGKVKELEVSCRQSDIKRARELARELKNRIERGEFLLNNNL

SEQ ID NO: 94 - protein, 380 aa, Methanocaldococcus jannaschii
MEKELKVITIDELKKYIRNNEEDKIEEVDVVTSATCGIMSGTAGIFHIPFNEVFKRAEEIYLNDIKGVVGICPNEFLGKVDAIFYGEVGFLFKDLVKGKVVEAKAISEGKIYKNEITIDDLPTAKMIGTRMAFKNYTAITNLSDEEVNTIFHRLPLKKGEASFSGCGMLNPLENMVIKDEKDVVGKKALLNGAEAIILGFGTRASIEKPNLMMSADMKDMDAYYLGGFVTSNGIEIYNTIAVPIKVDEHKEALKKLDKDITLPLVNIFGREIIDIGSYAEVWENVDLRPKIYQDKCKNCRECLVEKYCPTFAIKRENGKIKITEDCFGCGVCNICPYGVFKTKLGSVCGIPITCRQSDRKRALKLAKELKKKIERGEFKI

SEQ ID NO: 95 - protein, 394 aa, Methanococcus voltae
MENCEKIIVTVEELKKMVKNNEEDKINEVDVVTCGTCGIMSGTMAIMHIPMGQKFRKAEKFTLNGINCNVGPCPNEYLGSVDITVNGTTHTEDYGGGFLFKDLIAGKEVVAKAYCDNVVYEKTLTLSDIPTAELIGTRMAFKNYVALTNLGEPVNTIFHRVALKKGEASFSGCGEYNPLQNMETEYSNLFGKKILLNGSEGLILGYGTRYSAEKPNLMISADMHNMDDYYIGGFHTSAGAEIYDTVAVPIEMNDKNKEYLMTIDDDIVLPLTNVLGRELIKKASYGEAWNNTDLRPKVDLTKCTENKHRDGIIPLSKFCEAVALCPTKALENGIPSKDCFGCGVCATVCHEGVYSMNMNTIGGIPITCRQSDRVRALALSKELKKRIEKGEFKL

SEQ ID NO: 96 - protein, 388 aa, Methanococcus maripaludis
MEKLVVTVDELKKMVRNNEEDKIDEVDVITAGTCGIMSGTLGIFHIPLGESFKKAEKFYLNGIEGYPGPCPNEFLGSVDCTIYGTKHFNNYGGGFLFKDLISGKEVEAVVESNGKTYKKMITLKDMPTARLIGTRNAFKNYVALTNTGEPVNTIFHRKMLKSGEASFSGCGELNPLQNMNSDEKYITGKKILVNGADGIILGYGTRSSKEKPNMMISADMHEMDEYYIGGFTTSAGAEIFNTIAVPIEVNEKNKEYLKTLDSEIELPLTNVFGRTGIGTGKYSEVWEKADLRPKVDINRCRVCDSCEAQKMCPTNAIVRLDHLGKRTLPNENCFGCGVCVDSCTYGVYQMDRKSIMGIPITCRQSDRVRALKLAKELKKRIEKEEFKI

SEQ ID NO: 97 - protein, 389 aa, Methanotorris formicicus
MDKIVVTIDELKKMIRNNEEDKIDEIDIVTAATCGIMSGTMAIFHIPMKDKFRKAEKVYLNGIKSFPGPCPNEYLGSVDVVVFGTEHSEDYGGGFLFKDLVRGKEVEVKVECDGKIYTNEITLEEIPTARMIGTRMAFKNYVAVTNLGDEPIKTIFHRKFLRKGEASFSGCGEINPLQNMHVDEKELVGKKILLNGSEGVILGFGTRHSKEKPNIMISADMHNMDAYYLGGFITSAGSEVFNTIAIPIEVDEKHKEYLKKLDEYIDLPLTNVIGRNIVDVGKYSEVWKDVDLRPHVNVYRCRNCDFCIVEELCPTKAVRRVEHLGGRRLPNEDCFGCGVCATSCPYGIYQMKLGSILDIPITCRQSDRERALKLAEELKERIIKGEFDI

SEQ ID NO: 98 - protein, 398 aa, Methanothermococcus okinawensis
MDIGKDNNDKVVITANELKKMIRNNEEDKIDEIDIVTTATCGIMSGTMAVFHIPVADSGTFRKAEKIYLNGIEGYVGPCPNEYLGSVDAVVYGTNYTGEYGGGFLFKDLVSGKDIEIVVESEGKTYKKTLSLDDMPTAKMIGTRMAFKNYVAITNLSDESIKTIFHRRRLKKGEASFSGCGEINPLQNMKCDEKELFGKKVLLNGAEGIILGYGTRHSQNKPNIMISADMHNMDAYYLGGFITSAGVEVFNTIAIPVEVNEKNKEYLKTLDEDIELPLTNIIGRAPVGTGRYSEVWKDAELRPNVNIYRCRNCDTCIAEQLCPTKAIKRIPHLGGRRLPNENCFGCGVCVEACPYGIYQMKRNSILNIPLTCRQSDRERALKLAKDLKRRIEKGEFKL

SEQ ID NO: 99 - protein, 410 aa, Methanococcus aeolicus
MGSSDKPTKSNSEKVVITVDKLKQMIRNNEEDKIDEIDIITTATCGIMSGTMGIFHIPVAEPKSFRKAEKVYLNGVEGYVGPCPNEFLGTVDITVYGTNYTGNYGGGFLFKDLISKKEVAVEAISNGKTYNKTITIDDIPTAKMIGTRTAFKNYVALTNTGEKEESIKTIFHRRRLKKGEASFSGCGEINPLQNMKLSSNIKEAEKELLGKKILYNGAEGIILGYGTRHSAERPNIMISADIKEMDAHFVGGFVTSGGVEIYTTIAIPIEVNKENKEYLKTLDDDIPLTLTDVLGRIPVGMGTYSEVWKDCELRPKVNVERCRMCDVCIAQRLCPTNAIKPIKHLGNRRLPNEDCFGCGVCVEGCPYGIFSMKRNSICGTPITCRQSDRDRALKLARDLKRRIEKGEFEL

SEQ ID NO: 100 - protein, 380 aa, Methanopyrus kandleri
MPTVVRIDEFWDMTEGERRSVDAVTCATVGLMSGVYGILSFHVAEPGEVRRFEEVYLEGIRLPVGPCPNERLGLVECVVPATAHGDVSGAYLLRTVAEGNEFDVIAVADDGREYEATIGPEELERAMIASTRACFRNYSAIFNDGERPASTIFAPRPLEPGEASFSGCGGYNPLEHDPKLRLHRPGRRVLFCGAPGVITGKGTRSTRERPNLTLHANLTECDPTFMGEYRTSAGPENIAGVASVIAVTEETLPFLERDHSDAVLPVVKLSDRTPIAETTYADVWVQETVRWNPDRCENCDPCRLEEKCPYPYGDLRTGDCFHCGYCHEFCPAVEVDLGELEVEGRKLPIVARESSAYLAKKLMERAIEAVEEGKIDILRP